(19) United States
(12) Patent Application Publication
(10) Pub. No.: US 2023/0061319 A1
(43) Pub. Date: Mar. 2, 2023

(54) METHODS OF TREATING TUMORS

(71) Applicant: CoImmune, Inc., Durham, NC (US)

(72) Inventors: Joseph HORVATINOVICH, Durham, NC (US); Mark DEBENEDETTE, Durham, NC (US); Charles NICOLETTE, Durham, NC (US); Irina TCHEREPANOVA, Durham, NC (US)

(21) Appl. No.: 17/758,558

(22) PCT Filed: Jan. 8, 2021
(86) PCT No.: PCT/US2021/012753

§ 371 Date: Jul. 8, 2022

Related U.S. Application Data
(60) Provisional application No. 62/959,317, filed on Jan. 10, 2020.

(51) Int. Cl. A61K 39/00; A61K 31/436; A61K 39/395; A61K 31/167; A61K 31/573; A61K 31/664; A61K 31/519; A61K 31/5377; A61K 31/52; A61K 31/517; A61K 31/58; A61K 31/55; A61K 31/19; A61K 31/515; A61K 31/4166; A61K 31/454; A61K 31/4706; A61K 31/439; A61K 31/505; A61K 31/65; A61K 31/7056; A61P 35/00

(52) U.S. Cl. CPC A61K 39/001191; A61K 31/19; A61K 31/52; A61K 31/55; A61K 31/58; A61K 31/65; A61K 31/167; A61K 31/436; A61K 31/439; A61K 31/454; A61K 31/505; A61K 31/515; A61K 31/517; A61K 31/519; A61K 31/573; A61K 31/664; A61K 31/4166; A61K 31/4706; A61K 31/5377; A61K 31/7056; A61K 39/3955; A61P 35/00; A61K 2039/53; A61K 2039/545

(57) ABSTRACT

The present disclosure provides methods of treating a tumor (e.g., renal cell cancer) by administering an immunotherapy comprising dendritic cells loaded with RNA encoding a tumor antigen and a pharmaceutical which can decrease circulating IgG levels, block IgG-mediated activation of CD16⁺ T cells, decrease the concentration and/or function of B cells, reduce the frequency of CD38⁺ TGF-β⁺ B cells, decrease B cell secretion of TGF-β, and/or sustain the frequency of CD25⁺CD28⁺ CD4 and/or CD8 T cells.

### CROSS REFERENCE TO RELATED APPLICATIONS

This application claims priority to U.S. Provisional Application No. 62/959,317, filed Jan. 10, 2020, which is herein incorporated by reference in its entirety.

### FIELD OF THE DISCLOSURE

The present disclosure provides methods of treating a tumor (e.g., renal cell cancer) by administering an immunotherapy comprising dendritic cells loaded with RNA encoding a tumor antigen to a patient having a tumor and a pharmaceutical which can decrease circulating IgG levels, block IgG-mediated activation of CD16⁺ T cells, decrease the concentration and/or function of B cells (e.g., an mTOR inhibitor), reduce the frequency of CD38⁺ TGF-β⁺ B cells, decrease B cell secretion of TGF-β, and/or sustain the frequency of CD25⁺CD28⁺ CD4 and/or CD8 T cells.

### BACKGROUND OF THE DISCLOSURE

Kidney cancer is among the 10 most common cancers in both men and women. Overall, the lifetime risk for developing kidney cancer in men is about 1 in 48 and for women about 1 in 83. Siegel RL et al. Cancer Journal for Clinicians 2019;69(1):7-34 (2019). According to the American Cancer Society’s estimates for kidney cancer in the United States for 2019, approximately 73,820 new cases of kidney cancer (44,120 in men and 29,700 in women) will occur and approximately 14,770 people (9,820 men and 4,950 women) will die from this disease. These numbers include all types of kidney and renal pelvis cancers. According to the National Comprehensive Cancer Network (“NCCN”) and the National Cancer Database (“NCDB”), it is estimated that 90% of new kidney cancer cases each year are renal cell carcinoma (“RCC”).

The initial treatment for most patients with RCC, including metastatic RCC (“mRCC”), is surgical removal of the tumor, usually requiring partial or complete removal of the affected kidney, referred to as nephrectomy. In the absence of metastatic disease, the NCCN recommends observation after nephrectomy, although systemic therapies are recommended for patients who are believed to be at higher risk of relapse. Notably, patients whose tumors have metastasized to other organs beyond the primary kidney at the time of diagnosis are considered to have newly diagnosed mRCC and have the poorest overall prognosis and survival. For patients who present with mRCC upon diagnosis or as a result of a relapse from an earlier stage of RCC, the NCCN recommends systemic treatment with currently available therapies, except in the rare instances where metastatic lesions can be removed by surgery alone.

mRCC is generally resistant to conventional systemic approaches such as chemotherapy, radiation and hormonal therapies. Although mRCC has been treated with cytokine-based immunotherapies such as interferon-α and IL-2, which have demonstrated a clinical benefit in a small number of mRCC patients, these therapies have been shown to have severe toxicities which limit their use, including cardiopulmonary, neuropsychiatric, dermatologic, renal, hepatic, and hematologic side effects. Although high-dose IL-2 has demonstrated complete mRCC remissions, its toxicity restricts its use to a small minority of patients.

In the past decade, several agents, such as Sutent® (sunitinib), Votrient® (pazopanib), Torisel® (temsirolimus), Nexavar® (sorafenib), Avastin® (bevacizumab) plus interferon-α, Afinitor® (everolimus), Inlyta® (axitinib) and most recently cabozantanib, nivolamab and ipilimimab, and nivolimab and a tyrosine kinase inhibitor (“TKI”) have been approved for the treatment of mRCC.

Although most of these new agents have demonstrated prolonged progression-free survival as compared to interferon-α, they are rarely associated with durable remissions or survival, particularly in patients who are not classified as favorable risk at the time of treatment. In addition, each of these new agents has shortcomings that limit its use in the treatment of mRCC, including significant toxicities, such as neutropenia and other hematologic toxicities, fatigue, diarrhea, hand-foot syndrome, hypertension, and other cardiovascular effects.

Accordingly, there is a need to develop a safer and more effective treatment for metastatic RCC.

### BRIEF SUMMARY OF THE DISCLOSURE

One aspect of the present disclosure is directed to methods of treating a tumor, comprising the sequential steps of: (a) administering a dose regimen of an immunotherapy comprising dendritic cells loaded with RNA encoding a tumor antigen to a patient having a tumor; and (b) after administration of at least one dose of the immunotherapy of step (a), administering a dose regimen of a pharmaceutical which can cause one or more of the following: (i) decrease circulating IgG levels, (ii) block IgG-mediated activation of CD16⁺ T cells, (iii) decrease the concentration and/or function of B cells, (iv) reduce the frequency of CD38⁺ TGF-β⁺ B cells, (v) decrease B cell secretion of TGF-β, and (vi) sustain the frequency of CD25⁺CD28⁺ CD4 and/or CD8 T cells.

In certain aspects, administration of the pharmaceutical occurs after tumor progression.

In some aspects, the pharmaceutical is an mTOR inhibitor. In some aspects, the mTOR inhibitor is rapamycin or a rapamycin analog. In certain aspects, the rapamycin analog is selected from the group consisting of everolimus, temsirolimus, sirolimus, and ridaforolimus. In some aspects, the rapamycin analog is everolimus. In certain aspects, everolimus is administered once per day. In some aspects, about 2.5 mg to about 20 mg of everolimus is administered once per day. In some aspects, about 10 mg of everolimus is administered once per day.

In some aspects, the rapamycin analog is temsirolimus. In certain aspects, temsirolimus is administered once per week. In some aspects, about 25 mg of temsirolimus is administered once per week. In certain aspects, a first dose of the mTOR inhibitor is administered after progression of the tumor.

In some aspects, the pharmaceutical decreases the function of B cells and is selected from the group consisting of: natalizumab, teriflunomide, and ofatumumab.

In some aspects, the pharmaceutical decreases the concentration of B cells and is selected from the group consisting of: prednisone, cyclophosphamide, methotrexate, mycophenolate mofetil, azathioprine, trimetrexate, cortisol, prednisolone, methylprednisolone, dexamethasone, metamethasone, triamcinolone, denosumab, triamcinolone acetonide, atacicept, ocrelizumab, obinutuzumab, bevacizumab, and inotuzumab ozogamicin.

In some aspects, the pharmaceutical decreases circulating levels of IgG and is selected from the group consisting of carbamazepine, sodium valproate, phenobarbital, phenytoin, lenalidomide, cloroquine, quinine, amodiaquine, pyrimethamine, proguanil, sulfonamides, mefloquine, atovaquone, primaquine, artemisinin, halofantrine, doxycycline, clindamycin, captopril, cortisol, prednisone, prednisolone, methylprednysolone, dexamethasone, metamethasone, trimcinolone, fludrocortisone acetate, deoxycorticostetone acetate, fenclofenac, gold salts, penicillamine, and sulfasalazine.

In other aspects, the pharmaceutical blocks IgG-mediated activation of CD16⁺ T-regulatory (Treg) cells.

In some aspects, the pharmaceutical is an anti-CD16 antibody or an antibody that cross-competes with the anti-CD16 antibody for binding to the same epitope or an antibody that binds to the same epitope as the anti-CD16 antibody.

In some aspects, the pharmaceutical is the 3G8 antibody, the B73.1 antibody or the CB16 antibody, or an antibody that cross-competes with the 3G8 antibody, the B73.1 antibody, or the CB16 antibody for binding to the same epitope or an antibody that binds to the same epitope as the 3G8 antibody, the B73.1 antibody, or the CB16 antibody.

In some aspects, the immunotherapy is CMN-001. In certain aspects, CMN-001 is administered about once every three weeks.

In some aspects, the regimen of immunotherapy continues after the initiation of the dose regimen of the pharmaceutical.

In some aspects, the tumor is renal cell cancer. In certain aspects, the tumor is metastatic renal cell cancer. In some aspects, the tumor is the clear cell type.

In certain aspects, the tumor is selected from the group consisting of: breast cancer, pancreatic cancer, astrocytoma, glioblastoma multiforme, melanoma, lymphoma, and Waldenstrom macroglobulinaemia.

In some aspects, the patient is a poor risk or an intermediate risk human patient. In certain aspects, the poor risk patient exhibits three or more of the following risk factors: (i) time from diagnosis to the initiation of systemic therapeutic treatment of less than one year, (ii) low levels of hemoglobin, (iii) elevated corrected calcium levels, (iv) diminished patient performance status or physical functioning, (v) elevated levels of neutrophils, and (vi) elevated platelet count.

In some aspects, the tumor antigen is autologous to the patient.

Also provided herein are methods of decreasing circulating IgG levels, blocking IgG-mediated activation of CD16⁺ T cells, and/or decreasing the concentration or function of B cells in a patient having a tumor, comprising the sequential steps of: (a) administering a dose regimen of an immunotherapy comprising dendritic cells loaded with RNA encoding a tumor antigen to the patient; and (b) after administration of at least one dose of the immunotherapy of step (a), administering a dose regimen of a pharmaceutical which can cause one or more of the following: (i) decrease circulating IgG levels, (ii) block IgG-mediated activation of CD16⁺ T cells, (iii) decrease the concentration or function of B cells, (iv) reduce the frequency of CD38⁺ TGF-β⁺ B cells, (v) decrease B cell secretion of TGFβ, and (vi) sustain the frequency of CD25⁺CD28⁺ CD4 and/or CD8 T cells.

Also provided herein are methods of modulating Programmed Cell Death Protein 1 (PD1) expression on CD8+ T cells in a patient having a tumor, comprising the sequential steps of: (a) administering a dose regimen of an immunotherapy comprising dendritic cells loaded with RNA encoding a tumor antigen to the patient; and (b) after administration of at least one dose of the immunotherapy of step (a), administering a dose regimen of a pharmaceutical which can cause one or more of the following: (i) decrease circulating IgG levels, (ii) block IgG-mediated activation of CD16+ T cells, (iii) decrease the concentration and/or function of B cells, (iv) reduce the frequency of CD38⁺ TGF-β⁺ B cells, (v) decrease B cell secretion of TGF-β, and (vi) sustain the frequency of CD25⁺CD28⁺ CD4 and/or CD8 T cells.

Other features and advantages of the present disclosure will be apparent from the following detailed description and examples which should not be construed as limiting. The contents of all cited references, including scientific articles, newspaper reports, GenBank entries, patents and patent applications cited throughout this application are expressly incorporated herein by reference.

### BRIEF DESCRIPTION OF THE DRAWINGS

FIGS. 1A-1D show induction of CMV pp65 specific memory recall cytotoxic T lymphocytes (CTLs) in the presence of two mTOR inhibitors, everolimus and temsirolimus. The percentage of CMV pp65 specific CTLs were measured in the absence of the mTOR inhibitors, everolimus and temsirolimus (“untreated”) and in the presence of 10 ng/ml (“10 ng”), 100 ng/ml (“100 ng”), or 1 ug/ml (“1 ug”) of everolimus (FIG. 1A) or temsirolimus (FIG. 1C). The absolute numbers of CMV pp65 specific CTLs were measured in the absence of the mTOR inhibitors (“untreated”) and in the presence of 10 ng/ml (“10 ng”), 100 ng/ml (“100 ng”), or 1 ug/ml (“1 ug”) of everolimus (FIG. 1B) or temsirolimus (FIG. 1B).

FIGS. 2A-2B show induction of CMV pp65 specific recall CTL effector function in the presence of two mTOR inhibitors, everolimus and temsirolimus. The absolute numbers of CMV pp56 specific CTLs that secrete IFN-y, TNF-α or IL-2, or express CD107a in response to CMV pp65 peptide pulsed target cells were measured in the absence of the mTOR inhibitors, everolimus and temsirolimus (“untreated”) and in the presence of 10 ng/ml (“10 ng”), 100 ng/ml (“100 ng”), or 1 ug/ml (“1 ug”) of everolimus (FIG. 2A) or temsirolimus (FIG. 2B).

FIGS. 3A-3D show induction of MART-1 specific memory recall CTLs in the presence of two mTOR inhibitors, everolimus and temsirolimus. The percentage of MART-1 specific CTLs were measured in the absence of the mTOR inhibitors, everolimus and temsirolimus ("untreated") and in the presence of 10 ng/ml (“10 ng”), 100 ng/ml (“100 ng ”), and 1 ug/ml (“1 ug”) of everolimus (FIG. 3A) or temsirolimus (FIG. 3C). The absolute numbers of MART-1 specific CTLs were measured in the absence of the mTOR inhibitors (“untreated”) and in the presence of 10 ng/ml (“10 ng”), 100 ng/ml (“100 ng ”), and 1 ug/ml (“1 ug”) of everolimus (FIG. 3B) or temsirolimus (FIG. 3B).

FIGS. 4A-4B show induction of MART-1 specific recall CTL effector function in the presence of two mTOR inhibitors, everolimus and temsirolimus. The absolute numbers of MART-1 specific CTLs that secrete IFN-y, TNF-α or IL-2, or express CD107a in response to MART-1 peptide pulsed target cells were measured in the absence of the mTOR inhibitors, everolimus and temsirolimus ("untreated") and in the presence of 10 ng/ml (“10 ng”), 100 ng/ml (“100 ng”), or 1 ug/ml (“1 ug”) of everolimus (FIG. 4A) or temsirolimus (FIG. 4B).

FIGS. 5A-5D show increased activated NK cells and memory T cells with decreased IgG binding regulatory cells. Data generated from CTL cultures stimulated with CMV antigen encoding DCs was plotted to show the relationship between activated NK cells (cells/ml) and activated CD4⁺ T cells (cells/ml) (FIG. 5A), activated NK cells (cells/ml) and IgG binding regulatory CD4⁺T cells (FIG. 5B), CMV+ (specific) CTLs (cells/ml) and activated CD4⁺ T cells (cells/ml) (FIG. 5C), and CMV+ CTLs (cells/ml) and IgG binding regulatory CD4⁺ T cells (FIG. 5D).

FIGS. 6A-6F show NK cell and T cell proliferation in mRCC patients measured by the percentage (%) of (Ki67⁺) programmed cell death protein 1 (“PD1”)⁻ cells. FIG. 6A shows the percentage (%) of Ki67⁺PD1⁻ NK cells (CD3-CD16+CD56⁺) from B cell depleted PBMCs (“B cell depleted”) or PBMCs without B cell depletion (“none”) in culture without DCs stimulation at days 1-8. FIG. 6B shows the percentage (%) of Ki67⁺PD1⁻ NK cells (CD3⁻CD16⁺CD56⁺) from B cell depleted PBMCs (“B cell depleted”) or PBMCs without B cell depletion (“none”) in culture with DCs stimulation at days 1-8. FIG. 6C shows the percentage (%) of Ki67⁺PD1⁻ CD4⁺ T cells (CD3⁺CD4⁺) from B cell depleted PBMCs (“B cell depleted”) or PBMCs without B cell depletion (“none”) in culture without DCs stimulation at days 1-8. FIG. 6D shows the percentage (%) of Ki67⁺PD1⁻ CD4⁺ T cells (CD3⁺CD4⁺) from B cell depleted PBMCs (“B cell depleted”) or PBMCs without B cell depletion (“none”) in culture with DCs stimulation at days 1-8. FIG. 6E shows the percentage (%) of Ki67⁺PD1⁻ CD8⁺T cells (CD3⁺CD8⁺) from B cell depleted PBMCs (“B cell depleted”) or PBMCs without B cell depletion (“none”) in culture without DCs stimulation at days 1-8. FIG. 6F shows the percentage (%) of Ki67⁺PD1⁻ CD8⁺ T cells (CD3⁺CD8⁺) from B cell depleted PBMCs (“B cell depleted”) or PBMCs without B cell depletion (“none”) in culture with DCs stimulation at days 1-8.

FIGS. 7A-7F show in vitro stimulation of PBMCs from an mRCC patient. The percentage of CD3⁺CD4^{low}, FoxP3⁺ cells (Q1 gate), CD3⁺CD4^{hi,} FoxP3⁺ cells (Q2 gate), and of CD3⁺CD4^{low}, FoxP3⁻ cells (Q4 gate) was measured using flow cytometry on day 7 after cultured PBMCs from an mRCC patient were stimulated with PME-CD40L DCs (FIG. 7A) or without PME-CD40L DCs stimulation (FIG. 7D). The expression of CD25 (FIG. 7B), intracellular IgG (FIG. 7C), PD1 (FIG. 7E), and chemokine receptor CXCR4 (FIG. 7F) was measured in CD3⁺CD4^{low}, FoxP3⁺ cells (dashed line in histogram), CD3⁺CD4^{hi}, FoxP3⁺ cells (solid line in histogram), and of CD3⁺CD4^{low}, FoxP3⁻ cells (shaded histogram).

FIGS. 8A-8G show IgG-immune complex binding and internalization by CD4⁺ T cells during 8 days of PME-CD40L DCs stimulation as measured using flow cytometry. IgG detection in CD4^{hi} expressing T cells was measured in B cell depleted PBMCs from an mRCC patient (FIG. 8C) or when the anti-CD16 antibody clones 3G8 (FIG. 8D), B73.1 (FIG. 8E) or CB16 (FIG. 8F), or an isotype control antibody MPOC-21 (FIG. 8B) was added to the PBMC cultures from an mRCC patient, relative to cells without an anti-CD16 antibody (“no antibody”) (FIG. 8A). FIG. 8G shows the percentage of CD3⁺CD4^{hi} cells binding IgG in the absence of an anti-CD16 antibody (“none”), in the presence of MPOC-21, 3G8, B73.1 or CB16, or with B cell depletion of PBMCs from an mRCC patient.

FIGS. 9A-9G show PD1 expression in CD8⁺ T cells after 8 days of PME-CD40L DCs stimulation. PD1 expression in proliferating (Ki67⁺) CD8⁺ T cells was measured in B cell depleted PBMCs from an mRCC patient (FIG. (C) or when the anti-CD16 antibody clones 3G8 (FIG. 9D), B73.1 (FIG. 9E) or CB16 (FIG. 9F), or an isotype control antibody MPOC-21 (FIG. 9B) was added to the PBMC cultures from an mRCC patient, relative to the cells without an anti-CD16 antibody (“no antibody”) (FIG. 9A). FIG. 9G shows the percentage of PD1 negative proliferating (Ki67⁺) CD8⁺ T cells in the absence of an anti-CD16 antibody (“none”), in the presence of MPOC-21, 3G8, B73.1 or CB16, or with B cell depletion of PBMCs from an mRCC patient.

FIGS. 10A-10Y show the effect of inhibition of IgG-immune complex binding and internalization by CD4⁺ T cells on antigen specific CTL expansion. The expression of Foxp3 in CD4⁺CD25⁺ cells was measured in healthy donor PBMCs in the absence of the DCs (electroporated with pp65 mRNA) (“None”) (FIG. 10A) or stimulated with DCs electroporated with RNA encoding pp65 CMV protein only (DC^{CMV}) in the presence of the anti-CD16 antibody (“3G8”) (FIG. 10B), the DCs (electroporated with pp65 mRNA) (“DC”) (FIG. 10C), or the DCs (electroporated with pp65 mRNA) and the anti-CD16 antibody (“DC+3G8”) (FIG. 10D). The expression of IgG in CD4⁺CD25⁺Foxp3⁺ cells was measured in healthy donor PBMCs in the absence of the DCs (electroporated with pp65 mRNA) (“None”) (FIG. 10E) or stimulated with DCs electroporated with RNA encoding pp65 CMV protein only (DC^{CMV}) in the presence of the anti-CD16 antibody (“3G8”) (FIG. 10F), the DCs (electroporated with pp65 mRNA) (“DC”) (FIG. 10G), or the DCs (electroporated with pp65 mRNA) and the anti-CD16 antibody (“DC+3G8”) (FIG. 10H). The PD1 expression in CD3⁺CD8⁺ cells was measured in healthy donor PBMCs in the absence of the DCs (electroporated with pp65 mRNA) ("None") (FIG. 10I) or stimulated with DCs electroporated with RNA encoding pp65 CMV protein only (DC^{CMV}) in the presence of the anti-CD16 antibody ("3G8") (FIG. 10J), the DCs (electroporated with pp65 mRNA) ("DC") (FIG. 10K), or the DCs (electroporated with pp65 mRNA) and the anti-CD16 antibody ("DC+3G8") (FIG. 10L).The expression of Foxp3 in CD4⁺CD25⁺ cells was measured in healthy donor PBMCs in the absence of the DCs (electroporated with pp65 mRNA) ("None") (FIG. 10M) or stimulated with PME-CD40L DCs electroporated with RNA encoding pp65 CMV protein (DC^{CD40L+CMV}) in the presence of the anti-CD16 antibody ("3G8") (FIG. 10N), the DCs (electroporated with pp65 mRNA) ("DC") (FIG. 10O), or the DCs (electroporated with pp65 mRNA) and the anti-CD16 antibody ("DC+3G8") (FIG. 10P). The expression of IgG in CD4⁺CD25⁺Foxp3⁺ cells was measured in healthy donor PBMCs in the absence of the DCs (electroporated with pp65 mRNA) (“None”) (FIG. 10Q) or stimulated with PME-CD40L DCs electroporated with RNA encoding pp65 CMV protein (DC^{CD40L+CMV}) in the presence of the anti-CD16 antibody (“3G8”) (FIG. 10R), the DCs (electroporated with pp65 mRNA) (“DC”) (FIG. 10S), or the DCs (electroporated with pp65 mRNA) and the anti-CD16 antibody (“DC+3G8”) (FIG. 10T). The PD1 expression in CD3⁺CD8⁺ cells was measured in healthy donor PBMCs in the absence of the DCs (electroporated with pp65 mRNA) (“None”) (FIG. 10U) or stimulated with PME-CD40L DCs electroporated with RNA encoding pp65 CMV protein (DC^{CD40L+CMV}) in the presence of the anti-CD16 antibody (“3G8”) (FIG. 10V), the DCs (electroporated with pp65 mRNA) (“DC”) (FIG. 10W), or the DCs (electroporated with pp65 mRNA) and the anti-CD16 antibody (“DC+3G8”) (FIG. 10X). Correlation analysis for DC stimulated PBMCs shows a correlation for IgG detection in CD4⁺FoxP3⁺ (“IgG⁺,% of CD4⁺FoxP3⁺”) and PD1 expression on proliferating CD3⁺CD8⁺ T cells (“Ki67⁺PD1⁺, % of CD3⁺CD8⁺”).

FIGS. 11A-11L show the effect of inhibition of IgG-immune complex binding and internalization by CD4⁺ T cells on PD1 expression on antigen specific CD8⁺T cells. The expression of IgG in CD3⁺CD4⁺CD25⁺CD45RA⁻ cells was measured in healthy donor PBMCs stimulated with DC^{CD40L+CMV} (“DC:day0”) (FIG. 11A), DC^{CD40L+CMV} and the anti-CD16 antibody (“DC+3G8:day0”), (FIG. 11B), PBMCs stimulated with DC^{CD40L+CMV} and re-stimulated with DC^{CD40L+CMV} on day 6 (“DC:day0,6”) (FIG. 11C), or PBMCs stimulated with DC^{CD40L+CMV} and the anti-CD16 antibody and then re-stimulated after 6 days (“DC+3G8:day0,6”), (FIG. 11D). The expression of PD1 in CD3⁺CD8⁺cells was measured in healthy donor PBMCs stimulated with DC^{CD40L+CMV} (“DC:day0”) (FIG. 11E), DC^{CD40L+CMV} and the anti-CD16 antibody (“DC+3G8:day0”), (FIG. 11F), PBMCs stimulated with DC^{CD40L+CMV} and re-stimulated with DC^{CD40L+CMV} on day 6 (“DC:day0,6”) (FIG. 11G), or PBMCs stimulated with DC^{CD40L+CMV} and the anti-CD16 antibody and then re-stimulated after 6 days (“DC+3G8:day0,6”), (FIG. 11H).

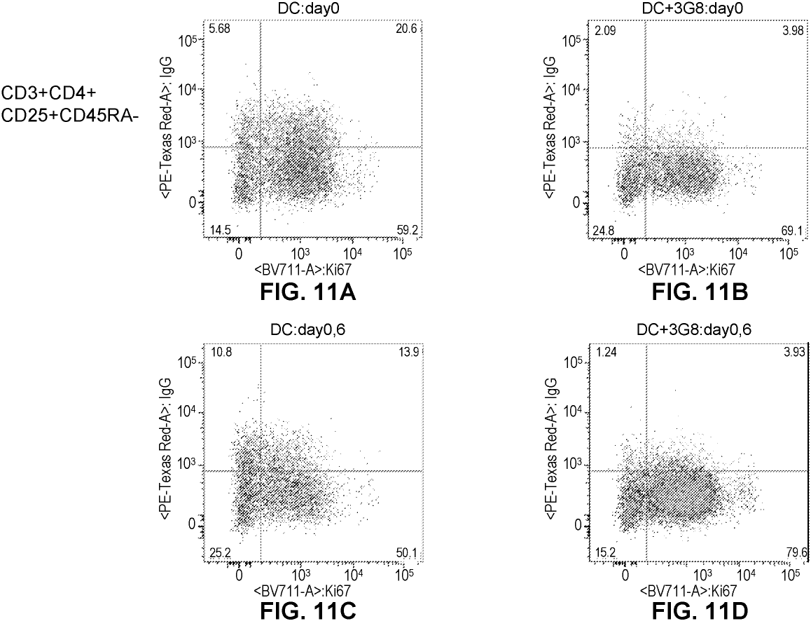

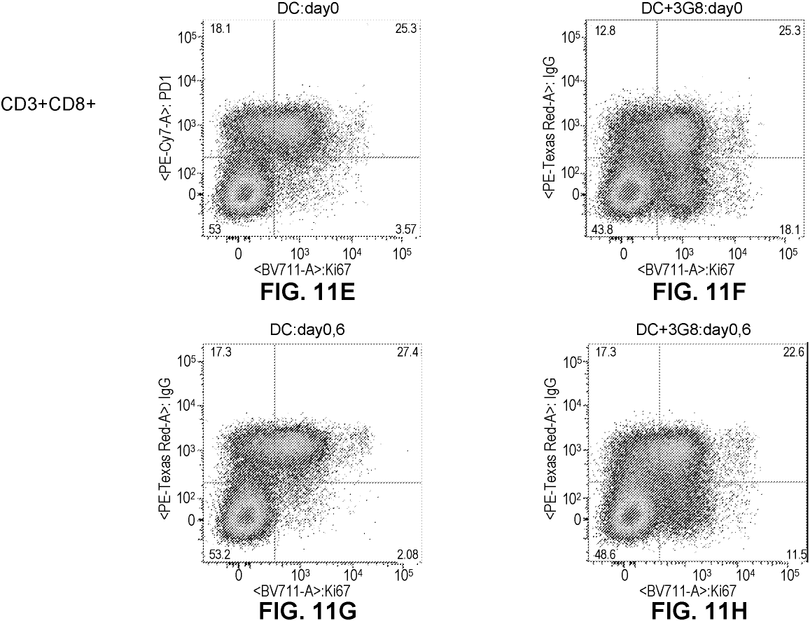

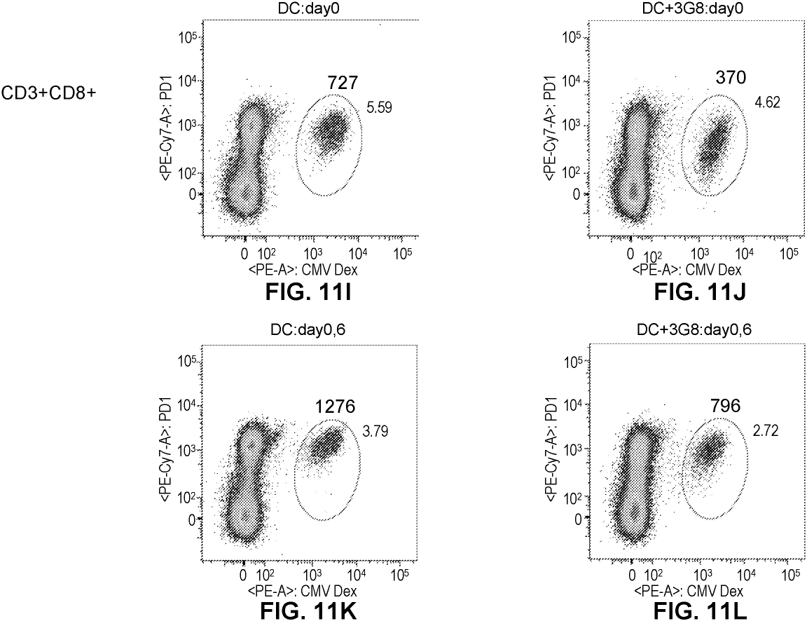

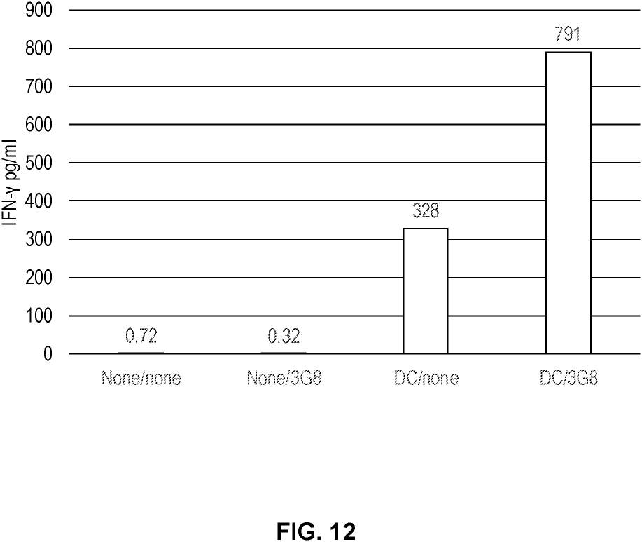

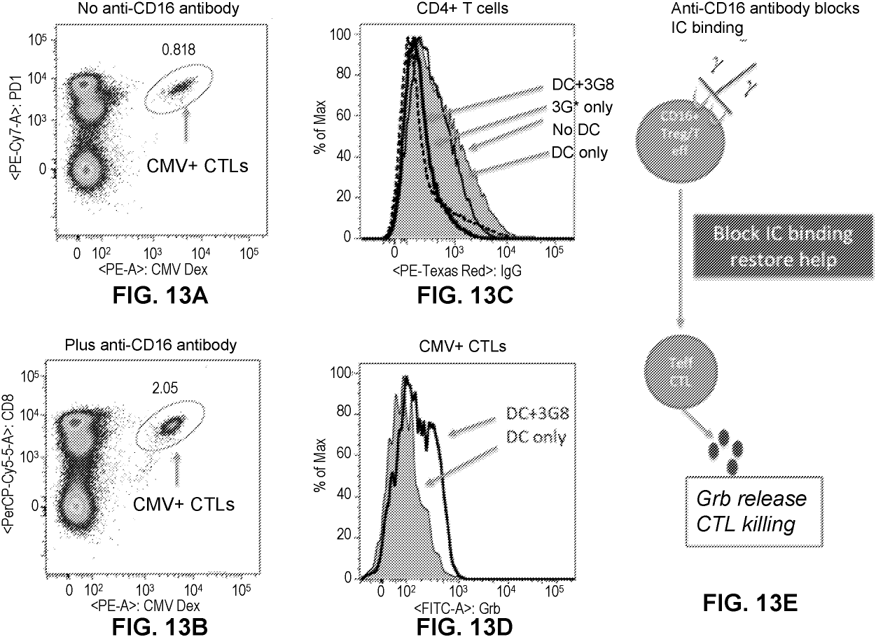

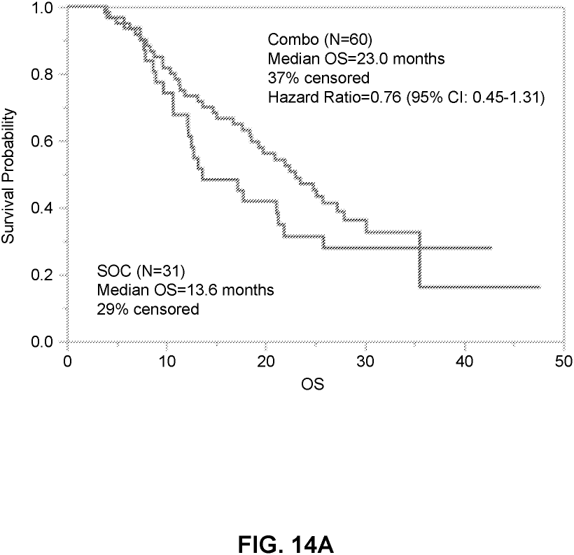

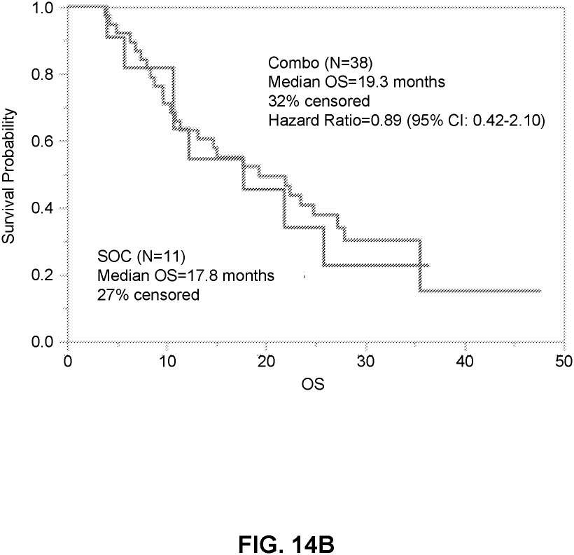

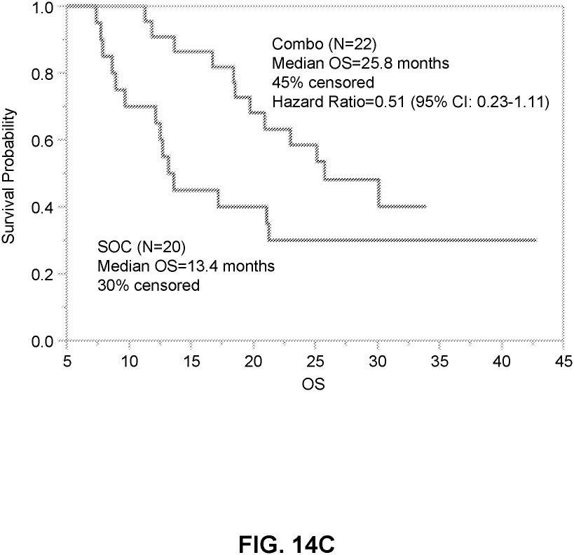

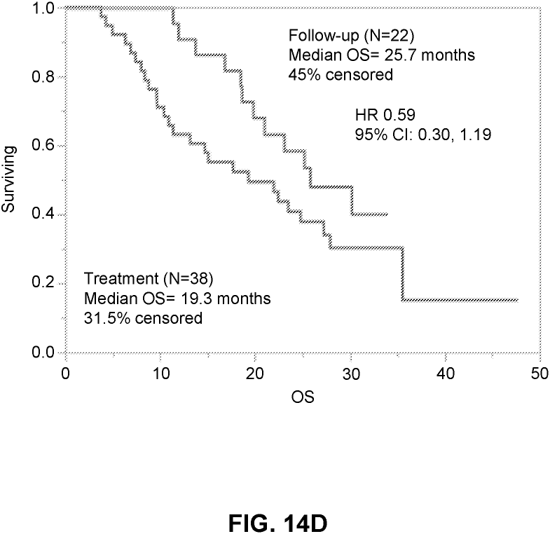

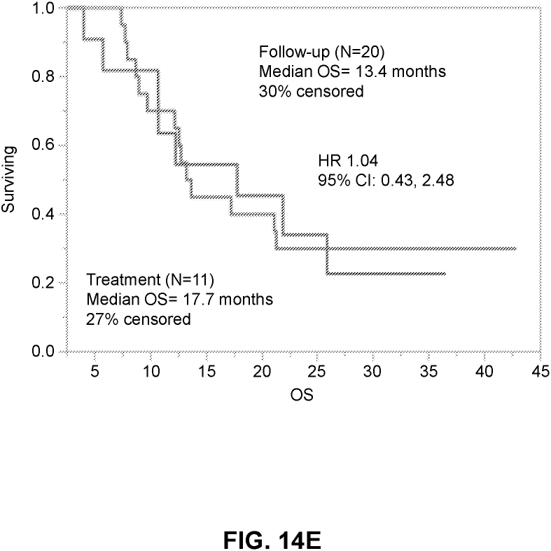

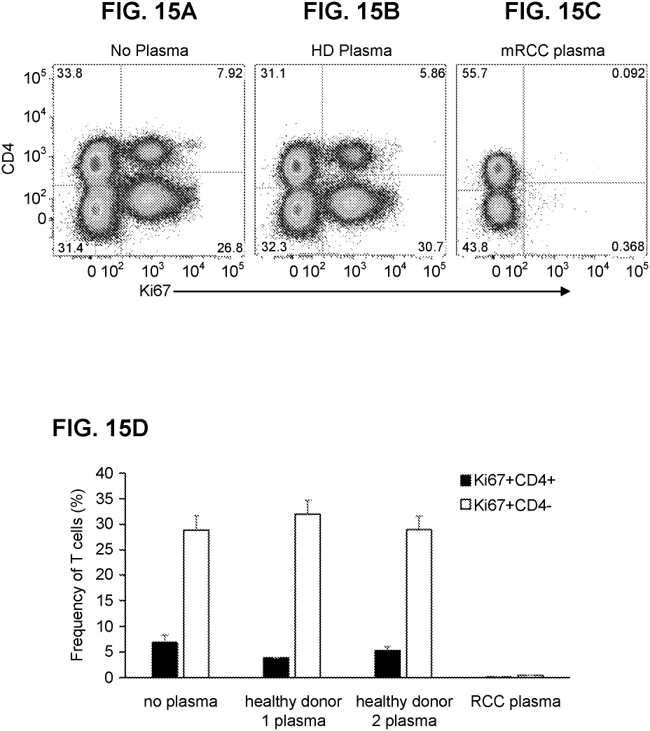

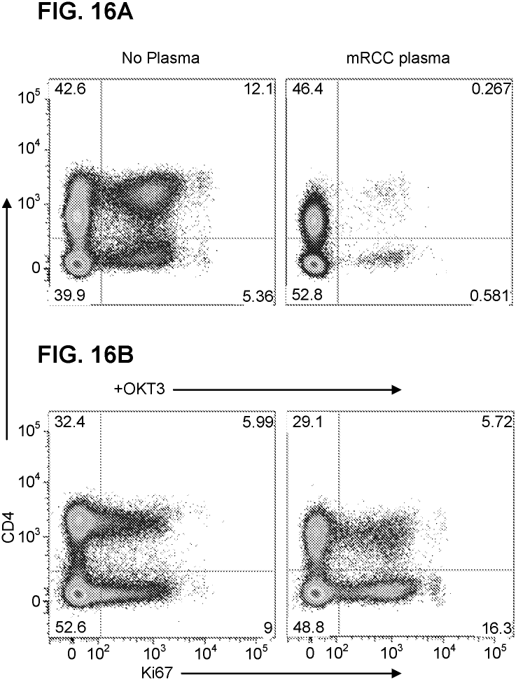

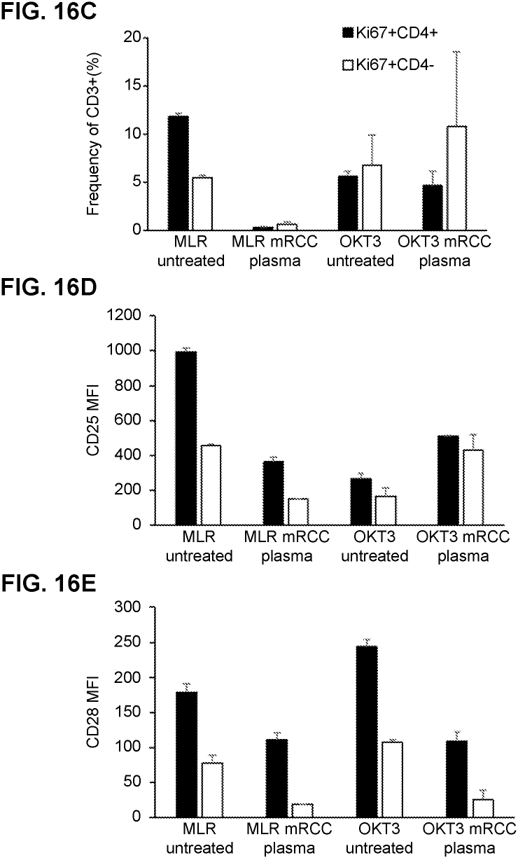

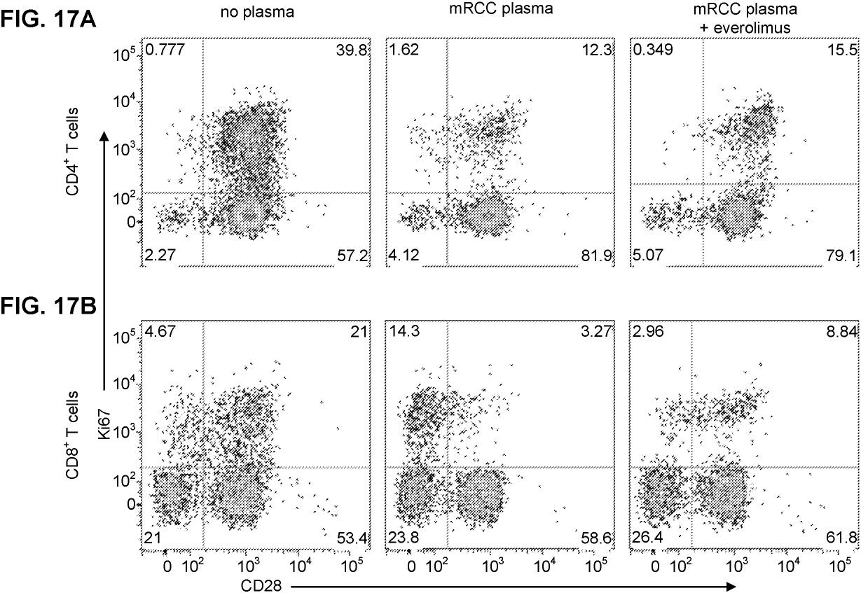

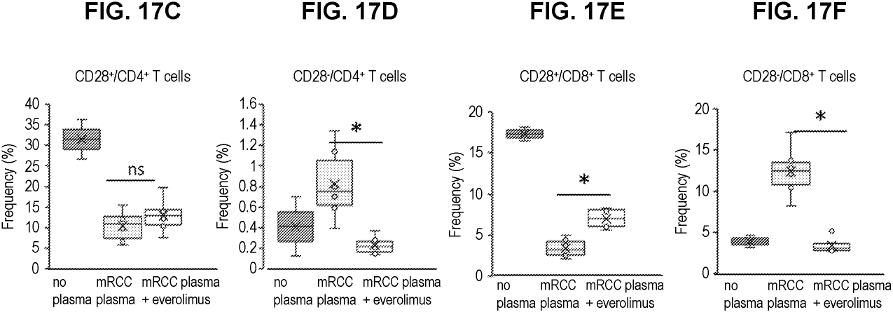

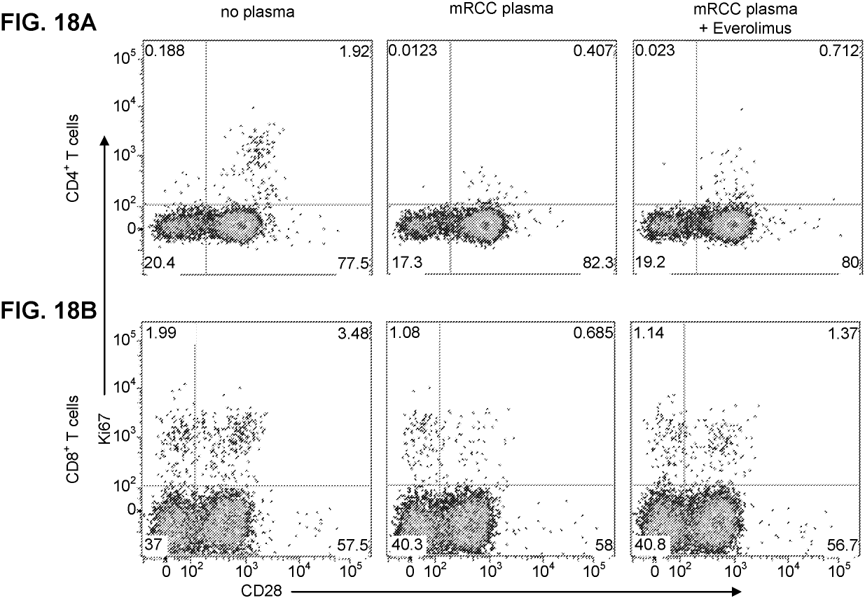

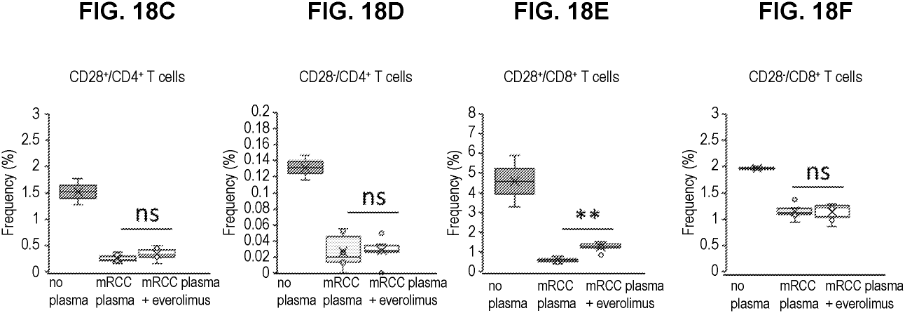

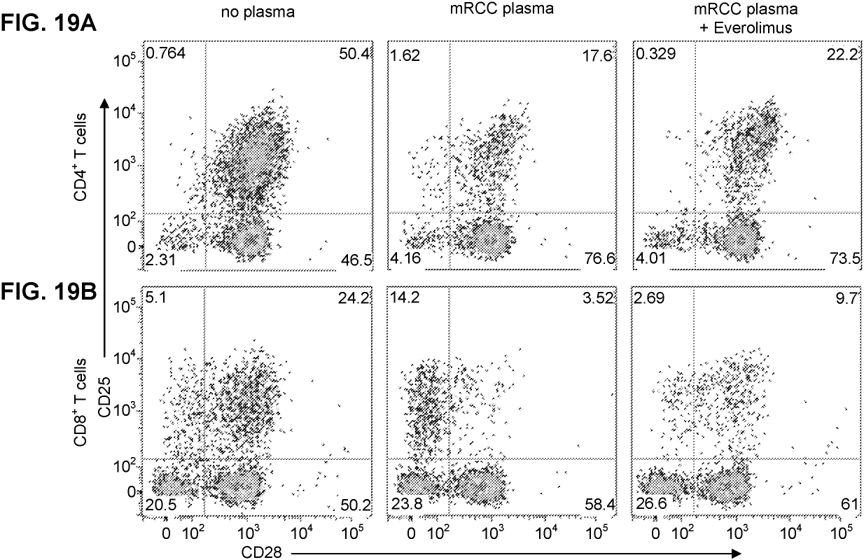

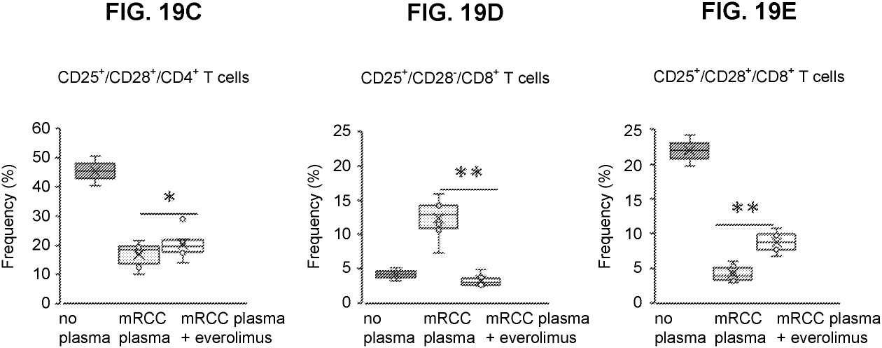

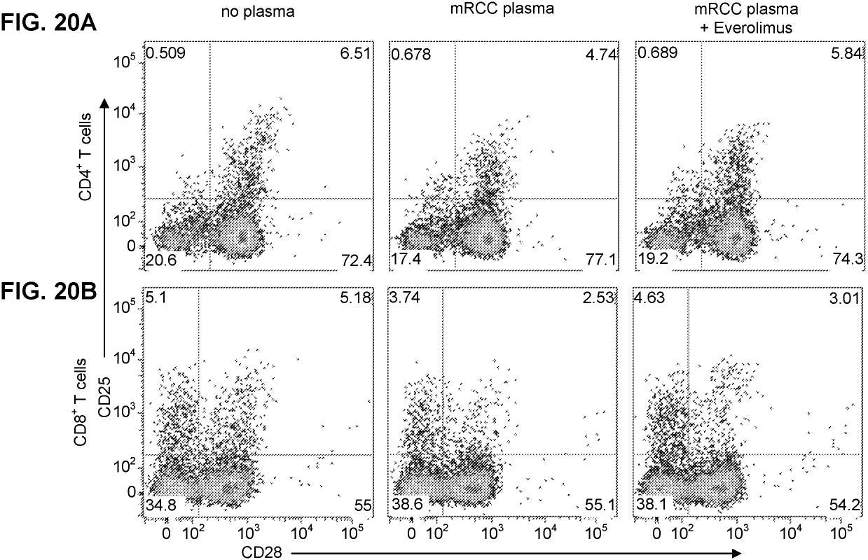

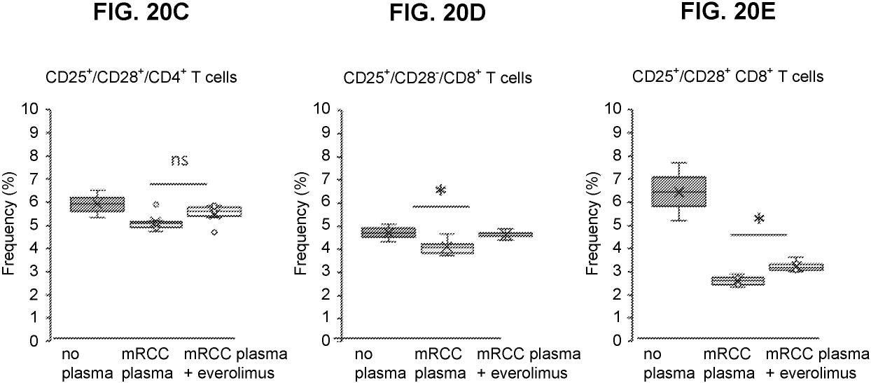

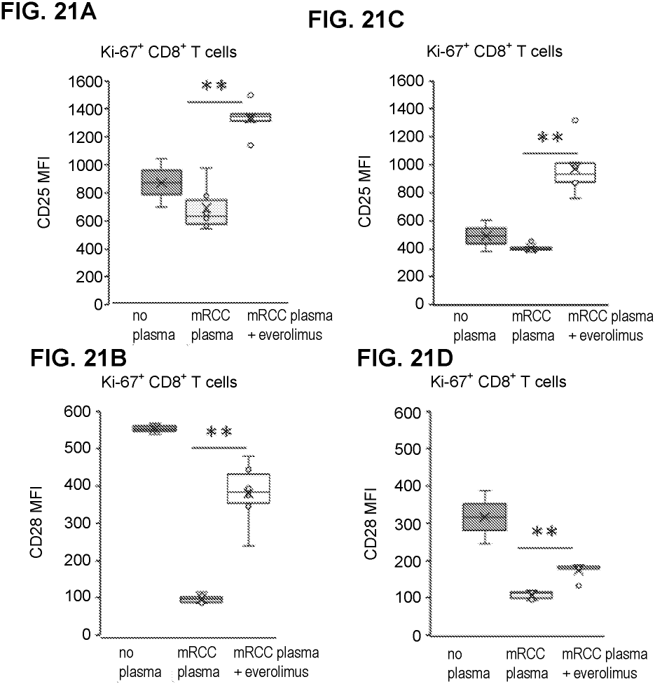

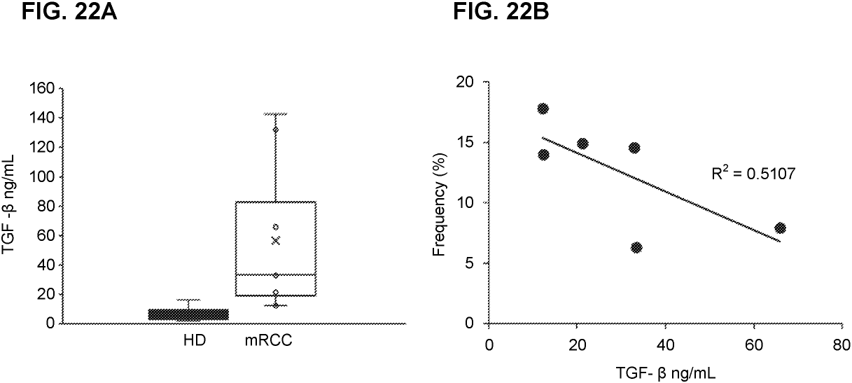

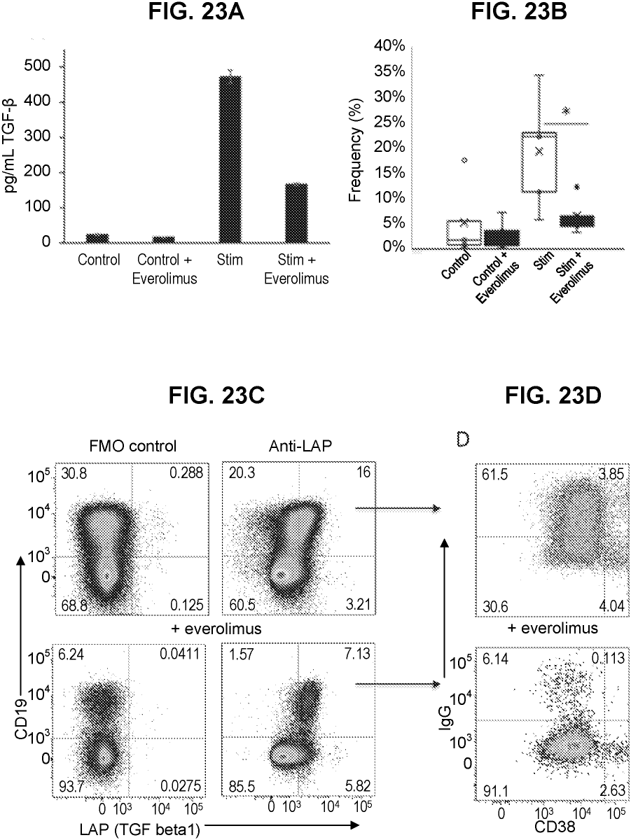

The expression of PD1 in CD3⁺CD8⁺cells was measured in healthy donor PBMCs stimulated with DC^{CD40L+CMV} (“DC:day0”) (FIG. 11E), DC^{CD40L+CMV} and the anti-CD16 antibody (“DC+3G8:day0”), (FIG. 11F), PBMCs stimulated with DC^{CD40L+CMV} and re-stimulated with DC^{CD40L+CMV} on day 6 (“DC:day0,6”) (FIG. 11G), or PBMCs stimulated with DC^{CD40L+CMV} and the anti-CD16 antibody and then re-stimulated after 6 days (“DC+3G8:day0,6”), (FIG. 11H). The expression of PD1 in CMV dextramer positive CD3⁺CD8⁺ cells was measured in healthy donor PBMCs stimulated with DC^{CD40L+CMV} (“DC:day0”) (FIG. 11I), DC^{CD40L+CMV}and the anti-CD16 antibody (“DC+3G8:day0”), (FIG. 11J), PBMCs stimulated with DC^{CD40L+CMV} and re-stimulated with DC^{CD40L+CMV} on day 6 (“DC:day0,6”) (FIG. 11K), or PBMCs stimulated with DC^{CD40L+CMV} and the anti-CD16 antibody and then re-stimulated after 6 days (“DC+3G8:day0,6”), (FIG. 11L).

FIG. 12 shows INF-γ secretion (pg/ml) by antigen specific memory T cells measured in a normal donor PBMCs in the absence of the DCs (electroporated with pp65 mRNA) and the anti-CD16 antibody (“None/None”), and in the presence of the anti-CD16 antibody (“None/3G8”), the PME-CD40L DCs electroporated with RNA encoding pp65 CMV protein (DC^{CD40L+CMV}) (“DC/None”), or the DC^{CD40L+CMV} and the anti-CD16 antibody (“DC/3G8”).

FIGS. 13A-13E show blocking of immunoglobulin complex (“IC”) binding by anti CD16 antibody. The percentage of CMV pp65 specific CTLs was measured in the absence (FIG. 13A) and the presence (FIG. 13B) of an anti-CD16 antibody using flow cytometry. The percentage of intracellular IgG in CD4⁺ T cells was measured in the presence of the DCs (electroporated with pp65 mRNA) and the anti-CD16 antibody (“DC+3G8”), the anti-CD16 antibody (“3G8 only”), or the DCs (electroporated with pp65 mRNA) (“DC only”), and in the absence of the DCs and the anti-CD16 antibody (“No DC”) (FIG. 13C) using flow cytometry. The percent of Granzyme B (“Grb”) production in CMV pp65 specific CTLs was measured in the presence of the DCs (electroporated with pp65 mRNA) and the anti-CD16 antibody (“DC+3G8”) or the DCs (electroporated with pp65 mRNA) (“DC only”) (FIG. 13D) using flow cytometry. The mechanism of anti-CD16 antibody blocking of IC binding is shown in FIG. 13E.

FIGS. 14A-14E show survival estimates in mRCC patients receiving everolimus in combination with a DC therapy. FIG. 14A shows Kaplan-Meier analysis of overall survival in the Phase 3 trial of Autologous Dendritic Cell Immunotherapy Plus Standard Treatment of Advanced Renal Cell Carcinoma (ADAPT) (including everolimus as a subsequent treatment). The everolimus treated population included subjects who received doses of CMN-001 (a DC therapy) (“Combo (N=60”)) or at least 1 or more doses of sunitinib (standard of care treatment (SOC) (“SOC (N=31)”). FIG. 14B shows Kaplan-Meier analysis of overall survival in the Phase 3 ADAPT trial (including everolimus as subsequent treatment in the treatment phase). The everolimus treated population included subjects who received everolimus during the treatment phase with the DC therapy (“Combo (N=38”)) or with everolimus alone (“SOC (N=11)”). FIG. 14C shows Kaplan-Meier analysis of overall survival in the Phase 3 ADAPT trial (including everolimus as subsequent treatment in the follow up phase). The everolimus treated population included all mRCC patients receiving DC therapy in combination with everolimus (“Combo (N=22”)) or those who were treated with everolimus alone (“SOC (N=20)”). FIG. 14D shows a comparison of survival curves between subjects who received everolimus during follow up (N=22) or during treatment phase (N=38) of the study in combination therapy. FIG. 14E shows a comparison of survival curves between subjects who received everolimus during follow up (N=20) or during treatment phase (N=11) of the study in the SOC arm of the study. The median overall survival (OS) duration, a Hazard Ratio including 95% Confidence Interval (CI) range, and percent (%) censored value are shown.

FIGS. 15A-15D show T cell proliferation induced with DC stimulation. The frequency of Ki-67⁺ expression of CD4⁺ and CD4⁻ T cells was determined by flow cytometry for healthy donor (HD) PBMCs stimulated with autologous PME-CD40L-CMV DCs in the absence of plasma (“no plasma”) (FIG. 15A), in the presence of HD plasma (“HD plasma”) (FIG. 15B), or mRCC patient plasma (“mRCC plasma”) (FIG. 15C), in accordance with Example 7. FIG. 15D shows the frequency of CD4⁺ and CD4⁻ T cells (percent (%)) determined for two individual plasma samples collected from healthy donors (“healthy donor 1 plasma” and “healthy donor 2 plasma”), no plasma sample, and mRCC patient plasma treated sample. Data shown is from three independent experiments tested in duplicate. CD4 negative (CD4⁻) gating represents the CD8⁺ T cell population.

FIGS. 16A-16E show the effect of mRCC patient plasma on a mixed lymphocyte reaction (MLR) and OKT3 stimulation of T cell proliferation. The frequency of Ki-67⁺ expression of CD4⁺ and CD4⁻ T cells was determined for no plasma addition (left panel) and mRCC patient plasma treated (right panel) samples in a two-way MLR (FIG. 16A), in accordance with Example 7. The frequency of Ki-67⁺ expression of CD4⁺ and CD4⁻ T cells was determined by flow cytometry for no plasma addition (left panel) and mRCC patient plasma treated (right panel) samples in which OKT3 antibody was added to PMBC cultures (FIG. 16B), in accordance with Example 7. FIG. 16C shows the frequency of CD4⁺ and CD4⁻ T cells (percent of CD3 (%)) determined for two replicate cultures (MLR untreated; MLR mRCC patient plasma treated; OKT3 untreated; and OKT3 mRCC patient plasma treated), in accordance with Example 7. FIG. 16D shows CD25 expression level determined by measuring the geometric mean fluorescence intensity (MFI) of Ki-67⁺ cells for PMBCs stimulated in the MLR or with OKT3 antibody), in accordance with Example 7. FIG. 16E shows CD28 expression level determined by measuring the geometric mean fluorescence intensity (MFI) of Ki-67⁺ cells for PMBCs stimulated in the MLR or with OKT3 antibody, in accordance with Example 7. Data shown is from two independent experiments tested in duplicate. CD4 negative (CD4⁻) gating represents the CD8⁺ T cell population.

FIGS. 17A-17F show the effect of mRCC patient plasma and everolimus on healthy donor PBMC T cells stimulated in a MLR culture. The frequency of Ki-67⁺CD28⁺ expression of CD4⁺ (FIG. 17A) and CD8⁺ T cells (FIG. 17B) was determined for cultures untreated (left panels) or treated with mRCC patient plasma without (middle panels) or with the addition of everolimus (right panels), in accordance with Example 7. Cultures were performed in duplicate and a representative analysis is shown. Cumulative data collected from analysis of plasma samples collected from six individual mRCC patients tested in the MLR cultures show the frequencies (%) of Ki-67⁺CD4⁺CD28⁺(FIG. 17C), Ki-67⁺CD4⁺CD28⁻(FIG. 17D) T cells, Ki-67⁺CD8⁺CD28⁺ (FIG. 17E), and Ki-67⁺CD8⁺CD28⁻ (FIG. 17F) T cells untreated (“no plasma”) or treated with mRCC plasma with or without the addition of everolimus, in accordance with Example 7. Statistical significance was determined by student TTEST. *p value <0.05, **p value <0.001 and ns=not significant.

FIGS. 18A-18F show the effect of mRCC patient plasma and everolimus on healthy donor PBMC T cells stimulated with autologous PMECD40L-CMV DCs. The frequency of Ki-67⁺CD28⁺ expression of CD4⁺ (FIG. 18A) and CD8⁺ T cells (FIG. 18B) was determined for cultures untreated (left panels) or treated with mRCC patient plasma without (middle panels) or with the addition of everolimus (right panels), in accordance with Example 7. Cultures were performed in duplicate and a representative analysis is shown. Cumulative data collected from analysis of plasma samples collected from six individual mRCC patients tested are shown for the frequencies (%) of Ki-67⁺CD4⁺CD28⁺(FIG. 18C), Ki-67⁺CD4⁺CD28⁻(FIG. 18D) T cells, Ki-67⁺CD8⁺CD28⁺ (FIG. 17E), and Ki-67⁺CD8⁺CD28⁻ (FIG. 18F) T cells untreated (“no plasma”) or treated with mRCC plasma with or without the addition of everolimus, in accordance with Example 7. Statistical significance was determined by student TTEST. *p value <0.05, **p value <0.001 and ns=not significant.

FIGS. 19A-19F show effect of mRCC patient plasma and everolimus on healthy donor PBMC frequency of CD25⁺CD28⁺ T cells stimulated in a MLR culture. The frequency of CD25⁺CD28⁺ expression of CD4⁺ (FIG. 19A) and CD8⁺ T cells (FIG. 19B) was determined for cultures untreated (left panels) or treated with mRCC patient plasma without (middle panels) or with the addition of everolimus (right panels), in accordance with Example 8. Cultures were performed in duplicate and a representative analysis is shown. Cumulative data collected from analysis of plasma samples collected from six individual mRCC patients tested are shown for the frequencies (%) of CD25⁺/CD28⁺ CD4 T cells (FIG. 19C), CD25⁺/CD28⁻ CD8 T cells (FIG. 19D), and CD25⁺/CD28⁺ CD8 T cells (FIG. 19E), untreated (“no plasma”) or treated with mRCC plasma with or without the addition of everolimus, in accordance with Example 8. Statistical significance was determined by student TTEST. *p value <0.05, **p value <0.001 and ns=not significant.

FIGS. 20A-20E show the effect of mRCC patient plasma and everolimus on healthy donor PBMC T cells stimulated with autologous PMECD40L-CMV DCs. The frequency of Ki-67⁺CD28⁺ expression of CD4⁺ (FIG. 20A) and CD8⁺ T cells (FIG. 20B) was determined for cultures untreated (left panels) or treated with mRCC patient plasma without (middle panels) or with the addition of everolimus (right panels), in accordance with Example 8. Cultures were performed in duplicate and a representative analysis is shown. Cumulative data collected from analysis of plasma samples collected from six individual mRCC patients tested are shown for the frequencies of CD25⁺/CD28⁺ CD4 T cells (FIG. 20C), CD25⁺/CD28⁻ CD8 T cells (FIG. 20D), and CD25⁺/CD28⁺ CD8 T cells (FIG. 20E) and treated with mRCC plasma with or out without the addition of everolimus, in accordance with Example 8. Statistical significance was determined by student TTEST. *p value <0.05, **p value <0.001 and ns=not significant.

FIGS. 21A-21D show the effect of mRCC patient plasma and everolimus on expression of CD25 and CD28 on stimulated T cells. T cell responses were measured after stimulating HLA-mismatched healthy donor PBMCs in a MLR (FIG. 21A, FIG. 21B) or with autologous DCs (FIG. 21C, FIG. 21D) and cultured untreated (“no plasma”) or in the presence of mRCC patient plasma with or without everolimus, in accordance with Example 8. Cumulative data were collected from analysis of plasma samples collected from six individual mRCC patients tested. Expression levels of CD25 (FIG. 21A, FIG. 21C) and CD28 (FIG. 21B, FIG. 21D) were determined by measuring the mean fluorescence intensity (MFI) of Ki-67⁺ CD8 T cells. Statistical significance was determined by student TTEST. *p value <0.05, **p value <0.001 and ns=not significant.

FIGS. 22A-22B show TGF-β plasma levels in healthy donors (HD) and mRCC patients. FIG. 22A shows TGF-β levels (ng/mL) in the plasma collected from mRCC patients (mRCC n=8) or healthy donors (HD n=2), determined in accordance with Example 9. FIG. 22B shows the frequency (%) of proliferating (Ki-67⁺) CD4 T cells in MLR cultures, determined in accordance with Example 9. Data represents analysis of proliferative data obtained from treating MLR cultures with plasma obtained from six mRCC patients. Data are plotted versus the concentration of TGF-β detect in the plasma. Rho value is from the trend line calculated using excel software.

FIGS. 23A-23D show the effect of everolimus on TGF-β secretion and the frequency of CD38⁺ B cells from mRCC patients. FIG. 23A shows TGF-β (pg/mL) secreted by PBMCs from mRCC patients that were stimulated (“stim”) or left unstimulated (“control”) with or without everolimus addition as described in Example 9. FIG. 23B shows the frequency of CD38⁺ B cells determined by flow cytometry by gating on the viable CD19⁺ B cells in unstimulated cultures (“control”) or in stimulated cultures (“stim”) with or without everolimus addition as described in Example 9. Data are from three individual patient PBMCs analyzed. FIG. 23C shows the frequency of LAP⁺ /CD19⁺ B cells determined by flow cytometry in cultures stimulated without everolimus (upper panels) or with everolimus (lower panels) in accordance with Example 9. The gates to determine specific staining of the anti-LAP antibody was set by using an FMO (fluorescence minus one) gate (left panels). Positive staining was determined by setting gates on the CD19⁺/LAP⁺ cells (right panels). FIG. 23D shows the CD19⁺/LAP⁺ B cells (upper right quadrants in the right panels of FIG. 23C were further subgated to determine the frequency of IgG⁺ and CD38⁺ B cells). Quadrant gating identifies the distribution of IgG⁺CD38⁻ B cells (upper left quadrant), IgG⁺CD38⁺ B cells (upper right quadrant), IgG⁻CD38⁻ B cells (lower left quadrant) and IgG⁻CD3S⁺ B cells (lower right quadrant). Data are representative of three independent experiments from four individual mRCC patient PBMCs.

### DETAILED DESCRIPTION OF THE DISCLOSURE

Disclosed herein are methods of treating a tumor (e.g., renal cell cancer) by administering an immunotherapy comprising dendritic cells loaded with RNA encoding a tumor antigen and a pharmaceutical which can decrease circulating IgG levels, block IgG-mediated activation of CD16⁺ T cells, decrease the concentration or function of B cells reduce the frequency of CD38⁺ TGF-β⁺ B cells, decrease B cell secretion of TGF-β, and/or sustain the frequency of CD25⁺CD28⁺ CD4 and/or CD8 T cells.

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

As used herein, the terms “comprises”, “comprising”, “includes”, “including”, “having,” and their conjugates mean “including but not limited to.”

As used herein, the term “consisting of” means “including and limited to.”

As used herein, the term “consisting essentially of” means the specified material of a composition, or the specified steps of a method, and those additional materials or steps that do not materially affect the basic characteristics of the material or method.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, nucleotide sequences are written left to right in 5' to 3' orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

The terms “antibody” and “antibodies” are terms of art and can be used interchangeably herein and refer to an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. For example, the 3G8 antibody, the B73.1 antibody, or the CB16 antibody as disclosed herein are the anti-human CD16 antibodies.

An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “antibody fragment” refers to a portion of an intact antibody. An “antigen-binding fragment,” “antigen-binding domain,” or “antigen-binding region,” refers to a portion of an intact antibody that binds to an antigen. An antigen-binding fragment can contain an antigen recognition site of an intact antibody (e.g., complementarity determining regions (CDRs) sufficient to bind antigen). Examples of antigen-binding fragments of antibodies include, but are not limited to Fab, Fab', F(ab')2, and Fv fragments, linear antibodies, and single chain antibodies. An antigen-binding fragment of an antibody can be derived from any animal species, such as rodents (e.g., mouse, rat, or hamster) and humans or can be artificially produced.

The term “antigen” is well understood in the art and includes substances which are immunogenic, i.e., immunogen. It will be appreciated that the use of any antigen is envisioned for use in the present disclosure and thus includes, but is not limited to a self-antigen (whether normal or disease-related), an infectious antigen (e.g., a microbial antigen, viral antigen, etc.), or some other foreign antigen (e.g., a food component, pollen, etc.). The term “antigen” or alternatively, “immunogen” applies to collections of more than one immunogen, so that immune responses to multiple immunogens can be modulated simultaneously. Moreover, the term includes any of a variety of different formulations of immunogen or antigen. Furthermore, the antigen can be from a cancer cell (e.g., a renal cancer cell, a multiple myeloma cell, and a melanoma cell) or a pathogen (e.g., HIV and HCV). The antigen can be delivered to the antigen presenting cell (APC) in the form of RNA isolated or derived from a cancer cell or a pathogen. “Derived from” includes, but is not limited recombinant variants of naturally occurring sequences, including fusions to unrelated or related sequences. Methods for RT-PCR of RNA extracted from any cell (e.g., a cancer cell or pathogen cell), and in vitro transcription are disclosed, for example in PCT/US05/053271.

A “native” or “natural” or “wild-type” antigen is a polypeptide, protein or a fragment which contains an epitope, which has been isolated from a natural biological source, and which can specifically bind to an antigen receptor, when presented as an MHC/peptide complex, in particular a T cell antigen receptor (TCR), in a subject.

As used herein, “tumor antigen” or “tumor associated antigen” or “TAA” refers to an antigen that is associated with a tumor. Examples of well-known TAAs include gp100, melanoma-associated antigen recognized by T cells (“MART”) and melanoma-associated antigen (“MAGE”).

As used herein, an “epitope” is a term in the art and refers to a localized region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non- linear, discontinuous, or non-contiguous epitope). Epitopes formed from contiguous amino acids are typically, but not always, retained on exposure to denaturing solvents, whereas epitopes formed by tertiary folding are typically lost on treatment with denaturing solvents. An epitope typically includes at least 1, 2, 3, 4, 5, 6, 7, 8, 9, 10, 11, 12, 13, 14, 15, or 20 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from (e.g., CD16) are tested for reactivity with a given antibody (e.g., anti-human CD16 antibody). Methods of determining spatial conformation of epitopes include techniques in the art and those described herein, for example, x-ray crystallography, 2-dimensional nuclear magnetic resonance and HDX-MS (see, e.g., Epitope Mapping Protocols in Methods in Molecular Biology, Vol. 66, G. E. Morris, Ed. (1996)).

In certain aspects, the epitope to which an antibody binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization can be accomplished using any of the known methods in the art (e.g., Giege R et al., (1994) Acta Crystallogr D Biol Crystallogr 50(Pt 4): 339-350; McPherson A (1990) EurJBiochem 189: 1-23; Chayen NE (1997) Structure 5: 1269-1274; McPherson A (1976) J Biol Chem 251 : 6300-6303). Antibody:ant+igen crystals can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff HW et al.,; U.S. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter CW; Roversi P et al., (2000) Acta Crystallogr D Biol Crystallogr 56(Pt 10): 1316-1323). Mutagenesis mapping studies can be accomplished using any method known to one of skill in the art. See, e.g., Champe M et al., (1995) J Biol Chem 270: 1388-1394 and Cunningham BC & Wells JA (1989) Science 244: 1081- 1085 for a description of mutagenesis techniques, including alanine scanning mutagenesis techniques.

The term “epitope mapping” refers to the process of identification of the molecular determinants for antibody-antigen recognition.

The term “binds to the same epitope” as a reference antibody means that the antibodies bind to the same segment of amino acid residues, as determined by a given method. Techniques for determining whether antibodies bind to the “same epitope on CD16” with the antibodies described herein include, for example, epitope mapping methods, such as, x-ray analyses of crystals of antigen:antibody complexes which provides atomic resolution of the epitope and hydrogen/deuterium exchange mass spectrometry (HDX-MS). Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, 2 and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, can be determined using known competition experiments, e.g., BIACORE® surface plasmon resonance (SPR) analysis. In some aspects, an antibody competes with, and inhibits binding of another antibody to a target by at least 50%, 60%, 70%, 80%, 90% or 100%. The level of inhibition or competition can be different depending on which antibody is the “blocking antibody” (i.e., the cold antibody that is incubated first with the target). Competition assays can be conducted as described, for example, in Ed Harlow and David Lane, Cold Spring Harb Protoc; 2006; doi: 10.1101/pdb.prot4277 or in Chapter 11 of “Using Antibodies” by Ed Harlow and David Lane, Cold Spring Harbor Laboratory Press, Cold Spring Harbor, NY, USA 1999. Two antibodies “cross-compete” if antibodies block each other both ways by at least 50%, i.e., regardless of whether one or the other antibody is contacted first with the antigen in the competition experiment.

Competitive binding assays for determining whether two antibodies compete or cross-compete for binding include: competition for binding to cells expressing CD16, e.g., by flow cytometry, such as described in the Examples. Other methods include: SPR (e.g., BIACORE®), BLI (Bio-layer interferometry), solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)).

The terms “major histocompatibility complex” or “MHC” refers to a complex of genes encoding cell-surface molecules that are required for antigen presentation to T cells and for rapid graft rejection. In humans, the MHC is also known as the “human leukocyte antigen” or “HLA” complex. The proteins encoded by the MHC are known as “MHC molecules” and are classified into Class I and Class II MHC molecules. Class I MHC molecules include membrane heterodimeric proteins made up of an α chain encoded in the MHC noncovalently linked with the (β₂-microglobulin. Class I MHC molecules are expressed by nearly all nucleated cells and have been shown to function in antigen presentation to CD8⁺ T cells. Class I molecules include HLA-A, B, and C in humans. Class II MHC molecules also include membrane heterodimeric proteins consisting of noncovalently associated α and β chains. Class II MHC molecules are known to function in CD4⁺ T cells and, in humans, include HLA-DP, -DQ, and -DR.

As used herein, the term “cytokine” refers to any one of the numerous factors that exert a variety of effects on cells, for example, inducing growth or proliferation. Nonlimiting examples of cytokines which can be used alone or in combination in the practice of the present invention include, interleukin-2 (IL-2), stem cell factor (SCF), interleukin-3 (IL-3), interleukin-4 (IL-4), interleukin-6 (IL-6), interleukin-11 (IL-11), interleukin-12 (IL-12), interleukin-13 (IL-13), interleukin-15 (IL-15), granulocyte-colony stimulating factor (G-CSF), granulocyte macrophage-colony stimulating factor (GM-CSF), interleukin-1 beta (IL-1 β), interferon-γ (IFNγ), tumor necrosis factor-α (TNFα), prostaglandin E₂ (PGE₂), MIP-11, leukemia inhibitory factor (LIF), c-kit ligand, thrombopoietin (TPO) and flt3 ligand. Cytokines are commercially available from several vendors such as, for example, Genzyme (Framingham, Mass.), Genentech (South San Francisco, Calif.), Amgen (Thousand Oaks, Calif.), R&D Systems (Minneapolis, Minn.) and Immunex (Seattle, Wash.). It is intended, although not always explicitly stated, that molecules having similar biological activity as wild-type or purified cytokines (e.g., recombinantly produced or muteins thereof) are intended to be used within the spirit and scope of the disclosure.

The term “antigen presenting cells (APCs)” refers to a class of cells capable of presenting one or more antigens in the form of peptide-MHC complex recognizable by specific effector cells of the immune system, and thereby inducing an effective cellular immune response against the antigen or antigens being presented. APCs can be intact whole cells such as macrophages, B-cells, endothelial cells, activated T-cells, and dendritic cells; or other molecules, naturally occurring or synthetic, such as purified MHC Class I molecules complexed to β2-microglobulin. While many types of cells may be capable of presenting antigens on their cell surface for T-cell recognition, only dendritic cells have the capacity to present antigens in an efficient amount to activate naive T-cells for cytotoxic T-lymphocyte (CTL) responses.

The term “dendritic cells (DCs)” refers to a diverse population of morphologically similar cell types found in a variety of lymphoid and non-lymphoid tissues, Steinman et al., Ann. Rev. Immunol. 9:271-296 (1991). Dendritic cells constitute the most potent and preferred APCs in the organism. While the dendritic cells can be differentiated from monocytes, they possess distinct phenotypes. For example, a particular differentiating marker, CD14 antigen, is not found in dendritic cells but is possessed by monocytes. Also, mature dendritic cells are not phagocytic, whereas the monocytes are strongly phagocytosing cells. It has been shown that mature DCs can provide all the signals necessary for T cell activation and proliferation. Immature DCs are capable of capturing antigens by endocytosis, phagocytosis, macropinocytosis or adsorptive pinocytosis and receptor mediated antigen uptake, and are phenotypically CD80⁻ or CD80^{low}, CD83⁻ or CD83^{low}, CD86^{low}, and have high intracellular concentrations of MHC class II molecules. Mature DCs have a veiled morphology, a lower capacity for endocytosis and are phenotypically CD80^{high}, CD83^{high}, CD86^{high} in comparison to immature DCs. The mature DCs secrete IL-12 p70 polypeptide or protein, and/or secrete significantly reduced levels (0 to 500 pg/ml per million DCs) of IL-10. IL-10 and IL-12 levels can be determined by ELISA of culture supernatants collected at up to 36 hrs post induction of DC maturation from immature DCs. Wierda W. G. et al., Blood 96: 2917 (2000); Ajdary S et al., Infection and Immunity 68:1760 (2000); Banchereau and Steinman et al., Nature 392:245 (1998).

The term “B cell” or “B-cell” or “B lymphocytes” refers to a lymphocyte, a type of white blood cell (leukocyte), that develops into a plasma cell (a “mature B cell”), which produces antibodies. An “immature B cell” is a cell that can develop into a mature B cell. Generally, pro-B cells (that express, for example, CD45 or B220) undergo immunoglobulin heavy chain rearrangement to become pro B pre B cells, and further undergo immunoglobulin light chain rearrangement to become immature B cells. Immature B cells can develop into mature B cells, which can produce immunoglobulins (e.g., IgA, IgG or IgM). Mature B cells express characteristic markers such as CD21 and CD23 (CD23^{hi}CD21^{hi} cells). B cells can be activated by agents such as lippopolysaccharide (LPS) or IL-4 and antibodies to IgM. See e.g., U.S. Appl. Publ. No. US 2012/0308563.

As used herein, the phrase “decreasing the concentration of B cells” or “decrease the concentration of B cells” refers to decreasing the number of B cells. In some aspects, administering the pharmaceutical as described herein can decrease the concentration of B cells by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90% in e.g., mRCC patient’s peripheral blood mononuclear cells (PBMCs) compared to the number of B cells measured in the absence of the pharmaceutical.

As used herein, the phrase “decrease B cell secretion of TGF-0” refers to a decrease in the concentration of TGF-β secreted by B cells (e.g., stimulated B cells). In some aspects, administering the pharmaceutical as described herein can decrease the concentration of TGF-β by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, or at least about 90%, e.g., as measured in the plasma of mRCC patients compared to the concentration of TGF-β measured in the absence of the pharmaceutical.

As used herein, the phrase “reduce the frequency of CD38⁺ TGF-β⁺ B cells” refers to decreasing the number of CD38⁺ TGF-β⁺ B cells. In some aspects, administering the pharmaceutical as described herein can decrease the number of CD38⁺ TGF-β⁺ B cells by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, or at least about 90%, in e.g., mRCC patient’s peripheral blood mononuclear cells (PBMCs) compared to the number of B cells measured in the absence of the pharmaceutical. In some aspects, the pharmaceutical (e.g., everolimus) can modulate the ability of B cells to differentiate into CD38⁺ TGF-β⁺ B cells.

As used herein, the phrase “decreasing the function of B cells” refers to, for example, decreased B cell proliferation, downregulation of the expression of CD80 and CD86, decreased level of immunoglobulin (e.g., IgG and IgM) production, and/or decreased level or cytokine (e.g., IL-10) production. Matz et al., European Society for Organ Transplantation 25: 1106-1116 (2012).

As used herein, the phrase “sustain the frequency of CD25⁺CD28⁺ CD4 and/or CD8 T cells” refers to increasing the number of CD25⁺CD28⁺ CD4 and/or CD8 T cells. In some aspects, administering the pharmaceutical as described herein can increase the number of CD25⁺CD28⁺ CD4 and/or CD8 T by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, or at least about 90%, in e.g., mRCC patient’s peripheral blood mononuclear cells (PBMCs) compared to the number of B cells measured in the absence of the pharmaceutical.

As used herein, the phrase “decreasing circulating IgG levels” refers to decreased IgG production. Representing approximately 75% of serum antibodies in humans, IgG is the most common type of antibody found in blood circulation. Junqueira et al., Basic Histology. McGraw-Hill (2003). IgG molecules are created and released by plasma B cells. In some aspects, administering the pharmaceutical as described herein can decrease circulating IgG levels in a patient’s serum and/or plasma by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90% compared to circulating IgG levels measured in the absence of the pharmaceutical.

The term “immune effector cells” refers to cells capable of binding an antigen and which mediate an immune response. These cells include, but are not limited to, T cells, B cells, monocytes, macrophages, NK cells and cytotoxic T lymphocytes (CTLs), for example CTL lines, CTL clones, and CTLs from tumor, inflammatory, or other infiltrates.

A “naive” immune effector cell is an immune effector cell that has never been exposed to an antigen capable of activating that cell. Activation of naive immune effector cells requires both recognition of the peptide:MHC complex and the simultaneous delivery of a costimulatory signal by a professional APC in order to proliferate and differentiate into antigen-specific armed effector T cells.

An “immune response” is as understood in the art, and generally refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate’s body of invading pathogens, cells or tissues infected with pathogens, cancerous or other abnormal cells, or, in cases of Autoimmunity or pathological inflammation, normal human cells or tissues. An immune reaction includes, e.g., activation or inhibition of a T cell, e.g., an effector T cell, a Th cell, a CD4⁺ cell, a CD8⁺ T cell, or a Treg cell, or activation or inhibition of any other cell of the immune system, e.g., NK cell.

As used herein, the term “educated, antigen-specific immune effector cell,” is an immune effector cell as defined above, which has previously encountered an antigen. In contrast to its naive counterpart, activation of an educated, antigen specific immune effector cell does not require a costimulatory signal. Recognition of the peptide: MHC complex is sufficient.

“Activated,” when used in reference to a T cell, implies that the cell is no longer in G₀ phase, and begins to produce one or more of cytotoxins, cytokines and other related membrane-associated proteins characteristic of the cell type (e.g., CD8⁺ or CD4⁺), and is capable of recognizing and binding any target cell that displays the particular peptide/MHC complex on its surface, and releasing its effector molecules.

“Immunotherapy” refers to the treatment of a subject afflicted with, or at risk of contracting or suffering a recurrence of, a disease by a method comprising inducing, enhancing, suppressing or otherwise modifying the immune system or an immune response. For example, CMN-001 is an autologous immunotherapy prepared from fully matured and optimized monocyte-derived dendritic cells, which are co-electroporated with amplified tumor RNA plus synthetic CD40L RNA, as described in U.S. Pat. No. 8,822,223, which is herein incorporated by reference and infra in the Examples.

“Immuno stimulating therapy” or “immuno stimulatory therapy” refers to a therapy that results in increasing (inducing or enhancing) an immune response in a subject for, e.g., treating cancer.

“T effector” (“Teff”) cells refer to T cells (e.g., CD4+ and CD8+ T cells) with cytolytic activities as well as T helper (Th) cells, e.g., Th1 cells, which cells secrete cytokines and activate and direct other immune cells, but does not include regulatory T cells (Treg cells).

“T regulatory” (“Treg”) cells refer to T cells that modulate the immune system, maintain tolerance to self-antigens, and prevent autoimmune disease. Tregs are immunosuppressive and generally suppress or downregulate induction and proliferation of effector T cells. Bettelli et al., Nature 441(7090): 235-238 (2006). Tregs express the biomarkers CD4, FOXP3, and CD25 and are thought to be derived from the same lineage as naïve CD4 cells. Curiel T.J., The Journal of Clinical Investigation. 117(5):1167-1174 (2007). TGFβ is essential for Tregs to differentiate from naive CD4+ cells and is important in maintaining Treg homeostasis. Chenet W. Immunotherapy 3(8): 911-914 (2011).

An increased ability to stimulate an immune response or the immune system, can result from an enhanced agonist activity of T cell co-stimulatory receptors and/or an enhanced antagonist activity of inhibitory receptors. An increased ability to stimulate an immune response or the immune system can be reflected by a fold increase of the EC50 or maximal level of activity in an assay that measures an immune response, e.g., an assay that measures changes in cytokine or chemokine release, cytolytic activity (determined directly on target cells or indirectly via detecting CD107a or granzymes) and proliferation. The ability to stimulate an immune response or the immune system activity can be enhanced by at least 10%, 30%, 50%, 75%, 2 fold, 3 fold, 5 fold or more.

As used herein, the term “T cell-mediated response” refers to a response mediated by T cells, including effector T cells (e.g., CD8+ cells) and helper T cells (e.g., CD4+ cells). T cell mediated responses include, for example, T cell cytotoxicity and proliferation.

As used herein, the term “cytotoxic T lymphocyte (CTL) response” refers to an immune response induced by cytotoxic T cells. CTL responses are mediated primarily by CD8+ T cells.

As used herein, the terms “inhibits” or “blocks” (e.g., referring to inhibition/blocking of IgG-mediated activation of CD16⁺ T cells) are used interchangeably and encompass both partial and complete inhibition/blocking. In some aspects, an anti-human CD16 antibody (e.g., the 3G8 antibody, the B73.1 antibody, or the CB16 antibody) inhibits binding of IgG to the low affinity Fc receptor, CD16, by at least about 50%, for example, about 60%, 70%, 80%, 90%, 95%, 99%, or 100%, determined, e.g., as further described herein.

The term “autologous” refers to any material derived from the same individual to which it is later to be re-introduced. For example, the tumor antigen that is autologous to the patient comprises administering to a subject the tumor antigen that was isolated from the same subject.

A “cancer” refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. An example of a cancer that can be treated by the methods of the present disclosure includes, but is not limited to, renal cell cancer. In some aspects, the methods of the present disclosure can be used to reduce the tumor size of a tumor derived from, for example, renal cancer, breast cancer, pancreatic cancer, brain cancer (e.g., astrocytoma, glioblastoma multiforme), bone cancer, prostate cancer, colon cancer, lung cancer, cutaneous or intraocular malignant melanoma, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Waldenstrom macroglobulinaemia, Hodgkin’s Disease, non-Hodgkin’s lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia (AML), chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, pituitary adenoma, Kaposi’s sarcoma, epidermoid cancer, squamous cell cancer, T-cell lymphoma, environmentally induced cancers including those induced by asbestos, other B cell malignancies, and combinations of said cancers.

The term “tumor” as used herein refers to any mass of tissue that results from excessive cell growth or proliferation, either benign (non-cancerous) or malignant (cancerous), including pre-cancerous lesions.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease or enhancing overall survival. Treatment can be of a subject having a disease or a subject who does not have a disease (e.g., for prophylaxis).

“Disease progression” or “progressive disease,” which can be abbreviated as PD, as used herein, refers to a worsening of one or more symptom associated with a particular disease. For example, disease progression for a patient having a tumor (e.g., tumor progression) can include an increase in the number or size of one or more malignant lesions, tumor metastasis, and death.

The term “effective dose” or “effective dosage” or “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to delay other unwanted cell proliferation. In some aspects, an effective amount is an amount sufficient to prevent or delay tumor recurrence. An effective amount can be administered in one or more administrations. The effective amount of the drug or composition can: (i) reduce the number of cancer cells; (ii) reduce tumor size; (iii) inhibit, retard, slow to some extent and can stop cancer cell infiltration into peripheral organs; (iv) inhibit (i.e., slow to some extent and can stop tumor metastasis; (v) inhibit tumor growth; (vi) prevent or delay occurrence and/or recurrence of tumor; and/or (vii) relieve to some extent one or more of the symptoms associated with the cancer.

By way of example for the treatment of tumors, a therapeutically effective amount or dosage of the drug inhibits cell growth or tumor growth by at least about 20%, by at least about 40%, by at least about 60%, or by at least about 80% relative to untreated subjects. In some aspects, a therapeutically effective amount or dosage of the drug completely inhibits cell growth or tumor growth, i.e., inhibits cell growth or tumor growth by 100%. The ability of a compound to inhibit tumor growth can be evaluated using the assays described infra. Alternatively, this property of a composition can be evaluated by examining the ability of the compound to inhibit cell growth, such inhibition can be measured in vitro by assays known to the skilled practitioner. In some aspects described herein, tumor regression can be observed and continue for a period of at least about 20 days, at least about 40 days, or at least about 60 days.

The term “weight based” dose or dosing as referred to herein means that a dose that is administered to a patient is calculated based on the weight of the patient. For example, when a patient with 60 kg body weight requires 3 mg/kg of an anti-CD16 antibody, one can calculate and use the appropriate amount of the anti-CD16 antibody (i.e., 180 mg) for administration.

The use of the term “flat dose” with regard to the methods and dosages described herein means a dose that is administered to a patient without regard for the weight or body surface area (BSA) of the patient. The flat dose is therefore not provided as a mg/kg dose, but rather as an absolute amount of the agent (e.g., the anti-CD16 antibody). For example, a 60 kg person and a 100 kg person would receive the same dose of an antibody (e.g., 480 mg of an anti-CD16 antibody).

As used herein, the term a “composition” refers to a combination of active agent and another compound or composition, inert (for example, a detectable agent or label) or active, such as an adjuvant.

As used herein, the term a “pharmaceutical composition” as described herein refers to the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin REMINGTON’S PHARM. SCI., 18th Ed. (Mack Publ. Co., Easton (1990)).

As used herein, “administering” refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some aspects, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

A “dose regimen” as used herein, is the manner in which an immunotherapy or a pharmaceutical is administered (e.g., the magnitude of each dose (dose size), the frequency and interval with which the dose is repeated.

“Dosing interval,” as used herein, means the amount of time that elapses between multiple doses of e.g., an immunotherapy or a pharmaceutical disclosed herein being administered to a subject. Dosing interval can thus be indicated as ranges.

The term “dosing frequency” as used herein refers to the frequency of administering doses of e.g., an immunotherapy or a pharmaceutical disclosed herein in a given time. Dosing frequency can be indicated as the number of doses per a given time, e.g., once per day or once per week or once every three weeks.

The terms “about once a week,” “once about every week,” “once about every two weeks,” or any other similar dosing interval terms as used herein means approximate number, and “about once a week” or “once about every week” can include every seven days ± two days, i.e., every five days to every nine days. The dosing interval of “once a week” thus can be every five days, every six days, every seven days, every eight days, or every nine days. “Once about every two weeks” can include every fourteen days ± three days, i.e., every eleven days to every seventeen days. Similar approximations apply, for example, to once about every three weeks, once about every four weeks, once about every five weeks, once about every six weeks and once about every twelve weeks. In some aspects, a dosing interval of once about every six weeks or once about every twelve weeks means that the first dose can be administered any day in the first week, and then the next dose can be administered any day in the sixth or twelfth week, respectively. In other aspects, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

A “patient” as used herein includes any human who is afflicted with a tumor (e.g., mRCC). The terms “subject” and “patient” are used interchangeably herein.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein’s or peptide’s sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The terms “polynucleotide,” “nucleic acid” and “nucleic acid molecule” are used interchangeably to refer to polymeric forms of nucleotides of any length. The polynucleotides may contain deoxyribonucleotides, ribonucleotides, and/or their analogs. Nucleotides can have any three-dimensional structure, and can perform any function, known or unknown. The term “polynucleotide” includes, for example, single-stranded, double-stranded and triple helical molecules, a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. In addition to a native nucleic acid molecule, a nucleic acid molecule of the present disclosure can also comprise modified nucleic acid molecules. As used herein, mRNA refers to an RNA that can be translated in a dendritic cell. Such mRNAs typically are capped and have a ribosome binding site (Kozak sequence) and a translational initiation codon.

As used herein, the terms “ug” and “uM” are used interchangeably with “µg” and “µM,” respectively.

Various aspects described herein are described in further detail in the following subsections.

### II. Methods of the Disclosure

The present disclosure is directed to methods of treating diseases or conditions which comprise a tumor, comprising the sequential steps of: (a) administering a dose regimen of an immunotherapy comprising dendritic cells loaded with RNA encoding a tumor antigen to a patient having a tumor; and (b) after administration of at least one dose of the immunotherapy of step (a), administering a dose regimen of a pharmaceutical which can cause one or more of the following: (i) decrease circulating IgG levels, (ii) block IgG-mediated activation of CD16+T cells, (iii) decrease the concentration and/or function of B cells, (iv) reduce the frequency of CD38⁺ TGF-β⁺ B cells, (v) decrease B cell secretion of TGF-β, and (vi) sustain the frequency of CD25⁺CD28⁺ CD4 and/or CD8 T cells.

The present disclosure is also directed methods of decreasing circulating IgG levels, blocking IgG-mediated activation of CD16+ T cells, and/or decreasing the concentration or function of B cells in a patient having a tumor, comprising the sequential steps of: (a) administering a dose regimen of an immunotherapy comprising dendritic cells loaded with RNA encoding a tumor antigen to a patient having a tumor; and (b) after administration of at least one dose of the immunotherapy of step (a), administering a dose regimen of a pharmaceutical which can cause one or more of the following: (i) decrease circulating IgG levels, (ii) block IgG-mediated activation of CD16+ T cells, (iii) decrease the concentration and/or function of B cells, (iv) reduce the frequency of CD38⁺ TGF-β⁺ B cells, (v) decrease B cell secretion of TGF-β, and (vi) sustain the frequency of CD25⁺CD28⁺ CD4 and/or CD8 T cells.

### Immunotherapies

In one aspect, immunotherapies provided herein involve administration of a dose regimen of an immunotherapy comprising dendritic cells loaded with RNA encoding a tumor antigen to a patient having a tumor. In some aspects, dosage regimens are adjusted to provide the optimum desired response (e.g., an effective response).

As used herein, adjunctive or combined administration (co-administration) includes simultaneous administration of the immunotherapy and the pharmaceutical in the same or different dosage form, or separate administration of the immunotherapy and the pharmaceutical (e.g., sequential administration). Thus, for example, the immunotherapy comprising dendritic cells loaded with RNA encoding a tumor antigen and the pharmaceutical can be simultaneously administered. Alternatively, the immunotherapy comprising dendritic cells loaded with RNA encoding a tumor antigen and the pharmaceutical can be administered sequentially (e.g., the CMN-001 immunotherapy is intradermally injected at the same time or within about 30 seconds to about 10 minutes prior to oral administration of the pharmaceutical, or immediately before an intravenous administration of the pharmaceutical).

For example, the immunotherapy comprising dendritic cells loaded with RNA encoding a tumor antigen and the pharmaceutical can be administered first followed by (e.g., immediately followed by) the administration of the pharmaceutical. In some aspects, the immunotherapy and the pharmaceutical are administered concurrently. Such concurrent or sequential administration can result in both the immunotherapy and the pharmaceutical being simultaneously present in the treated subjects.

In some aspects, the immunotherapy is CMN-001, which is an autologous immunotherapy prepared from fully matured and optimized monocyte-derived dendritic cells, which are co-electroporated with amplified tumor RNA plus synthetic CD40L RNA (e.g., as described in Calderhead DM., et al. Keynote Symposia on Molecular and Cellular Biology, Vancouver, BC, Poster Presentation. Feb. 1-7, 2005; Calderhead DM., et al. J. Immunother. 31(8):731-741 (2008); DeBenedette MA. etal., J. Immunol. 181(8):5296-305 (2008); DeBenedette MA. et al., J. Immunother. 34(1):45-57 (2011) and in the Examples).

In certain aspects, CMN-001 is administered about once every week, about once every two weeks, about once every three weeks, about once every four weeks, about once every five weeks, about once every six weeks, about once every seven weeks, about once every eight weeks, about once every nine weeks, about once every ten weeks, about once every eleven weeks, or about once every twelve weeks. In certain aspects, CMN-001 is administered about once every three weeks. In some aspects, CMN-001 is administered once every three weeks.

In some aspects, the regimen of immunotherapy continues after the initiation of the dose regimen of the pharmaceutical. For example, CMN-001 immunotherapy can be administered about once every week, about once every two weeks, about once every three weeks, about once every four weeks, or about once quarterly. In some aspects, the mTOR inhibitors are orally administered about once daily anytime during CMN-001 immunotherapy administration. In some aspects, the mTOR inhibitors are intravenously administered about once every week along with the immunotherapy schedule.

### Pharmaceuticals

As used herein, the pharmaceuticals can cause one or more of the following: (i) decrease circulating IgG levels, (ii) block IgG-mediated activation of CD16+ T cells, (iii) decrease the concentration and/or function of B cells, (iv) reduce the frequency of CD38⁺ TGF-β⁺ B cells, (v) decrease B cell secretion of TGF-β, and (vi) sustain the frequency of CD25⁺CD28⁺ CD4 and/or CD8 T cells.

In certain aspects, the pharmaceuticals provided herein involve mammalian target of rapamycin (mTOR) inhibitors. In some aspects, the mTOR inhibitor is rapamycin or a rapamycin analog. In certain aspects, the rapamycin analog includes everolimus, temsirolimus, sirolimus, ridaforolimus, or combinations thereof.

The inhibitors, everolimus and temsirolimus target the serine/threonine protein mTOR, which results in a block in protein synthesis and loss of cellular proliferation. mTOR controls downstream cell cycle regulators in PI3K/Akt pathway of growth factor receptors. Downstream protein targets of mTOR include 4EBP1 and p70S6K, which influence angiogenesis, cell growth, translation, and protein synthesis (Liao, C. et al., Cancer 110: 1501-1508 (2007)). Functionally, mTOR exists as two complexes, mTORC1 and mTORC2. mTORC1, which is sensitive to rapamycin, everolimus and temsirolimus consists of, mTOR, PRAS40 (proline-rich AKT substrate 40 kDa), mLST8 (mammalian lethal with sec-13), and regulatory associated protein of mTOR (RAPTOR). Rapamycin complexes with intracellular receptor FKBP12 (FK506-binding protein of 12 kDa) and binds to the FRB domain of mTOR and inhibits downstream signaling through mTORC1 (Yip, C. et al., Mol Cell 38: 768-774 (2010); Mills, R. E. et al., Cell Cycle 8: 545-548 (2009); Foti et al., Clin Sci 129: 895-914 (2015)).

Everolimus has greater stability and enhanced solubility in organic solvents, as well as more favorable pharmokinetics with fewer side effects than rapamycin (sirolimus). See, e.g., U.S. Pub. No. 2012/0027757. It is marketed by Novartis, under the trade names Zortress® (USA) and Certican® (e.g., Europe) in transplantation medicine, and as Afinitor® (general tumors) and Votubia® (tumors as a result of tuberous sclerosis complex (“TSC”) in oncology. Everolimus is also available from Biocon, with the brand name Evertor.In certain aspects, everolimus is administered about once per day. In some aspects about 2.5 mg to about 20 mg, about 3 mg to about 19 mg, about 4 mg to about 18 mg, about 5 mg to about 17 mg, about 6 mg to about 16 mg, about 7 mg to about 15 mg, about 8 mg to about 14 mg, about 9 mg to about 13 mg, or about 10 mg to about 12 mg of everolimus is administered once per day. In some aspects about 2.5 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg of everolimus is administered once per day. In some aspects, about 10 mg of everolimus is administered once per day. In certain aspects, 10 mg of everolimus is administered once per day. In some aspects, about 5 mg of everolimus is administered once per day. In certain aspects, 5 mg of everolimus is administered once per day.

Temsirolimus, also known as Torisel®, is marketed by Pfizer Inc. for the treatment of renal cell carcinoma. A description and preparation of temsirolimus is described e.g., in U.S. Pat. No. 5,362,718.

In certain aspects, temsirolimus is administered about once per week. In some aspects about 2.5 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24, about 25 mg, about 26 mg, about 27 mg, about 28 mg, about 29 mg, or about 30 mg of temsirolimus is administered once per week. In some aspects, about 25 mg of temsirolimus is administered once per week. In some aspects, 25 mg of temsirolimus is administered once per week. In certain aspects, about 25 mg of temsirolimus is administered over a 30 to 60 minute period once a week. Sirolimus is marketed by Pfizer Inc., under the trade name Rapamune®.

Ridaforolimus, also known as AP 23573, MK-8669, and formerly known as deforolimus, is a unique, non-prodrug analog of rapmycin that has antiproliferative activity in a broad range of human tumor cell lines in vitro and in murine tumor xenograft models utilizing human tumor cell lines. Ridaforolimus has been administered to patients with advanced cancer. See, e.g., U.S. Pub. No. 2012/0027757. A description and preparation of ridaforolimus is described e.g., in in U.S. Pat. No. 7,091,213.

In certain aspects, a first dose of the mTOR inhibitor is administered after progression of the tumor. Progressive disease (e.g., progression of the tumor) is defined by the Response Evaluation Criteria in Solid Tumors (RECIST) 1.1 cirteria as described in e.g., Schwartz et al., Eur J Cancer 62: 132-137 (2016). In some aspects, progression of the tumor, e.g., target lesions increase in size or apprearance of new lesions is evident on a computed tomography (CT or CAT) scan.

The mTOR inhibitors of the present disclosure can also exist as various crystals, amorphous substances, pharmaceutically acceptable salts, hydrates, and solvates.

Further, the mTOR inhibitors of the present disclosure can be provided as prodrugs. In general, such prodrugs are functional derivatives of the mTOR inhibitors of the instant disclosure that can be readily converted into compounds that are needed by living bodies. Accordingly, in the methods of treating diseases or conditions which comprise a tumor (e.g., renal cell cancer) as described herein, the term “administration” includes not only the administration of a specific compound but also the administration of a compound which, after administered to patients, can be converted into the specific compound in the living bodies. Conventional methods for selection and production of suitable prodrug derivatives are described, for example, in “Design of Prodrugs,” ed. H. Bundgaard, Elsevier, 1985, which is referred to herein and is entirely incorporated herein as a part of the present description. Metabolites of the compound can include active compounds that are produced by putting the compound in a biological environment, and are within the scope of the compound in the disclosure.

In some aspects, the pharmaceutical decreasing the function of B cells is chosen from natalizumab (Tysabri®) (as described in e.g., Börnsen L et al., PLoS One. 7(11): e47578 (2010); Braley T. J. et al. Curr Treat Options Neurol. 15(3):259-269 (2013); teriflunomide (Aubagio®) (as described in e.g., Heinz W et. al., Abstract, Annual Meeting of the Consortium of Multiple Sclerosis Centers (CMSC), National Harbor, MD, USA, June 1-4, 2016; Klotz et. al., Science Translational Medicine 11(490): eaao5563 (2019); Braley T. J. et al. Curr Treat Options Neurol. 15(3):259-269 (2013)); or ofatumumab (Arzerra®) (as described in e.g., Vitale et. al., Clin Cancer Res; 22(10); 2359-2367 (2016)).

In some aspects, the pharmaceutical decreasing the concentration of B cells is chosen from prednisone (as described in e.g., Salinas-Carmona M. C. et al., Autoimmunity. 42(6):537-544 (2009); Settipane, G. A. et al., J Allergy Clin Immunol. 62(3):162-166 (1978); Agarwal et al., Ann Allergy Asthma Immunol. 99(3):281-283 (2007); Giles et al., J ImmunoTherapy Cancer 6(51) (published Jun. 11, 2018)); cyclophosphamide (as described in e.g., Salinas-Carmona M. C. et al., Autoimmunity. 42(6):537-544 (2009); Ahlmann et al., Cancer Chemother Pharmacol. (4):661-671(2016); Scurr et al., Clin Cancer Res. 23(22):6771-6780 (2015); Madondo et al., Cancer Treat Rev. 42:3-9 (2016)); methotrexate (as described in e.g., Salinas-Carmona M. C. et al., Autoimmunity. 42(6):537-544 (2009); Bulatovic et al., Rheumatology 54(9):1724-1734 (2015); Cribbs et al., Arthritis Rheumatol. (5):1182-1192 (2015); Yu et al., Clinical and Developmental Immunology, Volume 2013, Article ID 238035, 12 pages (2015)); mycophenolate mofetil (as described in e.g., Salinas-Carmona M. C. et al., Autoimmunity. 42(6):537-544 (2009); Ritter et al., Transpl Infect Dis. 11(4):290-297 (2009); Allison et al., Immunopharmacology 47(2-3):85-118 (2000); McMurray et al., Am J Med Sci. 323(4):194-196 (2002)); azathioprine (as described in e.g., Salinas-Carmona M. C. et al., Autoimmunity. 42(6):537-544 (2009); Leitner et al., Immunol Lett. 140(1-2):74-80 (2011); McCarthy et al., J Clin Invest. 125(8):3215-3225 (2015)); trimetrexate (as described in e.g., Rosenthal et al., J Immunol. 141(2):410-416 (1988); Mader et al., Comprehensive Medicinal Chemistry II , 7:55-79 (2007); Pappo et al., J Natl Cancer Inst. 82(20):1641-1642 (1990)); cortisol (as described in e.g., Besedovsky et al., FASEB 28:67-75 (2014)); prednisolone (as described in e.g., Wehling-Henricksa et al., Neuromuscular Disorders 14(8-9): 483-490 (2004); Raziuddin et al., Scandinavian Journal of Immunology, 31:139-145 (1990)); methylprednisolone (as described in e.g., Aristimuño et al., Journal of Neuroimmunology 204(1-2): 131-135 (2008); dexamethasone (as described in e.g., Hinrichs et al., J Immunother. 28(6):517-524 (2005)); metamethasone (as described in e.g., Kubin et al., Acta Derm Venereol 97:449-455 (2017)); triamcinolone (as described in e.g., pubchem.ncbi.nlm.nih.gov/compound/Triamcinolone); denosumab (Prolia®) (as described in e.g., Bekker PJ et al., J Bone Miner Res 19(7):1059-1066 (2004); Rossini M et al., Endocrine 53(3):857-859 (2016)); atacicept (as described in e.g., Dall'Era et al., Arthritis & Rheumatism 56(12):4142-4150 (2007); Xing et al., Oncotarget. 8(52): 89486-89499 (2017)); ocrelizumab (Ocrevus®) (as described in e.g., Laurent et al., ECTRIMS Online Library. 200348; P693; Oct. 26, 2017; Gingele et al., Cells 8(1),12 (2019)); obinutuzumab (Gazyvaro®) (as described in e.g., Garcia-Munoz et al., Immunotherapy. 10(6):491-499 (2018); bevacizumab (Blincyto®) (as described in e.g., Goeje et al., Clin Cancer Res 25(7):2219-2227 (2019); Elamin et al., Cancer Microenviron. 8(1):15-21 (2015); Li et al., Clin Cancer Res. 12(22):6808-6816 (2006)); or inotuzumab ozogamicin (Besponsa®) (as described in e.g., Carvello et al., Diabetes 61(1):155-165 (2012).

In some aspects, the pharmaceutical decreasing circulating levels of IgG is chosen from carbamazepine (as described in e.g., Ashrafi et al., Iran J Pediatr. 20(3):269-176 (2010); Mauri-Hellweg et al., J Immunol. 155(1):462-472 (1995); White et al., J Allergy Clin Immunol. 136(2):219-234 (2015)); sodium valproate (as described in e.g., Ashrafi et al., Iran J Pediatr. 20(3):269-176 (2010); Goodyear et al., Blood 116(11):1908-1918 (2010); Armeanu et al., Cancer Res 65(14):6321-6329 (2005); Poggi et al., Leukemia 23:641-648 (2009)); phenobarbital (as described in e.g., Ashrafi et al., Iran J Pediatr. 20(3):269-176 (2010); Hashizume et al., J Immunol. 168(10):5359-5368 (2002); Nishio et al., J Derm. Science 48(1):25-33 (2007)); phenytoin (Dilantin®) (as described in e.g., Agarwal et al., Ann Allergy Asthma Immunol. 99(3):281-283 (2012); Mauri-Hellweg et al., J Immunol. 155(1):462-472 (1995)); lenalidomide (Revlimid®) (as described in e.g., Shannon et al., Int lmmunopharmacol. 12(2):441-446(2012); Simone et al., Blood 122:119 (2013)); cloroquine (as described in e.g., Accapezzato et al., J Exp Med. 202(6):817-828 (2005); Garulli et al., Clinical and Vaccine Immunology 15(10):1497-1504 (2008)); amodiaquine (as described in e.g., Mak et al., Chem. Res. Toxicol. 28:1567-1573 (2015)); pyrimethamine (as described in e.g., Pierdominici et al., J Pharmacol Exp Ther. (3):1046-1057 (2005); F. Matthew Kuhlmann, James M. Fleckenstein, “Antiparasitic Agents,” Infectious Diseases (Fourth Edition) (2017)); proguanil (as described in e.g., Elliott et al., Infect Immun. 73(4): 2478-2485 (2005); Pombo et. al. Lancet.;360(9333):610-617(2002)); sulfonamides (as described in e.g., Mauri-Hellweg et al., J Immunol. 155(1):462-472(1995); Castrejon et al., Journal of Allergy and Clinical Immunology, 125(2):411-418.e4 (2010)); mefloquine (as described in e.g., Bijker et al., PLOS ONE 9(11): e112910 (2014); atovaquone (as described in e.g., Elliott et al., Infect Immun. 73(4): 2478-2485 (2005); Pombo et. al. Lancet.;360(9333):610-617(2002)); primaquine (as described in e.g., Berenzon et al., Journal of Immunology 171:2024-2034 (2003)); artemisinin (as described in e.g., Islamuddin et al., PLoS Negl Trop Dis. 9(1):e3321(2015)); doxycycline (as described in e.g., Kloppenburg et al., Clin Exp Immunot 102:635-641 (1995)); clindamycin (as described in e.g., Ekmekciu et al., Front Immunol. 8:397 (2017)); captopril (as described in e.g., Wysocki et al., Clin Cancer Res. (13):4095-4102 (2006); Silva Filho et al., Front.Cell. Infect. Microbiol. , 7(42):1-17 (2017)); cortisol (as described in e.g., Besedovsky et al., FASEB 28:67-75 (2014)); prednisone (as described in e.g., Salinas-Carmona M. C. et al., Autoimmunity. 42(6):537-544 (2009); Settipane, G. A. et al., J Allergy Clin Immunol. 62(3):162-166 (1978); Agarwal et al., Ann Allergy Asthma Immunol. 99(3):281-283 (2007); Giles et al., J ImmunoTherapy Cancer 6(51) (published Jun. 11, 2018)); prednisolone (as described in e.g., Wehling-Henricksa et al., Neuromuscular Disorders 14(8-9):483-490 (2004); Raziuddin et al., Scandinavian Journal of Immunology, 31:139-145 (1990)); methylprednisolone (as described in e.g., Aristimuño etal., Journal of Neuroimmunology 204(12): 131-135 (2008); dexamethasone (as described in e.g., Hinrichs et al., J Immunother. 28(6):517-524 (2005)); metamethasone (as described in e.g., Kubin et al., Acta Derm Venereol 97:449-455 (2017)); triamcinolone (as described in e.g., pubchem.ncbi.nlm.nih.gov/compound/Triamcinolone); fludrocortisone acetate (as described in e.g., Baeck et al., Allergy 64:978-994 (2009)); deoxycorticostetone acetate (as described in e.g., Mohammed-Ali et al., “Animal Models of Kidney Disease,” Animal Models for the Study of Human Disease (Second Edition) (2017); Perrotta et al., Cardiovascular Research, 114(3):456 467 (2018)); fenclofenac (as described in e.g., Cush et al., Arthritis & Rheumatism 33(5):623-633 (1990); Spiers et al., International Journal of Immunopharmacology 15(8):865-869(1993)); gold salts (as described in e.g., Hirohata et al., Clinical Immunology 91(2):226-233 (1999)); penicillamine (as described in e.g., Hirohata et al., Arthritis & Rheumatism 37(6):942-950 (1994); Hill et al., Journal of Neuroimmunology 97(1-2):146-153 (1999); Rosada et al., Clin Exp Rheumatol. 11(2): 143-148 (1993)); sulfasalazine (as described in e.g., Liptay et al., Br J Pharmacol. 137(5):608-620 (2002); Kang et al., Immunology. 98(1):98-103 (1999)).

### III. Antibodies

In certain aspects, the present disclosure encompasses use of an anti-CD16 antibody to block IgG-mediated activation of CD16⁺ T cells. Anti-human- CD16 antibodies (or VH and/or VL domains derived therefrom) suitable for use in the disclosure can be generated using methods well known in the art. Alternatively, art recognized anti-CD16 antibodies can be used. For example, purified mouse anti-human CD16, clone 3G8 antibody (BD Biosciences) can be used.

The 3G8 monoclonal antibody as used herein specifically binds to the 50-65 kDa transmembrane form of the IgG Fc Receptor (FcyRIII, also known as “CD16”), a human NK cell-associated antigen. CD16 is expressed on NK cells as well as macrophages and granulocytes. Reports indicate that CD16 plays a role in signal transduction and NK cell activation. The 3G8 antibody blocks the binding of soluble immune complexes to granulocytes. The 3G8 antibody is reported (in e.g., Vossebeld et al., Int J Biochem Cell Biol. 29(3):465-473 (1997)) to increase intracellular calcium levels in human neutrophils by interacting with both FcγRIIa and FcγRIIIb molecules. This antibody has also been reported to induce homotypic neutrophil aggregation.

In some aspects, mouse anti-human CD16, clone B73.1 antibody (BioLegend) (as described in e.g., Prodjinotho U, et al., PLoSNegl TropDis. 10.1371/journal.pntd.0005777. PubMed (2017)) can be used.

In certain aspects, mouse anti-human CD16, clone CB16 antibody (eBioscience) (as described in e.g., Yin et al., Scientific Reports 6, Article number: 26296 (2016); Kragstrup et al., Arthritis Res Ther. 16(1): R42 (2014)) can be used.

Anti-CD16 antibodies usable in the disclosed methods also include isolated antibodies that bind specifically to human CD16 and cross-compete for binding to human CD16 with any anti-CD16 antibody disclosed herein, e.g., the 3G8 antibody, the B73.1 antibody, or the CB16 antibody. In some aspects, the anti-CD16 antibody binds the same epitope as any of the anti-CD16 antibodies described herein, e.g., the 3G8 antibody, the B73.1 antibody, or the CB16 antibody. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., the 3G8 antibody, the B73.1 antibody, or the CB16 antibody by virtue of their binding to the same epitope region of CD16. Cross-competing antibodies can be readily identified based on their ability to cross-compete with e.g., the 3G8 antibody, the B73.1 antibody, or the CB16 antibody in standard CD16 binding assays such as Biacore analysis, ELISA assays or flow cytometry.

Techniques for determining whether two antibodies bind to the same epitope include, e.g., epitope mapping methods, such as, x-ray analyses of crystals of antigen:antibody complexes which provides atomic resolution of the epitope and hydrogen/deuterium exchange mass spectrometry (HDX-MS), methods monitoring the binding of the antibody to antigen fragments or mutated variations of the antigen, where loss of binding due to a modification of an amino acid residue within the antigen sequence is often considered an indication of an epitope component, computational combinatorial methods for epitope mapping.

In certain aspects, the antibodies that cross-compete for binding to human CD16 with, or bind to the same epitope region of human CD16 antibody as the 3G8 antibody, the B73.1 antibody, or the CB16 antibody are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-CD16 antibodies usable in the methods of the disclosure also include antigen-binding portions of the above antibodies. It has been amply demonstrated that the antigen-binding function of an antibody can be performed by fragments of a full-length antibody.

Anti-CD 16 antibodies suitable for use in the disclosed methods or compositions are antibodies that bind to CD16 with high specificity and affinity, block the binding of CD 16, and inhibit the immunosuppressive effect of the CD16 signaling pathway. In any of the compositions or methods disclosed herein, an anti-CD16 “antibody” includes an antigen-binding portion or fragment that binds to CD16 and exhibits the functional properties similar to those of whole antibodies in inhibiting receptor binding and up-regulating the immune system. In certain aspects, the anti-CD16 antibody or antigen-binding portion thereof cross-competes with the 3G8 antibody, the B73.1 antibody, or the CB16 antibody for binding to human CD16.

Anti-CD16 antibodies useful for the disclosure include antibodies engineered starting from antibodies having one or more of the V_{H} and/or V_{L} sequences disclosed herein, which engineered antibodies can have altered properties from the starting antibodies. An anti-CD16 antibody can be engineered by a variety of modifications for the engineering of modified anti-CD16 antibodies as known in the art.

### IV. Pharmaceutical Compositions

Pharmaceutical compositions suitable for administration to human patients are typically formulated for parenteral administration, e.g., in a liquid carrier, or suitable for reconstitution into liquid solution or suspension for intravenous administration.

In general, such compositions typically comprise a pharmaceutically acceptable carrier. As used herein, the term “pharmaceutically acceptable” means approved by a government regulatory agency or listed in the U.S. Pharmacopeia or another generally recognized pharmacopeia for use in animals, particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil, glycerol polyethylene glycol ricinoleate, and the like. Water or aqueous solution saline and aqueous dextrose and glycerol solutions can be employed as carriers, particularly for injectable solutions. Liquid compositions for parenteral administration can be formulated for administration by injection or continuous infusion. Routes of administration by injection or infusion include intravenous, intraperitoneal, intramuscular, intrathecal and subcutaneous. In one aspect, the mTOR inhibitor (e.g., temsirolimus) is administered intravenously.

### V. Patient Populations

Provided herein are methods of treating diseases or conditions which comprise a tumor, comprising the sequential steps of: (a) administering a dose regimen of an immunotherapy comprising dendritic cells loaded with RNA encoding a tumor antigen to a patient having a tumor; and (b) after administration of at least one dose of the immunotherapy of step (a), administering a dose regimen of a pharmaceutical which can cause one or more of the following: (i) decrease circulating IgG levels, (ii) block IgG-mediated activation of CD16+ T cells, (ii) decrease the concentration and/or function of B cells, (iv) reduce the frequency of CD38⁺ TGF-β⁺ B cells, (v) decrease B cell secretion of TGF-β, and (vi) sustain the frequency of CD25⁺CD28⁺ CD4 and/or CD8 T cells.

In some aspects of the disclosure, a tumor that can be treated using the methods of the disclosure can derived from, for example, renal cancer, breast cancer, pancreatic cancer, brain cancer (e.g., astrocytoma, glioblastoma multiforme), bone cancer, prostate cancer, colon cancer, lung cancer, cutaneous or intraocular malignant melanoma, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Waldenstrom macroglobulinaemia, Hodgkin’s Disease, non-Hodgkin’s lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia (AML), chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, pituitary adenoma, Kaposi’s sarcoma, epidermoid cancer, squamous cell cancer, T-cell lymphoma, environmentally induced cancers including those induced by asbestos, other B cell malignancies, and combinations of said cancers.

In some aspects of the disclosure, the tumor antigen is autologous to the patient. In some aspects, the patient’s tumor antigen can be newly expressed. In certain aspects, the patient’s tumor antigen can be a mutated normal protein antigen.

The present disclosure is also applicable to treatment of metastatic cancers (e.g., metastatic renal cell carcinoma (“mRCC”)).

In some aspects of the disclosure, the human patient suffers from mRCC. Upon diagnosis, the prognosis for patients with mRCC is classified into three overall disease risk profiles— favorable, intermediate, and poor—using objective prognostic risk factors. These risk factors were originally developed by the researchers at Memorial Sloan-Kettering Cancer Center (“MSKCC”) based on clinical data from patients treated with cytokine-based immunotherapies, such as interferon-α and IL-2, which were the standard of care for the treatment of mRCC prior to the approval of sunitinib and other newer agents in the past few years. The following are revised risk factors (“the Heng risk factors”), which have been correlated to overall survival in mRCC and include:
1. time from diagnosis to the initiation of systemic therapeutic treatment of less than one year, which is indicative of more aggressive disease (“less than one year to treatment risk factor”);
2. low levels of hemoglobin, a protein in the blood that carries oxygen;
3. elevated corrected calcium levels;
4. diminished patient performance status or physical functioning;
5. elevated levels of neutrophils, a type of white blood cell; and
6. elevated platelet count.

Patients exhibiting zero risk factors at the time of treatment are included in the favorable risk group; patients exhibiting one or two risk factors are included in the intermediate risk group; and patients exhibiting three or more risk factors are included in the poor risk group.

In some aspects of the disclosure, the patient is a poor risk human patient. In certain aspects, the poor risk patient exhibits three or more of the following risk factors: (i) time from diagnosis to the initiation of systemic therapeutic treatment of less than one year, (ii) low levels of hemoglobin, (iii) elevated corrected calcium levels, (iv) diminished patient performance status or physical functioning, (v) elevated levels of neutrophils, and (vi) elevated platelet count.

In some aspects, the tumor is the clear cell type. In certain aspects, the tumor is the non-clear cell type. The diagnosis of RCC is generally made by examination of a tumor biopsy under a microscope. Upon evaluation of the visual appearance of the tumor cells, the pathologist will classify the RCC into clear cell or non-clear cell types. According to National Comprehensive Cancer Network, approximately 85% of all RCC diagnoses are clear cell RCC.

### VI. Treatment Protocols

The target dose of CMN-001 is between about 6 to about 25 x 10⁶ DCs delivered by intradermal (i.d.) injections. The study population includes poor risk mRCC patients. The CMN-001 dosing can consist of the following three phases:
Induction phase: 3 doses, 3 weeks apart
Maintenance phase: 7 doses, 4 weeks apart; and
Booster phase: Every 12 weeks.

CMN-001 can be administered after or at the same time as dosing with a check point inhibitor, commonly referred to as an Immuno-Oncology (IO) agent. CMN-001 can be administered prior to, at the same time, or after the administration of an mTOR inhibitor (e.g., everolimus) and/or a TKI inhibitor (e.g., lenvatinib).

### VII. Outcomes

Patients treated according to the methods disclosed herein preferably experience improvement in at least one sign of cancer. In one aspect, improvement is measured by a reduction in the quantity and/or size of measurable tumor lesions. In another aspect, lesions can be measured on chest x-rays or CT or MRI films. In another aspect, cytology or histology can be used to evaluate responsiveness to a therapy.

In one aspect, the patient treated exhibits a complete response (CR), a partial response (PR), stable disease (SD), immune-related complete disease (irCR), immune-related partial response (irPR), or immune-related stable disease (irSD). In another aspect, the patient treated experiences tumor shrinkage and/or decrease in growth rate, i.e., suppression of tumor growth. In another aspect, unwanted cell proliferation is reduced or inhibited. In yet another aspect, one or more of the following can occur: the number of cancer cells can be reduced; tumor size can be reduced; cancer cell infiltration into peripheral organs can be inhibited, retarded, slowed, or stopped; tumor metastasis can be slowed or inhibited; tumor growth can be inhibited; recurrence of tumor can be prevented or delayed; one or more of the symptoms associated with cancer can be relieved to some extent.

In still other aspects, the methods of treatment produce a clinical benefit rate (CBR=CR+PR+SD≥6 months) better than that achieved by a method of treatment that does not comprise the steps of (a) administering a dose regimen of an immunotherapy comprising dendritic cells loaded with RNA encoding a tumor antigen to a patient having a tumor; and (b) after administration of at least one dose of the immunotherapy of step (a), administering a dose regimen of a pharmaceutical which can cause one or more of the following: (i) decrease circulating IgG levels, (iii) block IgG-mediated activation of CD16+ T cells, and (ii) decrease the concentration and/or function of B cells. In other aspects, the improvement of clinical benefit rate is about 20% 20%, 30%, 40%, 50%, 60%, 70%, 80% or more compared to a method of treatment that does not comprise the steps of (a) administering a dose regimen of an immunotherapy comprising dendritic cells loaded with RNA encoding a tumor antigen to a patient having a tumor; and (b) after administration of at least one dose of the immunotherapy of step (a), administering a dose regimen of a pharmaceutical which can cause one or more of the following: (i) decrease circulating IgG levels, (ii) block IgG-mediated activation of CD16+ T cells, (iii) decrease the concentration and/or function of B cells, (iv) reduce the frequency of CD38⁺ TGF-β⁺ B cells, (v) decrease B cell secretion of TGF-β, and (vi) sustain the frequency of CD25⁺CD28⁺ CD4 and/or CD8 T cells.

In still other aspects, the methods of treatment produce an objective response rate (ORR=CR+PR) of at least about 15%, at least about 20%, at least about 25%, at least about 30%, at least about 40%, at least about 50%, at least about 60%, at least about 70%, at least about 80%, at least about 90%, or about 100%. In some aspects, the median duration of response is ≥ 3 month, > 6 month, > 12 month, or ≥ 18 month. In one aspect, the median duration of response is ≥ 6 month. In some aspects, the frequency of patients with duration of response ≥ 6 month is at least about 30%, at least about 40%, at least about 50%, at least about 60%, at least about 70%, at least about 80%, at least about 90%, at least about 95%, at least about 99% or 100%.

In still other aspects, the methods of treatment produce an objective response rate (ORR=CR+PR) better than that achieved by a method of treatment that does not comprise the steps of (a) administering a dose regimen of an immunotherapy comprising dendritic cells loaded with RNA encoding a tumor antigen to a patient having a tumor; and (b) after administration of at least one dose of the immunotherapy of step (a), administering a dose regimen of a pharmaceutical which can cause one or more of the following: (i) decrease circulating IgG levels, (ii) block IgG-mediated activation of CD16+ T cells, (iii) decrease the concentration and/or function of B cells, (iv) reduce the frequency of CD38⁺ TGF-β⁺ B cells, (v) decrease B cell secretion of TGF-β, and (vi) sustain the frequency of CD25⁺CD28⁺ CD4 and/or CD8 T cells. In other aspects, the improvement of objective response rate is about 20% 20%, 30%, 40%, 50%, 60%, 70%, 80% or more compared to a method of treatment that does not comprise the steps of (a) administering a dose regimen of an immunotherapy comprising dendritic cells loaded with RNA encoding a tumor antigen to a patient having a tumor; and (b) after administration of at least one dose of the immunotherapy of step (a), administering a dose regimen of a pharmaceutical which can cause one or more of the following: (i) decrease circulating IgG levels, (ii) block IgG-mediated activation of CD16+ T cells, and (iii) decrease the concentration and/or function of B cells, (iv) reduce the frequency of CD38⁺ TGF-β⁺ B cells, (v) decrease B cell secretion of TGF-β, and (vi) sustain the frequency of CD25⁺CD28⁺ CD4 and/or CD8 T cells. In some aspects, the median duration of response is ≥ 3 month, ≥ 6 month, ≥ 12 month, or ≥ 18 month. In one aspect, the median duration of response is ≥ 6 month.

In still other aspects, the methods of treatment produce a disease control rate (DRR=CR+PR+SD) of at least about 20%, at least about 30%, at least about 40%, at least about 50%, at least about 60%, at least about 70%, at least about 80%, at least about 90%, at least about 95%, at least about 99% or about 100%. In one aspect, the methods of treatment produce a disease control rate of at least about 70%, wherein the malignant tumor is a LAG-3 positive melanoma that is resistant to treatment with an anti-PD1 or anti-PD-L1 antibody. In some aspects, the median duration of response is ≥ 3 month, ≥ 6 month, ≥ 12 month, or ≥ 18 month. In one aspect, the median duration of response is ≥ 6 month. In some aspects, the frequency of patients with duration of response ≥ 6 month is at least about 30%, at least about 40%, at least about 50%, at least about 60%, at least about 70%, at least about 80%, at least about 90%, at least about 95%, at least about 99% or 100%.

In still other aspects, the methods of treatment produce a disease control rate (DRR=CR+PR+SD) better than that achieved by a method of treatment that does not comprise the steps of (a) administering a dose regimen of an immunotherapy comprising dendritic cells loaded with RNA encoding a tumor antigen to a patient having a tumor; and (b) after administration of at least one dose of the immunotherapy of step (a), administering a dose regimen of a pharmaceutical which can cause one or more of the following: (i) decrease circulating IgG levels, (ii) block IgG-mediated activation of CD16⁺ T cells, (iii) decrease the concentration and/or function of B cells , (iv) reduce the frequency of CD38⁺ TGF-β⁺ B cells, (v) decrease B cell secretion of TGF-β, and (vi) sustain the frequency of CD25⁺CD28⁺ CD4 and/or CD8 T cells. In other aspects, the improvement of disease control rate is about 20% 20%, 30%, 40%, 50%, 60%, 70%, 80% or more compared to a method of treatment that does not comprise a step of (i) determining the level of LAG-3 expression in a tumor sample prior to treatment, (ii) selecting a LAG-3 positive tumor for treatment, (iii) treating a tumor that has been identified as LAG-3 positive prior to treatment, or (iv) any combinations thereof. In some aspects, the median duration of response is ≥ 3 month, ≥ 6 month, ≥ 12 month, or ≥ 18 month. In one aspect, the median duration of response is ≥ 6 month.

### VIII. Kits and Unit Dosage Forms

Also within the scope of the present disclosure are kits for treating a patient afflicted with disease or condition which comprise a tumor (e.g., renal cell cancer). Kits typically include a label indicating the intended use of the contents of the kit and instructions for use. The term “label” includes any writing, or recorded material supplied on or with the kit, or which otherwise accompanies the kit.

In one aspect, the kit, for example, comprises:
(a) a dose of an immunotherapy comprising dendritic cells loaded with RNA encoding a tumor antigen to a patient having a tumor;
(b) a dose of a pharmaceutical which can cause one or more of the following: (i) decrease circulating IgG levels, (ii) block IgG-mediated activation of CD16+ T cells, (iii) decrease the concentration and/or function of B cells, (iv) reduce the frequency of CD38⁺ TGF-β⁺ B cells, (v) decrease B cell secretion of TGF-β, and (vi) sustain the frequency of CD25⁺CD28⁺ CD4 and/or CD8 T cells; and
(c) instructions for using an immunotherapy and the pharmaceutical in the methods described herein.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); ); Crooks, Antisense drug Technology: Principles, strategies and applications, 2^{nd} Ed. CRC Press (2007) and in Ausubel et al. (1989) Current Protocols in Molecular Biology (John Wiley and Sons, Baltimore, Md.).

The following examples are offered by way of illustration and not by way of limitation.

### EXAMPLES

The following experimental methods and details are referenced in the Examples that follow.

### Example 1

### Materials and Methods

Unless provided otherwise, the Examples described below use one or more of the following materials and methods:

### Blood Sample Collection and Processing

Human subjects enrolled on the ADAPT trial (NCT01582672) provided written informed consent prior to participation and the trail was approved by each institutional review board in accordance with the Declaration of Helsinki and Good Clinical Practice Guidelines of the International Conference on Harmonization. Peripheral blood samples were collected in sodium heparin tubes (Becton-Dickinson, NJ, USA) from consenting mRCC patients or by a leukapheresis collection from healthy volunteers provided by Key Biologics (Memphis, TN). Separation of plasma from blood was performed by centrifugation while peripheral blood mononuclear cells (PBMCs) were isolated using Histopaque-1077 (Sigma-Aldrich). Separated plasma was aliquoted and frozen at -80° C. while PBMCs were resuspended in FBS (Atlanta Biologicals) containing 10% DMSO (Sigma-Aldrich), and stored in liquid nitrogen. Plasma was thawed at room temperature and heated to 56° C. for 30 minutes, briefly centrifuged to remove precipitated protein and used immediately or re-frozen at -80° C.

### Cell Culture

PBMCs and DCs were thawed and washed in PBS (Lonza) prior to counting. Post maturation electroporated monocyte-derived dendritic cells (PME-CD40L) were generated from healthy donors as described in DeBenedette MA. et al., J Immunol. 181(8):5296-5305 (2008) (incorporated herein by reference in its entirety), and were co-electroporated with RNA encoding the pp65 CMV protein and RNA encoding CD40L. Viable cell counts were performed using BD TruCount Absolute Counting Tubes and propidium iodide (BD Biosciences) by flow cytometry. PBMCs and DCs were re-suspended in X-Vivo15 (with gentamicin and phenol red) (Lonza) containing 10% normal human AB serum (Valley biomedical). One million PBMCs were mixed with DCs at a 10:1 PBMC:DC ratio in sterile Falcon 5 mL polypropylene round bottom test tubes (Corning Life Sciences) along with healthy donor or mRCC patient plasma with or without the addition of everolimus (LC Laboratories) and incubated in a 5% CO₂ incubator at 37° C. for six days. Frozen PBMC vials from 2 HLA-mismatched healthy donors were thawed and counted. One million viable PBMCs from each donor were added to sterile Falcon 5 mL polypropylene round bottom test tubes along with healthy donor or mRCC patient plasma with or without the addition of everolimus and incubated in a 5% CO₂ incubator at 37° C. Purified Mouse anti-human CD3 (clone OKT3, BD Biosciences) was used to stimulate healthy donor PBMCs in culture. 0.5 µg of OKT3 was incubated with one million viable PBMCs with or without the addition of mRCC patient plasma and incubated in a 5% CO₂ incubator at 37° C. for seven days.

### Cell Surface and Intracellular Staining

T cells were identified using APC-H7 Mouse Anti-Human CD3 antibody (clone SK7, BD Biosciences). T cell subsets were identified using Pacific Blue Mouse Anti-Human CD4 antibody (clone RPA-T4, BD Biosciences) and PerCP-Cy5.5 Mouse Anti-Human CD8 antibody (clone SK1, BD Biosciences). Activated T cells expressing the IL-2 alpha receptor were identified using Brilliant Violet 605 Mouse Anti-Human CD25 antibody (clone 2A3, BD Biosciences). The T cell costimulatory receptor that binds CD80 and CD86 was identified using PE Mouse Anti-Human CD28 antibody (clone CD28.2, BD Biosciences). B cells were identified using APC-eFluor 780 Mouse Anti-Human CD19 antibody (clone HIB19, ThermoFisher Scientific). Terminally differentiated B cells were identified using APC Mouse Anti-Human CD38 antibody. Proliferating cells were identified using Brilliant Violet Mouse Anti-Ki-67 antibody (clone B56, BD Biosciences). The heavy chain of immunoglobulin G subclass was identified using PE-CF594 Mouse Anti-Human IgG antibody (clone G18-145, BD Biosciences). The multifunctional cytokine Human Transforming Growth Factor beta-1 was identified using PerCP-Cy5.5 Mouse anti-Human LAP (TGF-β1) antibody (clone TW4-2F8, Biolegend). 5 mL tubes containing cultured cells were centrifuged at 400 g for 5 minutes after which supernatant was decanted. Pelleted cells were washed by adding 2 mL of Stain Buffer containing fetal bovine serum and ≤0.09% sodium azide (BD Biosciences), briefly vortexed and centrifuged. Stain Buffer was decanted from tubes and pelleted samples were then surfaced stained with monoclonal antibody, briefly vortexed and incubated at room temperature for 15 minutes in the dark. Samples were then washed with 2 mL of PBS, centrifuged and resuspended in 1 mL of PBS. 2 µL of LIVE/DEAD Fixable Aqua Dead Cell dye (ThermoFisher Scientific) was added to each sample and incubated at 37° C. for 15 minutes. Samples were washed and centrifuged twice with 2 mL of Stain Buffer. Decanted and pelleted cells were vortexed and then fixed with 1 mL of diluted Transcription Factor Fix/Perm Buffer (BD Biosciences) for 15 minutes at room temperature. 1 mL of 1x Transcription Factor Perm/Wash Buffer (BD Biosciences) was added to each sample, briefly vortexed and centrifuged. Perm/Wash buffer was decanted from samples and washed again with 2 mL of Perm/Wash buffer. Perm/Wash Buffer was decanted from tubes and pelleted samples were then stained for intracellular antigens with monoclonal antibody, briefly vortexed and incubated at room temperature for 15 minutes in the dark. After incubation, samples were washed twice with Perm/Wash Buffer and resuspended in 350 µL of Stain Buffer.

### Flow Cytometry Acquisition and Analysis

Samples were acquired using a special order BD LSRII flow cytometer configured with blue (488 nm), green (532 nm), red (633 nm), and violet (405 nm) lasers. UltraComp eBeads (ThermoFisher Scientific) were used with individual fluorochrome-conjugated antibodies for use as single-color compensation controls. BD FACSDiva software (BD Biosciences) was used for sample acquisition. Sample analysis was performed using FlowJo software v9.9.6 (Tree Star, Inc.).

### B Cell Stimulation

Frozen patient PBMC were thawed, counted, and washed in PBS. The cells were resuspended in X-VIVO 15 media (with gentamicin and phenol red; Lonza) at 2x10⁶ cells/mL and incubated overnight in a 5% CO₂ incubator at 37° C. The cells were counted and set up in culture at 1x10⁶ cells/mL in X-VIVO 15 media. Cells were stimulated with a combination of CPG oligodeoxynucleotide (ODN) 2006 (2.5 µg/mL; InvivoGen), anti-human IgM (25 µg/mL; Jackson Labs), F(ab')2 anti-human IgG, IgM (H⁺L) (25 µg/mL; Invitrogen), LEAF purified anti-human CD40 (clone HB14, 1.0 µg/mL; Biolegend), IL-2 (120 UI/mL; ProLeukin), IL-4 (4 ng/mL; R&D Systems), and IL-21 (10 ng/mL; R&D Systems) See e.g., Dedobbeleer O. et al., J Immunol., 199(2):391-396 (2017), incorporated herein by reference in its entirety. Everolimus (LC Laboratories) was added at 10 ng/mL to unstimulated or stimulated cultures. The cultures were incubated overnight in a 5% CO₂ incubator at 37° C. for six days.

### Detection of TGF-β1

The concentration of total TGF-β1 in plasma and supernatant samples was quantified using the LEGEND MAX Total TGF-β1 ELISA Kit (Biolegend). Samples were prepared by acid treatment to activate latent TGF-β1 and then diluted 1:20 or 1:100 in assay buffer. Samples were incubated on a plate pre-coated with anti-TGF-β1 antibody for 2 hours. Anti-TGF-β1 was incubated for 1 hour, and the plate was washed and incubated with Avidin-HRP for 30 minutes. The plate was developed with TMB substrate solution and Stop solution, and was read at 450 nm and 570 nm using an ELx800 plate reader.

### Statistics

Statistical analysis was performed on Microsoft Excel 2016 software version (Santa Cruz, CA) variances of mean values are presented as bar and whisker plots. Statistical comparisons between groups were calculated using a paired τ-test. p values ≤ 0.05 were considered statistically significant.

### Example 2

### Generation of Antigen Expressing Dendritic Cells by Electroporation

300 x 10⁶ peripheral blood mononuclear cells (PBMCs) were cultured in 30 mL AIM-V media (Thermo Fisher Scientific) in T150 flasks (Corning) for 2 hours to provide for the adherence of monocytes. Non-adherent cells were removed by washing the monolayers twice with cold phosphate buffered saline (PBS) (Cambrex), and the remaining monocytes were cultured in X-VIVO 15 medium (Cambrex) for 5 days, supplemented with 1000 U/mL each of granulocyte macrophage-colony stimulating factor (GM-CSF) (Bayer AG, Leukine® liquid) and IL-4 (R&D Systems). To achieve DC maturation, immature DCs were first cultured on day 5 with 10 ng/mL tumor necrosis factor alpha (TNF-α) (R&D Systems), 1000 µ/mL interferon gamma (IFN-y) (Actimmune), and 1 µg/mL prostaglandin E2 (PGE2) (Sigma). On day 6, phenotypically mature DCs were co-electroporated with RNA encoding CD40L (Argos Lot No. 101606 JSC) at a concentration of 3 ug per million DCs plus either human cytomegalovirus (CMV) pp65 or MART-1 encoding RNAs at 2 µg per million DCs (CMVpp65 RNA-Argos Lot No 071107XF, MART-1-APL RNA-Argos Lot No. 011909HC). Generation of MART-1 RNA and CD40L RNA is described in e.g., DeBenedette et al., J. Immunol. 181(8):5296-5305 (2008).

Electroporated DCs were cultured for an additional 4 hours in X-VIVO 15 media supplemented with 800 µ/mL GM-CSF and 500 µ/mL IL-4 at 1 x 10⁶ DCs/mL in low adherence 6-well plates (Costar). After 4 hr, the DCs were harvested by washing with cold PBS and prepared as a frozen formulation at 10 x 10⁶ DCs/mL in 20% DMSO and 80% FBS, representing antigen electroporated Post Maturation Electroporated (PME)-CD40L DC preparations.

### Example 3

### Generation of MART-1 and CMV pp65 Specific CTL

PME-CD40L DCs derived from HLA-A2-positive donors and transfected with antigen-encoding mRNAs were co-cultured with purified CD8⁺ T cells. All co-cultures were performed in R-10 media. CD8⁺ T cells were purified using the CD8+ T Cell Negative Selection Kit II (Miltenyi Biotec) from non-adherent cells harvested from the monocyte adherence step. The CD8⁺ cells were mixed with DCs at a 10:1 ratio and incubated in the presence of 0.2 µg/mL IL-2 and 10 ng/mL IL-7 (R&D Systems). DC/T cell co-cultures were re-stimulated on day 7 and supplemented with 20 units/mL IL-2 plus 10 ng/mL IL-7. Induction of primary immune responses to MART-1 and memory/recall responses to CMV pp65 were measured on day 10.

### Example 4

### Induction of CD8⁺ T Cell Immune Responses to Cytomegalovirus Antigen pp65 and To Melanoma Associated Antigen MART-1

### Analysis of T Cell Specificity and Function

To identify antigen-specific T cell responses and the phenotype of the antigen specific cells, T cells were harvested on day 10 post-stimulation and incubated with peptide-pulsed T2 target cells at a ratio of 10:1 for 4 hour with Brefeldin A, Monensin (BD Biosciences) and CD107a PE-Cy5. T2 Cells were peptide pulsed with HLA-A2 restricted epitopes for MART-APL (LAGIGILTV), CMV pp65 (NLVPMVATV) or control peptide from prostate specific antigen (PSA-FLTPKKLQCV) by incubation for 1.5 hrs in R-10 media. After 4 hours of co-culture, responder T cells were stained for 10 minutes with the requisite peptide/pentamer reagent, then washed once and re-stained with CD8-Eflour605, CD45RA-FITC, and CD28-APC. Cells were then washed in PBS and stained with Aquadye (BD Biosciences) for analysis of viability. Labeled cells were then fixed and permeabilized in Fixation/Permeabilization solution (eBiosciences). Cells where then stained with IFN-γ-PECy7, TNFa-AF700 and IL-2-PerCPcy5.5 in permeabilization buffer and resuspended in FACs buffer prior to acquisition.

### Induction of CD8⁺ T Cell Immune Responses to Cytomegalovirus Antigen pp65

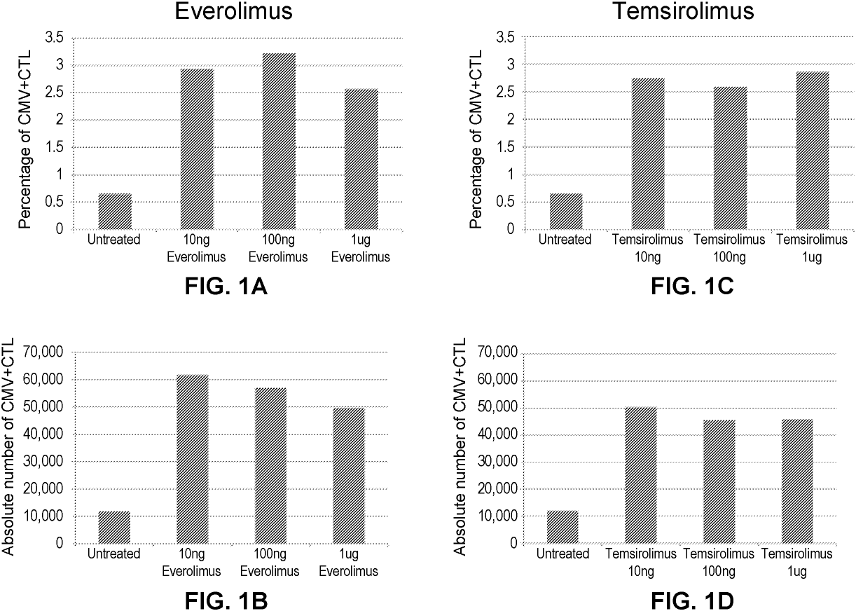

CD8⁺ cytotoxic T lymphocyte (CTL) responses were used to measure the pp65 CMV recall response induced by PME-CD40L DCs in the presence of mTOR inhibitors everolimus and temsirolimus. PME-CD40L DCs encoding CMV pp65 were used to stimulate recall CTL responses in the presence of the mTOR inhibitors under evaluation for compatibility with the CMN-001 product. The expansion of recall CTLs specific for CMV pp65 was determined on day 3 after a second weekly stimulation with PME-CD40L DCs. The percentage of cells within a defined gate (FIG. 1A and FIG. 1C) and absolute number (FIG. 1B and FIG. 1D) of CMV pp65 specific CTLs were determined using CMV pp65 peptide loaded MHC pentamer molecules in the presence of varying concentrations of everolimus or temsirolimus. Compared to DC/CTL co-cultures set up in the absence of the mTOR inhibitors, there was an increase in both the percentage and numbers of CMV pp65 specific CTLs in the presence of both mTORs at the lowest concentration tested (10 ng/ml). This increase in the percentage and absolute numbers of CMV⁺ CTL was evident even at the higher concentrations of mTORs tested (1 µg/ml).

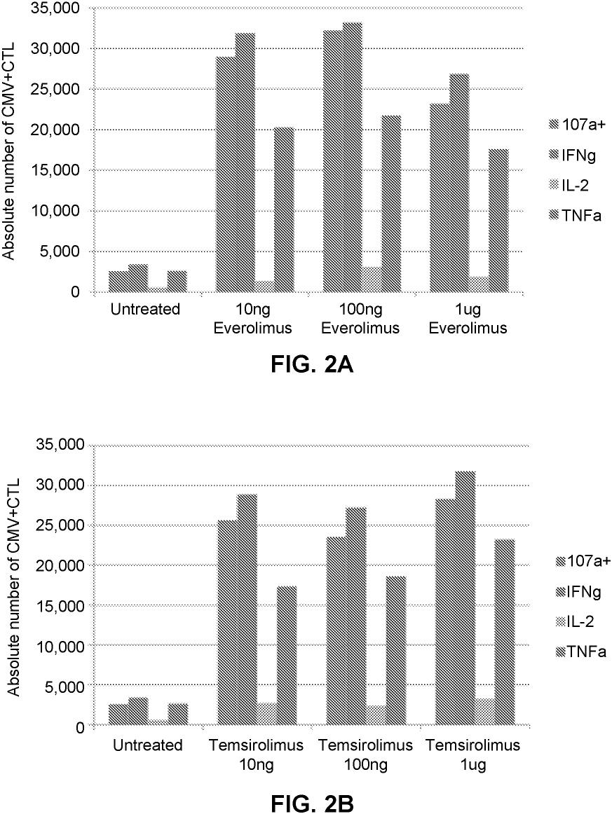

To assess the impact mTOR inhibition would have on CTL effector function in vitro, CMV pp65 specific CTLs were assayed for the ability to secret the cytokines IFN-y, TNF-α and IL-2, and express the degranulation marker CD107a, which serves as a surrogate marker to measure lytic activity. Data presented in FIG. 2A and FIG. 2B measures the absolute numbers of CMV pp56 specific CTLs that secrete IFN-y, TNF-α or IL-2, or express CD107a in response to CMV pp65 peptide pulsed target cells. The absolute numbers of CMV⁺ CTLs shown have been corrected for background non-specific responses to display only the antigen specific response for each parameter. The numbers of CMV⁺ CTL secreting a cytokine or expressing CD107a was increased in the presence of both mTOR inhibitors tested compared to untreated CTLs. Furthermore this increase in effector function was seen at the lowest concentration tested (10 ng/ml) and was maintained even at the highest concentration tested (1 µg/ml) for both everolimus (FIG. 2A) and temsirolimus (FIG. 2B).

### Induction of Primary CD8⁺ T Cell Immune Responses to the Melanoma-Associated Antigen MART-1

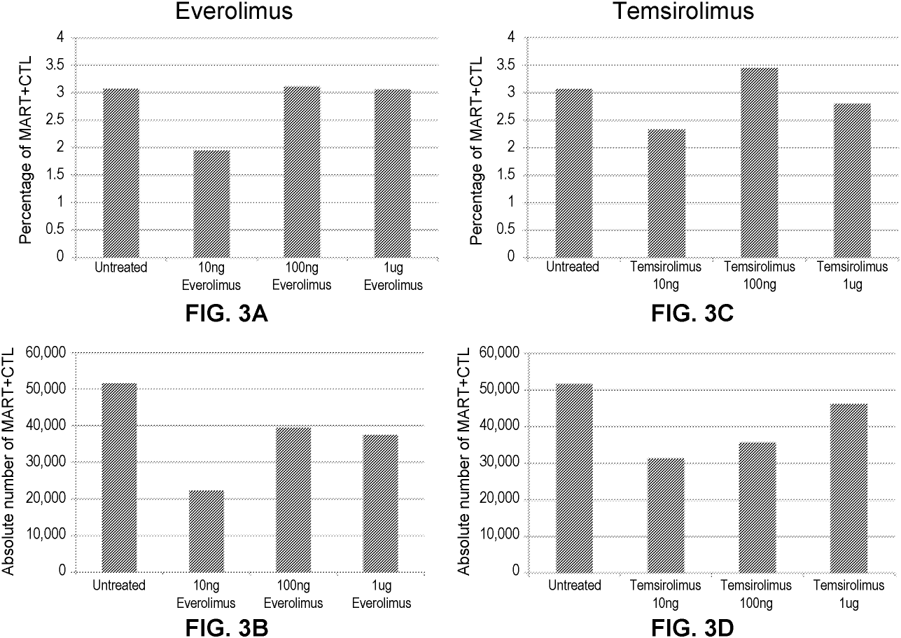

A similar analysis of MART-1 specific CTLs priming in the presence of mTOR inhibitors was undertaken. PME-CD40L DCs encoding MART-1 antigen were used to prime CTL responses in the presence of the mTOR inhibitors everolimus and temsirolimus over the concentrations indicated in FIGS. 3A-3D. The expansion of primed CTLs specific for MART-1 was determined on day 3 after a second weekly stimulation with PME-CD40L DC. The percentage (FIG. 3A and FIG. 3C) and absolute numbers (FIG. 3B and FIG. 3D) of MART-1 specific CTLs were determined using MART-1 peptide loaded MHC pentamer molecules. PME-CD40L DC encoding the MART-1 antigen where able to prime MART-1 specific CTLs in the presence of both everolimus and temsirolimus. Similar levels of percentages and absolute numbers of MART-1⁺ CTL were induced in the presence of everolimus (FIG. 3A and FIG. 3B) and temsirolimus (FIG. 3C and FIG. 3D).

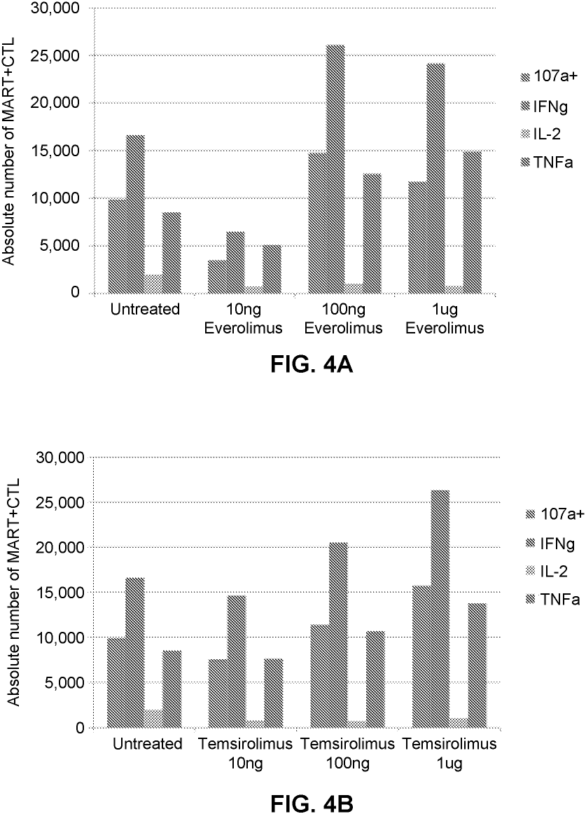

To assess the impact mTOR inhibitor treatment would have on CTL effector function in vitro, MART-1 specific CTL were assayed for the ability to secret the cytokines IFN-y, TNF-α and IL-2, and express the degranulation marker CD107a which serves as a surrogate marker to measure lytic activity. Data presented in FIG. 4A and FIG. 4B measures the absolute numbers of MART-1 specific CTLs that secrete IFN-γ, TNF-α or IL-2, or express CD107a in response to MART-1 peptide pulsed target cells. The absolute numbers of MART-1⁺ CTLs shown have been corrected for background non-specific responses to display only the antigen specific response for each parameter. The numbers of MART-1⁺ CTL secreting a cytokine or expressing CD107a was increased in the presence of both everolimus (FIG. 4A) and temsirolimus (FIG. 4B) at the higher concentrations tested compared to untreated CTLs.

### Example 5

### Inhibition of B Cell Secretion of IgG

### Cellular Antigen Staining by Multi-Color Flow Cytometry

Cultured cells were centrifuged and surface stained in 100 µl for 15 minutes at room temperature with conjugated antibody CD3 (UCHT1) APC-eFluor 780, CD16 (ebioCB16), CD56 APC (TULY56) (eBioscience), CD4 (RPA-T4) Pacific Blue, CD8 (SK1) PerCP-Cy5.5, Granzyme b (GB11) FITC, CD279 (EH12.1) PE-Cy7, CD25 (2A3) BV605, CD184 (12G5) PE-Cy5 (BD Biosciences), CD45RA (HI100) BV570 (Biolegend), CD14 (TUK4) Qdot 655 (Life Technologies) and CMV pp65 MHC dextramer or pentamer PE (Immudex or ProImmune respectively). Cells were washed twice with PBS (Cambrex) and stained with Live/Dead Fixable Aqua fluorescent reactive dye (Invitrogen) for 15 minutes at 37° C. in 1ml PBS. Cells were washed twice with Stain Buffer (FBS) (BD Bioscience) and fixed with Transcription Factor Buffer Set (BD Bioscience) for 15 minutes at room temperature. Cells were washed twice with Perm Wash Buffer and stained in 100µ1 for 15 minutes at room temperature with conjugated antibody IgG (G18-145), PE-CF594 (BD Biosciences), FoxP3 PE (206D) (Biolegend) and Ki67 BV711 (Biolegend). Cells were washed twice with Perm Wash Buffer, re-suspended in 350µ1 of Stain Buffer (FBS) and transferred to Trucount Absolute Counting Tubes (BD Biosciences). Samples were acquired using a BD LSRII Special Order System (BD Biosciences). Analysis was performed using Flowjo software ver.9.9.4 (Tree Star, Inc.)

### Proliferation of Peripheral Blood Lymphocytes from mRCC Patient

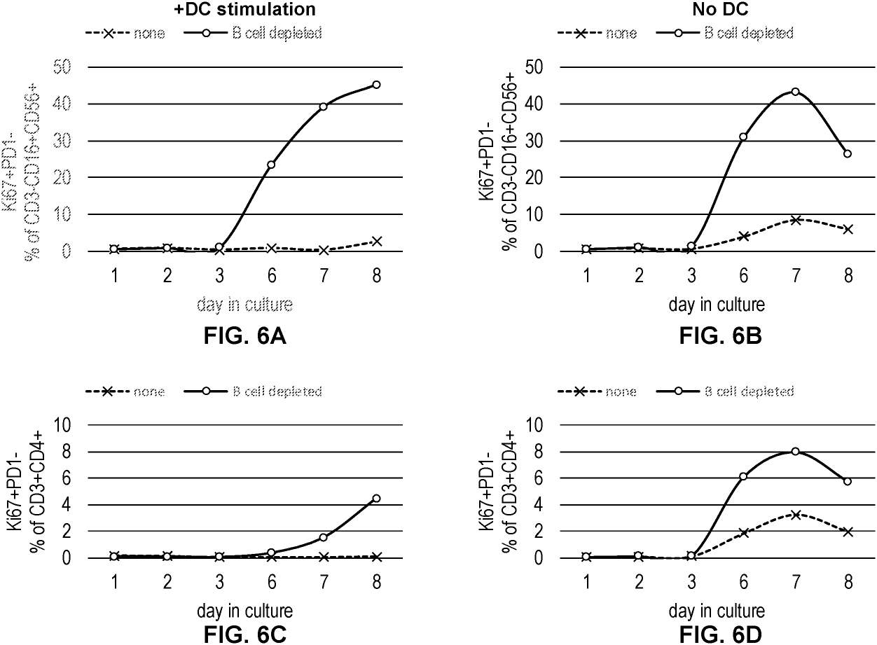

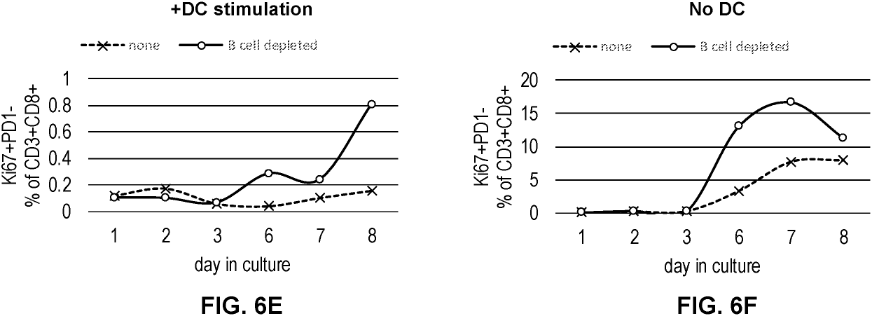

Mononuclear cells from an mRCC patient were isolated using Histopaque-1077 (Sigma-Aldrich), re-suspended in FBS (Atlanta Biologicals) containing 10% DMSO (Sigma-Aldrich) and stored in liquid nitrogen. Thawed PBMCs were re-suspended in 20 ml of X-Vivo 15 (Lonza). Anti-human CD19 microbeads (Miltenyi Biotec) were used to deplete CD19⁺ B cells from thawed PBMC according to the manufacturer’s instructions. Briefly, PBMC were magnetically labeled with CD19 microbeads and loaded onto a LD Macs Column. The unlabeled cell fraction was collected while the CD19⁺ B cells remained in the Macs Separator magnetic field. PBMC with or without B cell depletion were re-suspended in X-Vivo 15 containing 5% heat inactivated human AB serum (Valley Biomedical) at a concentration of 1 x 10⁶ cells/ml and incubated for 8 days with or without autologous generated PME-CD40L DCs electroporated with RNA encoding total tumor protein at 37° C. ± 1° C., 5% CO2 ± 1%, 65-95% relative humidity. B cell depleted lymphocytes demonstrate increased proliferation determined by increased detection of Ki67 at day 6 through day 8 over lymphocytes from PBMC not depleted of B cells with peak expression at day 7 when stimulated with DC. CD3⁻CD16⁺CD56⁺NK cells from B cell depleted PBMCs are 45.1% Ki67⁺PD1⁻ at day 8 without DCs stimulation, compared to CD3⁻CD16⁺CD56⁺ NK cells from PBMCs (cultured without B cell depletion and DCs stimulation) are 2.7% Ki67⁺PD1⁻ (FIG. 6A). B cell depleted PBMC continue to show higher NK cell proliferation with DCs stimulation (26.3% versus 6.0%) at day 8 (FIG. 6B). CD4⁺T cells (CD3⁺CD4⁺) from B cell depleted PBMCs are 4.4% Ki67⁺PD1⁻ at day 8 without DCs stimulation, while CD4⁺T cells from non-B cell depleted PBMCs are 0.1% Ki67+PD1- at day 8 without DCs stimulation (FIG. 6C). B cell depleted PBMC continue to show higher CD4⁺ T cell proliferation with DCs stimulation (5.7% versus 2.0%) at day 8 (FIG. 6D). CD8⁺ T cells (CD3⁺CD8⁺) from B cell depleted PBMCs are 0.8% Ki67⁺PD1⁻ at day 8 without DCs stimulation, while CD8⁺T cells from non-B cell depleted PBMCs are 0.2% Ki67⁺PD1⁻ at day 8 without DCs stimulation (FIG. 6E). B cell depleted PBMCs continue to show higher CD8⁺ T cell proliferation with DCs stimulation (11.3% versus 7.9%) at day 8 (FIG. 6F).

### PME-CD40L DCs from mRCC Patient Induce FoxP3⁺/CD25⁺/PD1⁺Expression in CD4^{hi} T Cells

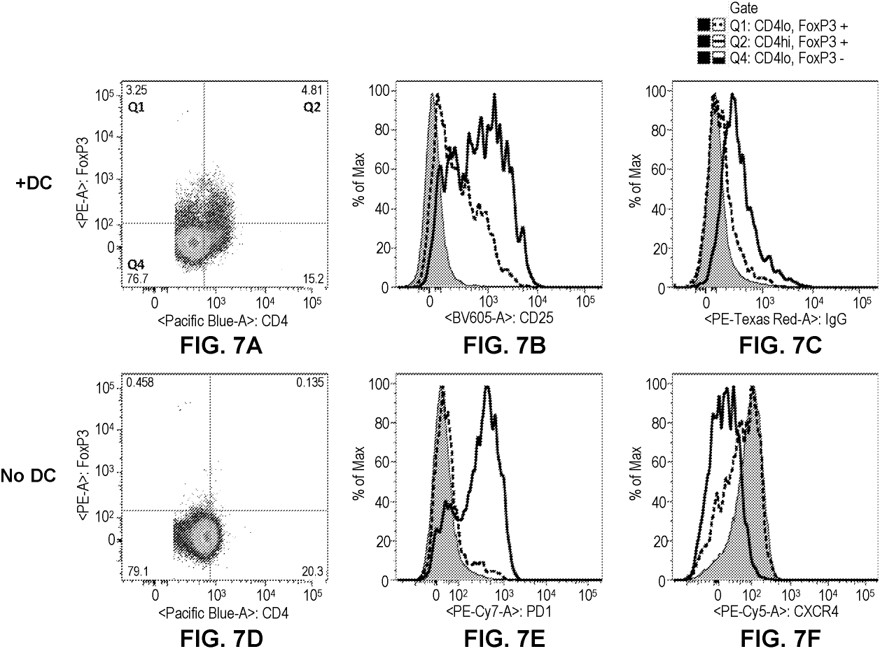

Day 7 cultured PBMC from mRCC patient reveals FoxP3 low expression without DCs stimulation (FIG. 7D), but higher when stimulated with PME-CD40L DCs (FIG. 7A) at day 7 when gating CD3⁺CD4⁺ cells. CD3⁺CD4^{hi,} FoxP3⁺ cells (solid line in histogram) show increased expression of CD25 (FIG. 7B), intracellular IgG (FIG. 7C), and PD1 (FIG. 7E) relative to CD3⁺CD4^{low}, FoxP3⁺ cells (dashed line in histogram) and CD3⁺CD4^{low}, FoxP3⁻ cells (shaded histogram). CD3⁺CD4^{hi}, FoxP3⁺ cells (solid line in histogram) show lower expression for the chemokine receptor CXCR4 relative to CD3⁺CD4^{low}, FoxP3⁺ cells (dashed line in histogram) and CD3⁺CD4^{low}, FoxP3⁻ cells (shaded histogram) (FIG. 7F).

### Anti-CD16 Antibody Blocks Intracellular IgG Uptake in CD4^{high} Expressing Lymphocytes Induced by PME-CD40L DCs from mRCC Patient

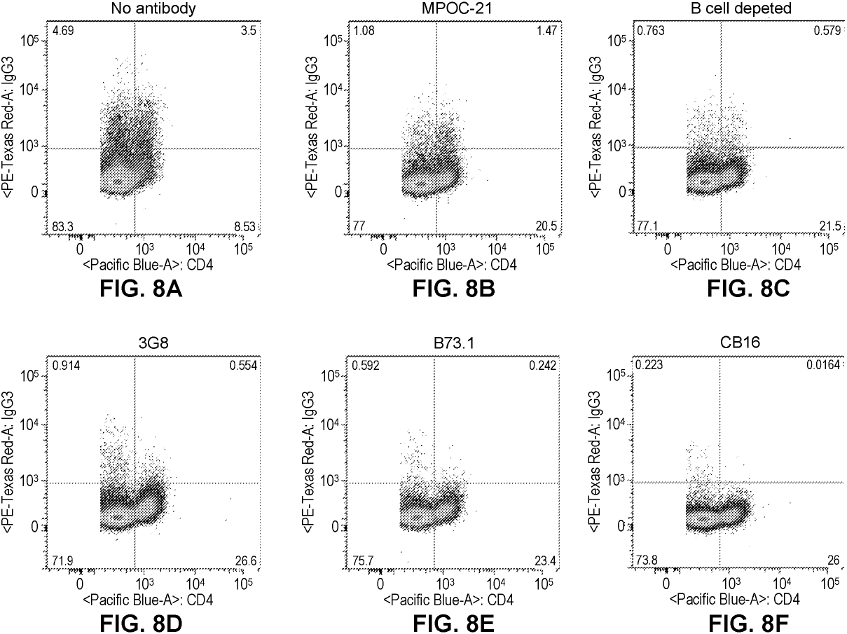

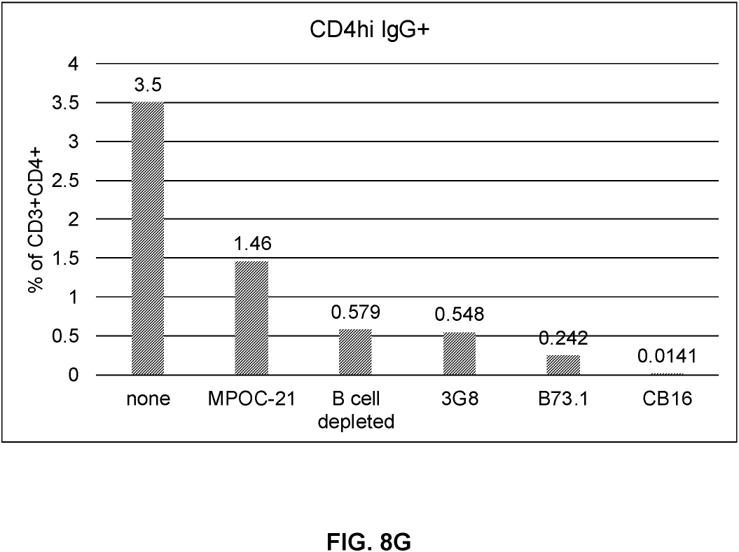

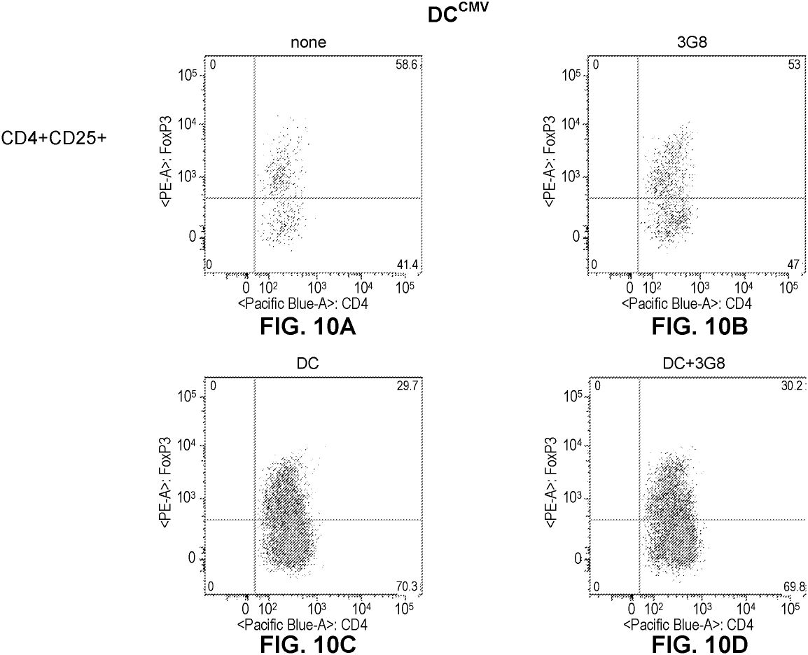

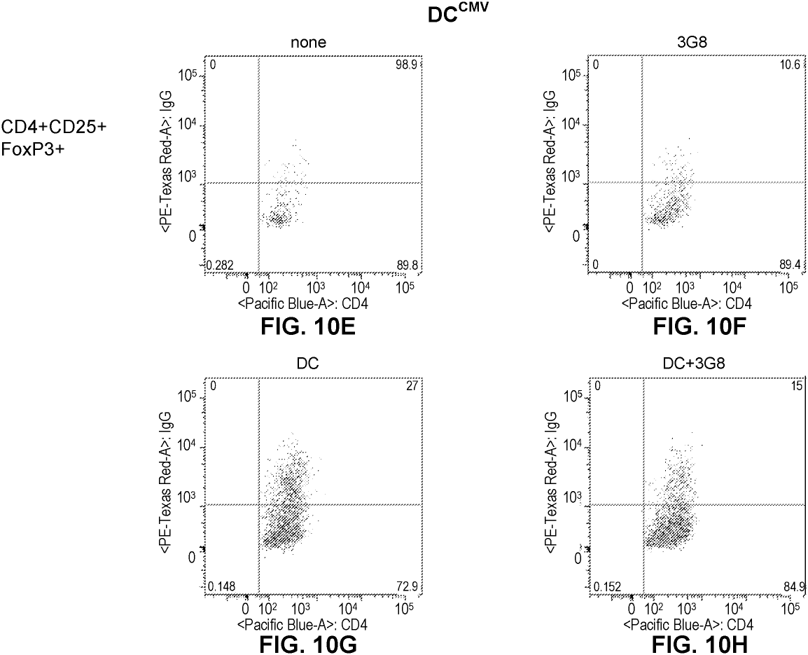

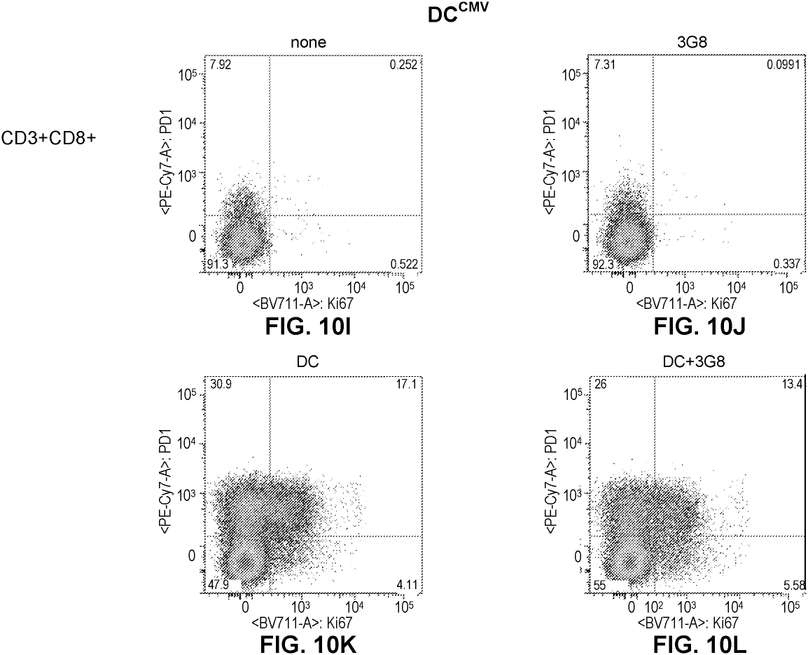

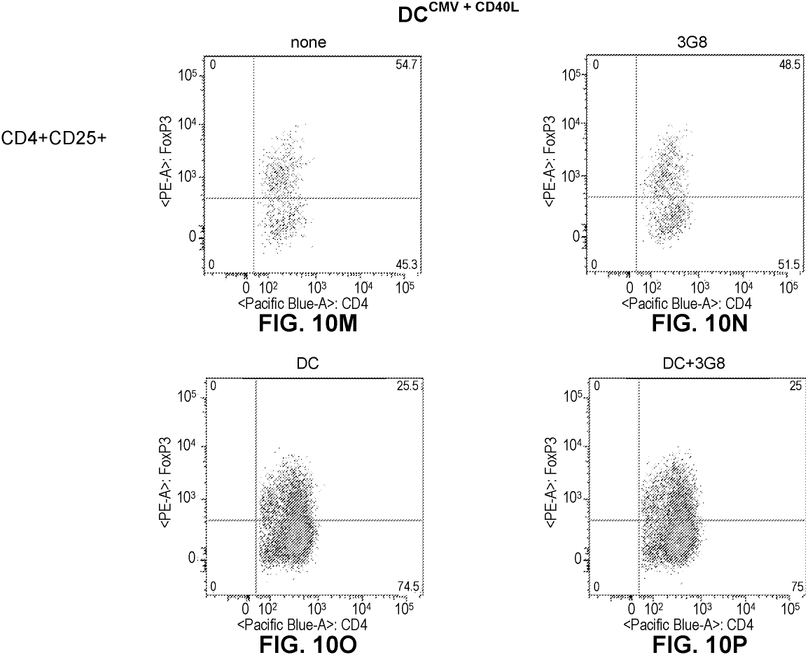

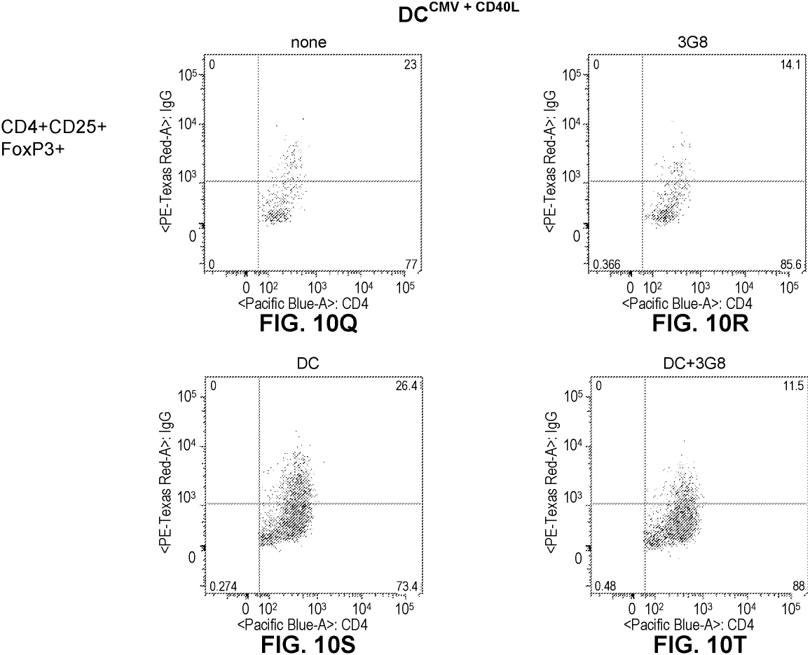

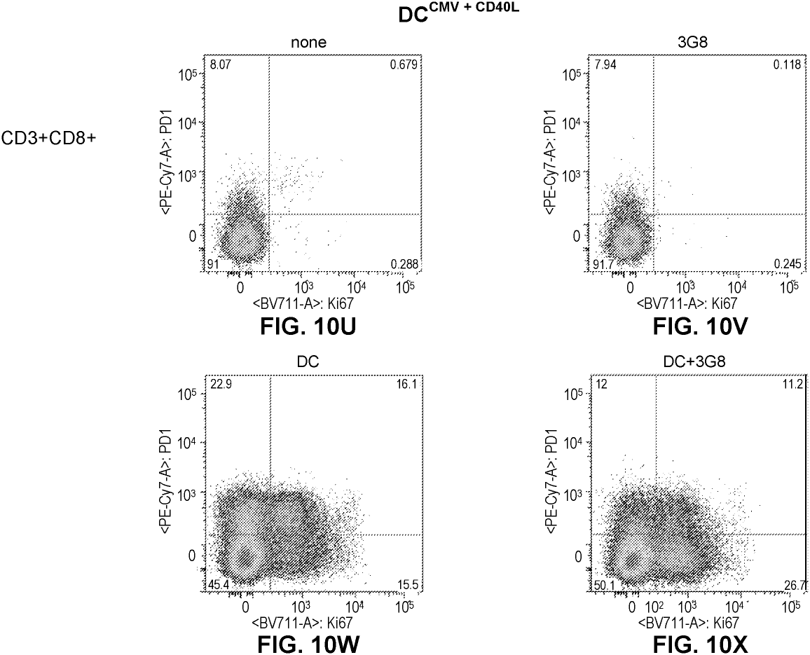

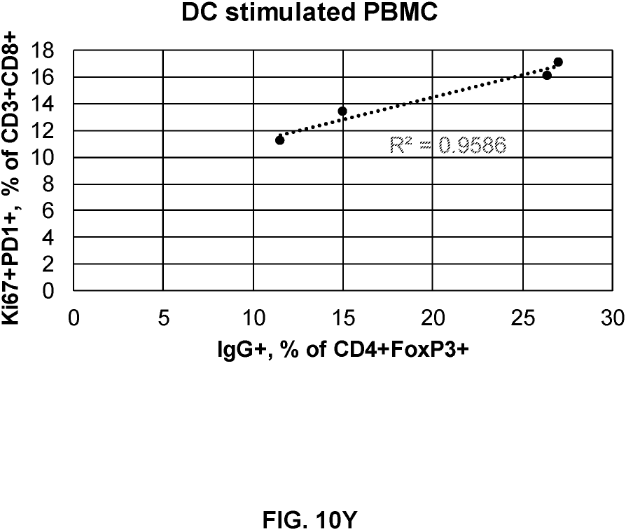

Flow cytometry analysis reveals IgG-immune complex binding and internalization by CD4⁺ T cells during 8 days of DCs stimulation. B cell depleted PBMCs (FIG. 8C) or 10 µg of the anti-CD16 antibody clones 3G8 (FIG. 8D), B73.1 (FIG. 8D), or CB16 (FIG. 8D) added to culture at day 0 show loss of IgG detection in CD4^{high} expressing T cells relative to the cells without the added anti-CD16 antibody (FIG. 8A) or with added isotype control antibody MPOC-21 (10 µg) (FIG. 8B). FIG. 8G shows the decrease in the percentage of CD3⁺CDd^{hi} cells binding IgG in the presence of MPOC-21, 3G8, B73.1 or CB16, or with B cell depletion of PBMCs from an mRCC patient.

### Anti-CD16 Antibody Down Regulates PD1 Expression in CD8⁺ T Cells Induced by PME-CD40L DCs from an mRCC Patient

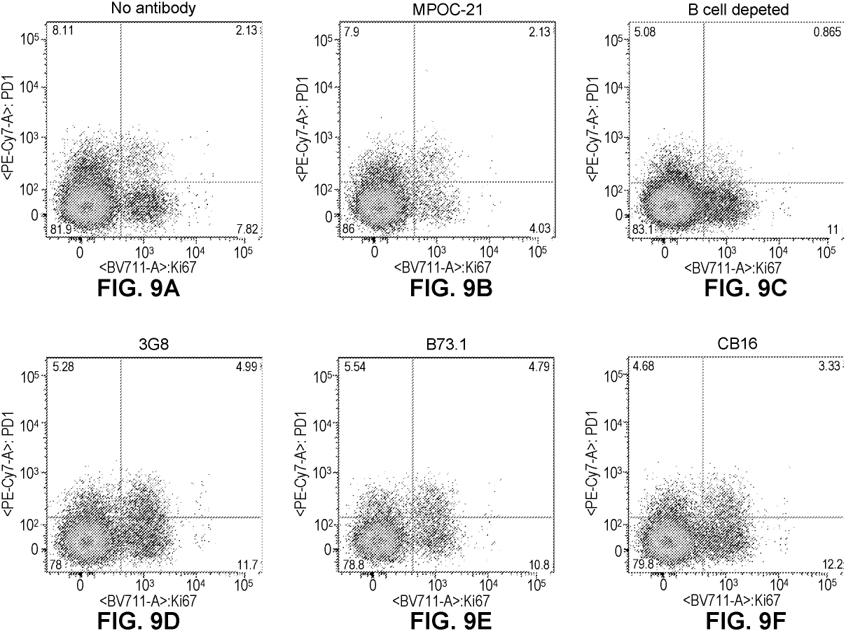

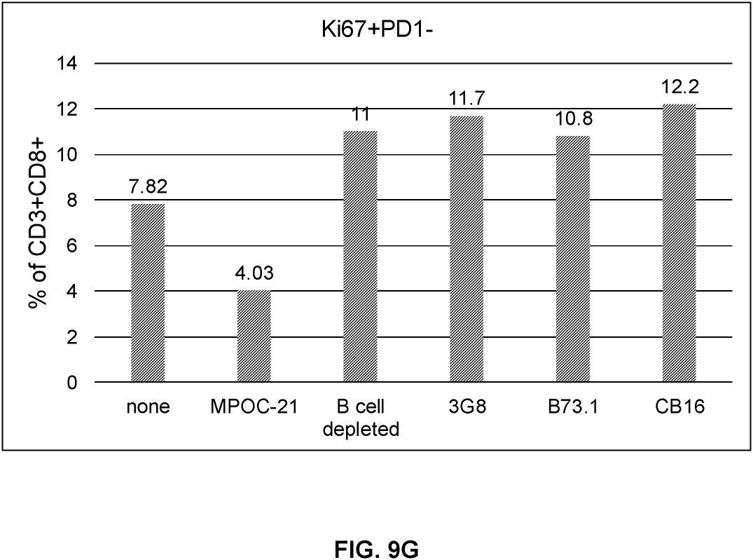

Flow cytometry analysis reveals increased percentages of PD1 negative proliferating (Ki67⁺) CD8⁺T cells (7.82% and 2.13% respectively) after 8 days of DCs stimulation (FIG. 9A). B cell depleted PBMCs (FIG. 9C) or 10 µg of the anti-CD16 antibody clones 3G8 (FIG. 9D), B73.1 (FIG. 9E), or CB16 (FIG. 9F) added to culture at day 0 show increased PD1 negative proliferating CD8⁺ T cells relative to the cells without the added anti-CD16 antibody or with added isotype control antibody MPOC-21 (10 µg) (FIG. 9B). FIG. 9G shows the change in the percentage of PD1 negative proliferating (Ki67⁺) CD8⁺ T cells in the absence of an anti-CD16 antibody, in the presence of MPOC-21, 3G8, B73.1 or CB16, or with B cell depletion of PBMCs from an mRCC patient.

### IgG-Immune Complex Binding and Internalization by CD4⁺T Cells Induces PD1⁺CD8⁺ Proliferating T Cells from Healthy Donors

Mononuclear cells were isolated from a healthy donor using Histopaque-1077 (Sigma-Aldrich), Re-suspended in FBS (Atlanta Biologicals) containing 10% DMSO (Sigma-Aldrich) and stored in liquid nitrogen. Thawed PBMCs were re-suspended in 20 ml of X-Vivo 15 (Lonza). PBMC were re-suspended in X-Vivo 15 containing 5% heat inactivated human AB serum (Valley Biomedical) at a concentration of 1 x 10⁶ cells/ml and incubated for 7 days with or without autologous generated PME-CD40L DCs electroporated with RNA encoding pp65 CMV protein (DC^{CD40L+CMV}) or PME DCs electroporated with RNA encoding pp65 CMV protein only (DC^{CMV}) at 37° C. ± 1° C., 5% CO2 ± 1%, 65-95% relative humidity. Day 7 cultured PBMCs from a healthy donor showed increased FoxP3 expression in CD4⁺CD25⁺ T cells when stimulated with DC^{CMV} at day 0 (FIGS. 10C and 10D). CD4⁺FoxP3⁺T cells showed decreased IgG detection when the anti-CD16 clone 3G8 is added to culture at day 0 with DC^{CMV} (27% (FIG. 10G) to 15% (FIG. 10H)), or DC^{CD40L+CMV} (26.4% (FIG. 10S) to 11.5% (FIG. 10T)). Along with decreased IgG in CD4⁺FoxP3⁺cells, CD3⁺CD8⁺ T cells showed decreased percentages of PD1 positive Ki67⁺ proliferating cells when stimulated with DC^{CMV} (17.1% (FIG. 10K) to 13.4% (FIG. 10L)) or stimulated with DC^{CD40L+CMV} (16.1% (FIG. 10W) to 11.2% (FIG. 10X)). DC^{CD40L+CMV} stimulated PBMCs show greater percentage of proliferating CD8⁺T cells negative for PD1 expression (15.5% 10 W) compared to DC^{CMV} stimulated PBMCs (4.1% (FIG. 10K). The addition of 3G8 antibody further increased the percentage of PD1 negative Ki67+ proliferating CD3+CD8⁺ T cells when added to culture at day 0, 5.6% for DC^{CMV} (FIG. 10L) versus 26.7% for DC^{CD40L+CMV} (FIG. 10X). Correlation analysis for DCs stimulated PBMCs shows a positive correlation for IgG detection in CD4⁺FoxP3⁺ and PD1 expression on proliferating CD3⁺CD8⁺ T cells (FIG. 10Y). Therefore, there is a direct correlation between CD4⁺ cells that bind IgG complexes and CD8⁺PD1⁺ T cells. When IgG complex binding is blocked with the anti-CD16 antibody, there is an increase in the percentage of PD1 negative proliferating CD8⁺T cells.

### Inhibition of IgG-Immune Complex Binding and Internalization by CD4⁺ T Cells Decreases Antigen Specific CD3⁺CD8⁺ CTL PD1 Expression

Mononuclear cells were isolated from a healthy donor using Histopaque-1077 (Sigma-Aldrich), re-suspended in FBS (Atlanta Biologicals) containing 10% DMSO (Sigma-Aldrich) and stored in liquid nitrogen. Thawed PBMCs were re-suspended in 20 ml of X-Vivo 15 (Lonza). PBMCs were re-suspended in X-Vivo 15 containing 5% heat inactivated human AB serum (Valley Biomedical) at a concentration of 1 x 10⁶ cells/ml and incubated for 7 days with or without autologous generated PME-CD40L DCs electroporated with RNA encoding pp65 CMV protein (DC^{CD40L+CMV}) at 37° C. ± 1° C., 5% CO2 ± 1%, 65-95% relative humidity. Some PBMCs were re-stimulated with DC^{CD40L+CMV} on day 6. CD3⁺CD4⁺CD25⁺CD45RA T cells show decreased IgG detection when anti-CD16 clone 3G8 is added to culture at day 0 (FIGS. 11B and 11D). The IgG negative Ki67⁺cells increase from 59.2% (FIG. 11A) to 69.1% (FIG. 11B) for PBMCs with 3G8 not re-stimulated, and 50.1% (FIG. 11C) to 79.6% (FIG. 11D) when re-stimulated with DCs on day 6 and 3G8 on day 0. CMV dextramer positive CD8 T cells show decreased PD1 mean fluorescence intensity when stimulated with DC^{CD40L+CMV} in the presence of 3G8 antibody cultures stimulated for 6 days (727 (FIG. 11I) versus 370 (FIG. 11J)), and 1276 (FIG. 11K) versus 796 (FIG. 11L) for PBMC re-stimulated with DCs on day 6 for an additional day.

### Blocking IGAgs Binding Enhances IFN-γ Secretion by Antigen Specific Memory T Cells

A healthy donor PBMCs were thawed and re-suspended in 20 ml of X-Vivo 15 (Lonza). PBMC were re-suspended in X-Vivo 15 containing 5% heat inactivated human AB serum (Valley Biomedical) at a concentration of 1 x 106 cells/ml and incubated for 8 days with or without autologous generated PME-CD40L DCs electroporated with RNA encoding pp65 CMV protein (DC^{CD40L+CMV}) at 37° C. ± 1° C., 5% CO2 ± 1%, 65-95% relative humidity. The functional consequence of blocking the binding of IGAg complexes to CD16 were tested. Cell generated supernatant was analyzed for the presence of interferon gamma (IFN-γ) using the Cytometric Bead Array (CBA) Flex Set Kit (BD Biosciences) according to the manufacturer’s instructions. 50 µl of supernatant were incubated at room temperature with Capture Beads specific for IFN-γ. After 1 hour, PE detection reagent was added to each tube and allowed to incubate for an additional 2 hours. Samples were washed by adding 1.0 ml of Wash Buffer and centrifuged for 5 minutes at 200 x g. Supernatant was removed from each tube and replaced with 300 µl of Wash Buffer. Samples were acquired using a LSRII flow cytometer and analyzed using FCAP Array software (BD Bioscience). In the presence of the anti-CD 16 antibody 3G8, IFN-γ secretion was enhanced in DC^{CD40L+CMV} stimulated cultures (FIG. 12).

### Blocking IgG Complex Binding Enhances Grb by Antigen Specific Memory T Cells

By blocking IgG complex binding to CD4⁺ T cells (FIG. 13C), there is a subsequent increase in the percentage of CMV specific CTLs from 0.818% (FIG. 13A) to 2.05% (FIG. 13B) in DC^{CD40L+CMV} stimulated cultures. Furthermore, the ability of the CMV specific CTLs to express Grb, a molecule responsible for developing cytolytic activity was increased in the presence of the blocking antibody (FIG. 13D; open histogram).

### Increased Activated NK Cells and Memory T Cells with Decreased IgG Binding Regulatory Cells

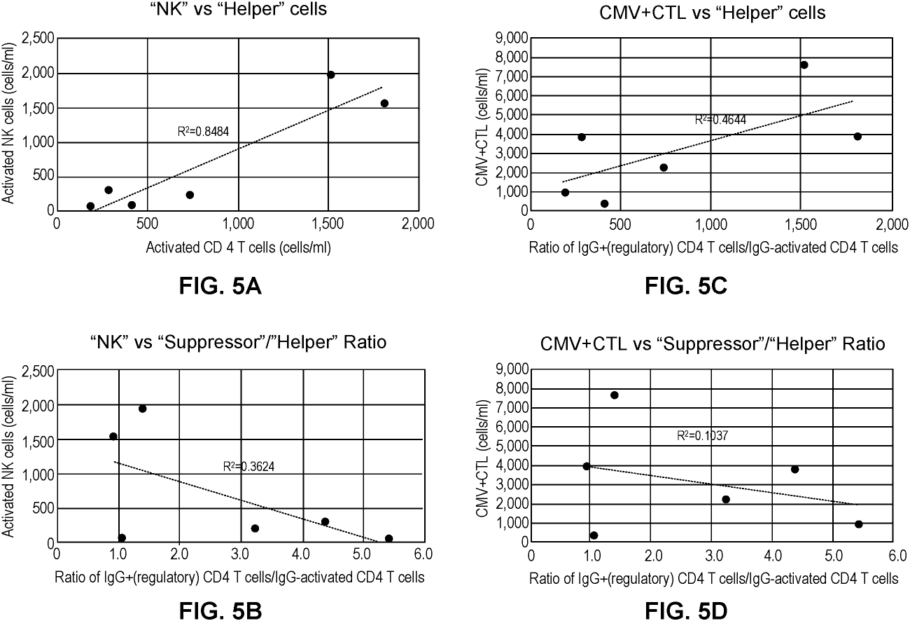

Data generated from CTL cultures stimulated with CMV antigen encoding DCs (as discussed above) was plotted to show the relationship between activated NK cells and activated CD4⁺ T cells (FIG. 5A), activated NK cells and IgG binding regulatory CD4⁺ cells (FIG. 5B), CMV⁺ (specific) CTLs and activated CD4 T cells (FIG. 5C), and CMV⁺ CTLs and IgG binding regulatory CD4 T cells (FIG. 5D).

### Example 6

### Kaplan-Meier Analysis of Overall Survival in the Phase 3 Trial of Autologous Dendritic Cell Immunotherapy Plus Standard Treatment of Advanced Renal Cell Carcinoma (ADAPT)

The full results of the ADAPT clinical trial evaluating an autologous DC therapy in combination with a TKI as 1^{st} line therapy in patients with mRCC were described in Figlin RA. et al., Clin Cancer Res., 26(10):2327-2336 (2020). During a retrospective analysis of the clinical data from the ADAPT trial a cohort of 91 patients was identified who received the mTOR inhibitor everolimus as 2^{nd} line therapy after administration of the DC therapy or after 1^{st} line of SOC therapy. mRCC patients receiving everolimus in combination with a DC therapy exhibited clinical benefit (FIG. 14A). The everolimus treated population (n=91) included subjects who received doses of (a DC therapy) (n=60) or at least 1 or more doses of sunitinib (n=31). The median overall survival (OS) in the patients treated in combination with a DC therapy and everolimus was 23.0 months compared to 13.6 months in the group of patients treated with everolimus alone, with a Hazard Ratio of 0.76 (95% CI; 0.45-1.31). Further retrospective analysis of data from the ADAPT clinical trial looked at those patients (n=49) who received everolimus in the second line after progression showed first, if patients received everolimus during the treatment phase with the DC therapy (FIG. 14B) (n=38) or with everolimus alone (n=11). The median OS in the Combination Treatment Arm was 19.3 months and 17.8 months in the everolimus alone treatment Arm with a Hazard Ratio of 0.89 (95% CI; 0.42-2.10).

The greatest clinical benefit was seen in patients enrolled on the combination arm who received everolimus during the follow up phase of the trial (n=42) (FIG. 14C),. The median OS in the combination group of mRCC patients receiving DC therapy in combination with everolimus (n=22) was 25.8 months compared to 13.4 months in the group treated with everolimus in follow up after SOC therapy (n=20). The Hazard Ratio was 0.51 (95% CI; 0.23-1.11). These results show that everolimus administered in 2^{nd} line therapy provides improved clinical benefit when combined with DC therapy. Moreover, this data suggest that the immune response needs to be activated prior to everolimus addition. Therefore, DC therapy administered prior to everolimus treatment induces an active memory T cell response that is further augmented by everolimus.

Moreover, when patients were compared within the arms, there was good separation of the survival curves between patients who received everolimus during the follow up phase of the trial versus treatment in the combination arm with an OS of 25.7 months compared to 19.3 months (HR 0.59 95%CI; 0.30, 1.19) (FIG. 14D). However, when a similar analysis was performed on patients enrolled in the SOC arm who received everolimus in either follow up or treatment, the survival curves favored administration of everolimus during treatment versus during follow up, with an OS of 17.7 months and 13.4 months respectively (HR 1.04 95%CI; 0.43, 2.48) (FIG. 14E).

### Example 7

### Plasma Collected from mRCC Patients is Immune Suppressive

PBMCs collected from healthy donors were stimulated in vitro with autologous PME-CD40L-CMV DCs encoding the pp65 CMV protein for 7 days in the presence of 10% HD plasma or 10% mRCC patient plasma added on day 0. T cell proliferation was measured by detection of Ki67 positive cells. DC stimulation results in proliferation of both CD4⁺ T cells and CD4⁻(CD8⁺) T cells (FIG. 15A) and the addition of plasma from a healthy donor (FIG. 15B) did not inhibit proliferation in contrast to plasma from a mRCC patient which completely blocked both CD4 and CD8 T cell proliferation (FIG. 15C). Plasma collected from a second healthy donor and tested in a second experiment confirmed the observation that the immune suppressive nature of plasma is specific to plasma collected from mRCC patients and not healthy donors (FIG. 15D).

To understand the scope of immune suppression mediated by mRCC patient plasma a series of experiments were set up utilizing a two-way MLR with HLA mismatched healthy donors to stimulate T cells in the presence of mRCC patient plasma.

The frequency of Ki-67⁺ expression of CD4⁺ and CD4⁻ T cells was determined for no plasma addition (left panel) and plasma treated (right panel) samples in a two-way MLR after 7 days of culture (FIG. 16A). The addition of mRCC patient plasma blocked both CD4⁺ and CD8⁺ T cell proliferation, reducing the frequency of proliferating CD4 T cells from 12% to 0.26% (FIG. 16A upper right quadrants) and proliferating CD8 T cells from 5.36% to 0.58% for CD8 T cells (FIG. 16A lower right quadrants).

Moreover, OKT3 antibody was added to PMBC cultures on day 0 and proliferation of CD4⁺ and CD4⁻ T cells was determined by detection of Ki-67 by flow cytometry for no plasma addition (left panel) and plasma treated (right panel) samples after 7 days of culture (FIG. 16B). When PBMC cultures where stimulated with the anti-CD3 antibody OKT3, which bypasses MHC restriction and directly signals through the TCR complex, the addition of mRCC patient plasma did not suppress CD4 T cell (FIG. 16B upper right quadrants) nor CD8 T cell proliferation (FIG. 16B lower right quadrants). These data show that the ability of mRCC patient plasma to inhibit T cell proliferation was dependent on recognition of MHC molecules on the APCs in the culture. The suppression profile in the MLR is shown for replicate samples (FIG. 16C).

Moreover, stimulation of MLR cultures with mRCC patient plasma reduced the expression of both CD25 (FIG. 16D) and CD28 (FIG. 16E) receptors on CD4 and CD8 T cells. In contrast, the addition of mRCC patient plasma had little impact on the expression of CD25 when T cells were stimulated with OKT3 antibody (FIG. 16D), but did impact the expression of CD28 similar to the effect seen in the MLR (FIG. 16E).

Given the observation that patients enrolled in the ADAPT study who received everolimus as 2^{nd} line therapy after receiving DC therapy had better clinical outcomes, the concept that everolimus may reverse the immune suppressive activity of mRCC patient plasma and augment T cell responses was investigated. HLA-mismatched healthy donor PBMCs were mixed and cultured for 7 days in the presence of 2% mRCC patient plasma and with or without 20 ng of everolimus added on day 0, and the frequency of CD28⁺ memory T cells was determined. Everolimus partially restored the frequency of proliferating CD28⁺ CD4 T cells reduced in the presence of mRCC patient plasma (FIG. 17A middle and right panels). However, this restoration was not statistically significant (p<0.087) when the suppressive activity of plasma samples collected from six individual mRCC patients was tested (FIG. 17C). In contrast, an increase in the frequency of proliferating CD28⁻CD4 T cells was not detected with plasma addition, which could be reversed with the addition of everolimus (FIG. 17D). A similar pattern was detected for CD8 T cells (a decrease in the frequency of proliferating CD28⁺ CD8 T cells (FIG. 17E) and increase in the frequency of proliferating CD28⁻CD8 T cells (FIG. 17F)) in the presence of plasma, which the addition of everolimus could reverse. These results reached statistical significance for the cohort of six mRCC patient plasma samples tested. To expand the understating whereby everolimus can reduce the immune suppressive impact of mRCC, patient plasma PBMCs were stimulated with autologous DCs presenting the pp65 CMV antigen. Healthy donor PBMCs and autologous DCs were mixed and cultured for 6 days in the presence of 2% mRCC patient plasma and with or without 20 ng of everolimus added on day 0. A small but reproducible frequency of proliferating CD28⁺ CD4 T cells was induced, but very few CD28⁻CD4 T cells (FIG. 18A left panel), which is unlike the CD4 T cell response seen in the MLR. However, the frequency of proliferating CD28⁺ CD4 T cells was suppressed in the presence of mRCC patient plasma (FIG. 18A middle panel) and partially restored when everolimus was added to the co-cultures (FIG. 18A right panel). Similar to results from the MLR, the change in the proliferative response in the presence of everolimus did not reach statistical significance in the cohort of six plasma samples tested (p<0.283) (FIG. 18C). A low frequency of proliferating CD28⁻ CD4 T cells was detected, and everolimus has little impact on the frequency of these cells (FIG. 18D). However, everolimus partially reversed plasma mediated suppression of proliferating CD28⁺ CD8 T cells (FIG. 18B middle and right panels) and this effect was statistically significant for the cohort of six plasma samples tested (p< 0.0002) (FIG. 18E). In contrast to data seen in the MLR, everolimus had minimal ability to decrease the frequency of proliferating CD28⁻CD8 T cells (p<0.92) (FIG. 18F).

### Example 8

### Everolimus Helps to Sustain the Frequency of CD25⁺CD28⁺ Memory T Cells

While everolimus is an approved therapy for the treatment of renal cell carcinoma, along with other mTOR inhibitors, it has traditionally been used as an immune suppressant to prolong organ transplant survival, particularly kidney graft survival. Therefore, it was of interest to understand how an immune suppressant would provide clinical benefit to patients receiving an autologous DC therapy designed to induce memory T cell responses. See e.g., DeBenedette MA et al., J Immunother., 34(1):45-57 (2011); Calderhead DM. et al., J Immunother., 31(8):731-741 (2008).

Given the observation that plasma suppression selectively impacts the frequency of CD28⁺ T cells and everolimus mediates a reduced suppression of this memory phenotype, the CD28⁺ T cell subset was further characterized. HLA-mismatched healthy donor PBMCs were mixed and cultured for 7 days in the presence of 2% mRCC patient plasma and with or without 20 ng of everolimus added on day 0. Increased frequencies of double positive CD28 and CD25 CD4 and CD8 T cells were detected in the MLR cultures post-stimulation (FIGS. 19A and 19B left panels) suggesting these are early memory T cells. Both the expression of CD25 and CD28 receptors on T cells are critical for full differentiation of memory T cells. Plasma treatment decreased the frequency of both CD25⁺CD28⁺ CD4 and CD8 T cells (FIGS. 19A and 19B middle panels). Everolimus addition to cultures stimulated in the presence of plasma resulted in partial restoration of the frequencies of CD25⁺CD28⁺ CD4 and CD8 T cells (FIGS. 19A and 19B right panels). The composite results from cohort of cultures treated with six independent plasma samples show that restoration of the CD25⁺CD28⁺ T cell phenotype is statistically significant for both CD4 T cells (FIG. 19C p< 0.045) and CD8 T cells (FIG. 19E p< 0.0061). Moreover, the increase in the frequency of CD25⁺CD28⁻ CD8 T cells in cultures stimulated in the presence of plasma no longer represented the dominant population of CD25⁺CD8 T cells when everolimus was added (FIG. 19D p< 0.001).

Furthermore, the impact of everolimus on the suppressive effect of mRCC patient plasma when PBMCs were stimulated with autologous DCs was examined. Similar to data presented from the MLR experiments, the frequency of CD25⁺CD28⁺ CD4 and CD8 T cell populations was decreased in the presence of mRCC patient plasma (FIGS. 20A and 20B left and middle panels) and could be partially restored with the addition of everolimus (FIGS. 20A and 20B right panels). However, the restoration of CD25⁺CD28⁺ CD4 T cells did not reach statistical significance in the cohort of six mRCC plasma samples tested (FIG. 20C). In contrast everolimus was able to partially restore the frequency of CD25⁺CD28- (FIG. 20D) and CD25⁺CD28⁺ (FIG. 20E) CD8 T cells stimulated in the presence of mRCC patient plasma reaching statistical significance for the cohort of six patient plasma samples tested.

To understand the ability of everolimus to restore the frequency of CD25⁺CD28⁺ T cells, the expression of both CD25 and CD28 on CD8 T cells stimulated in the MLR or with autologous DCs in the presence of mRCC patient plasma was measured by the geometric mean fluorescence intensity (MFI). T cell responses were measured after stimulating HLA-mismatched healthy donor PBMCs in a MLR for 7 days (FIGS. 21A and 21B) or with DCs and cultured for 6 days in the presence of 2% mRCC patient plasma and with or without 20 ng of everolimus added on day 0 (FIGS. 21C and 21D). mRCC patient plasma decreased the expression of both CD25 and CD28 on the cell surface of CD8 T cells stimulated in the MLR or with autologous DCs (FIG. 21). Everolimus addition resulted in a statistically significant increase in the expression of CD25 and CD28 on CD8 T cells stimulated in the MLR (FIGS. 21A and 21B) and stimulated with autologous DCs (FIGS. 21C and 21D). Whereby there was a statistically significant increase in the expression of CD25 (FIGS. 21A and 21C) and CD28 (FIGS. 21B and 21D) on the cell surface of CD8 T cells.

### Example 9

### Everolimus Inhibits B Cell Secretion of TGF-P

One potential immune suppressive component of plasma collected from mRCC patients is the high concentrations of TGF-β. The tumor can create an immune suppressive microenvironment through the presence of TGF-β leading to dysregulated CD4 and CD8 T cell function. In a cohort of eight randomly selected ADAPT mRCC patient plasma samples tested high concentrations of TGF-β (ranging from 12 ng/mL to 143 ng/mL) was detected as compared to plasma collected from two individual healthy donors (1.9 ng/mL and 3.4 ng/mL) (FIG. 22A). The concentration of TGF-β measured in the plasma of mRCC patients inversely correlated with the frequency of Ki-67⁺ CD4 T cells stimulated in the MLR cultures (FIG. 22B; r² values 0.51).

One potential source of TGF-β are regulatory B cells, and the ability of B cells to produce TGF-β can be regulated through the mTOR signaling pathway. Moreover, activated CD38⁺B cells present in the peripheral blood of mRCC patients produce latent TGF-β. It has been reported that CD38⁺ B cells are a source of regulatory B cells that suppress autoimmune inflammatory responses and CD4 T cells differentiation via IL-10, but not by TGF-β. See e.g., Bankó Z, J. Immunol., 198(4): 1512-1520 (2017); Blair PA. et al., Immunity, 32(1): 129-140 (2010). However, the frequency of CD38⁺ B cells and TGF-β were positively correlated in patients with periodontal disease. See e.g., Hetta HF. et al., Vaccines, 8(2) (2020).The discrepancies in the reported data may reflect the different inflammatory environment, giving rise to B cells with different cytokine profiles. See e.g., Rosser EC. et al., Immunity, 42(4):607-612 (2015).

Thus, the impact of everolimus on B cell TGF-β production was investigated. PBMCs from mRCC patients were stimulated (“stim”) or left unstimulated (“control”) with or without everolimus addition as described in Example 1 to induce TGF-β secretion. TGF-β concentrations in the supernatant were measured after 6 days by ELISA. When PBMCs from mRCC patients where stimulated to activate B cells in the presence of everolimus, there was a decrease in concentration of TGF-β secreted by stimulated B cells (FIG. 23A).

The frequency of CD38⁺ B cells was determined by flow cytometry by gating on the viable CD19⁺ B cells in unstimulated cultures (“control”) or in stimulated cultures (“stim”) with or without everolimus addition. Stimulation resulted in the detection of CD38⁺ B cells ranging from 5.58% to 22.1% of the CD19⁺ B cells, and when treated with everolimus there was a subsequent reduction in the frequency of CD38⁺B cells ranging from 3.07% to 6.35% (FIG. 23B). Stimulated CD19⁺ B cells were capable of producing TGF-β, by intracellular detection of the latent form of TGF-β that is complexed with the latency-associated peptide (LAP) (FIG. 23C top right panel).

Treatment with everolimus resulted in a decrease in the frequency of CD19⁺ LAP⁺ B cells (FIG. 23C bottom right panel). Furthermore, sub-gating of the LAP⁺ B cells revealed that everolimus reduced the frequency of LAP⁺/Ig⁺ B cells from 61.5% to 6.14% (FIG. 23D upper left quadrants) and reduced frequencies of LAP⁺/Ig⁺ CD38⁺ B cells from 3.89% to 0.11% (FIG. 23D upper right quadrants) and LAP⁺CD38⁺Ig- B cells from 4.04% to 2.63% (FIG. 23D lower right quadrants). Therefore, everolimus can modulate the ability of B cells to differentiate into CD38⁺ TGF-β⁺ B cells.

The ability to detect TGF-β in activated CD38⁺B cells from cancer patients may reflect the regulatory state of B cells in cancer patients. TGF-β does not exist in an active form, and must be cleaved from the latent protein for activation. This suggests a link between TGF-β in the plasma and the B cells as a mediator to deliver the TGF-β to T cells.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Other aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All publications, patents, and patent applications disclosed herein are incorporated by reference to the same extent as if each individual publication, patent or patent application was specifically and individually indicated to be incorporated by reference.

What is claimed is:

1. A method of treating a tumor, comprising the sequential steps of:
(a) administering a dose regimen of an immunotherapy comprising dendritic cells loaded with RNA encoding a tumor antigen to a patient having a tumor; and
(b) after administration of at least one dose of the immunotherapy of step (a), administering a dose regimen of a pharmaceutical which can cause one or more of the following: (i) decrease circulating IgG levels, (ii) block IgG-mediated activation of CD16⁺ T cells, (iii) decrease the concentration and/or function of B cells, (iv) reduce the frequency of CD38⁺ TGF-β⁺ B cells, (v) decrease B cell secretion of TGF-β, and (vi) sustain the frequency of CD25⁺CD28⁺ CD4 and/or CD8 T cells.

2. The method of claim 1, wherein administration of the pharmaceutical occurs after tumor progression.

3. The method of claim 2, wherein the pharmaceutical is an mTOR inhibitor.

4. The method of claim 3, wherein a first dose of the mTOR inhibitor is administered after progression of the tumor.

5. The method of claim 3, wherein the mTOR inhibitor is rapamycin or a rapamycin analog.

6. The method of claim 5, wherein the rapamycin analog is selected from the group consisting of everolimus, temsirolimus, sirolimus, and ridaforolimus.

7. The method of claim 6, wherein the rapamycin analog is everolimus.

8. The method of clam 7, wherein everolimus is administered about once per day.

9. The method of claim 8, wherein about 10 mg of everolimus is administered once per day.

10. The method of claim 6, wherein the rapamycin analog is temsirolimus.

11. The method of claim 6, wherein temsirolimus is administered once per week.

12. The method of claim 11, wherein about 25 mg of temsirolimus is administered once per week.

13. The method of claim 1, wherein the pharmaceutical decreases the function of B cells and is selected from the group consisting of: natalizumab, teriflunomide, and ofatumumab.

14. The method of claim 1, wherein the pharmaceutical decreases the concentration of B cells and is selected from the group consisting of: prednisone, cyclophosphamide, methotrexate, mycophenolate mofetil, azathioprine, trimetrexate, cortisol, prednisolone, methylprednisolone, dexamethasone, metamethasone, triamcinolone, denosumab, triamcinolone acetonide, atacicept, ocrelizumab, obinutuzumab, bevacizumab, and inotuzumab ozogamicin.

15. The method of claim 1, wherein the pharmaceutical decreases circulating levels of IgG and is selected from the group consisting of carbamazepine, sodium valproate, phenobarbital, phenytoin, lenalidomide, cloroquine, quinine, amodiaquine, pyrimethamine, proguanil, sulfonamides, mefloquine, atovaquone, primaquine, artemisinin, halofantrine, doxycycline, clindamycin, captopril, cortisol, prednisone, prednisolone, methylprednysolone, dexamethasone, metamethasone, trimcinolone, fludrocortisone acetate, deoxycorticostetone acetate, fenclofenac, gold salts, penicillamine, and sulfasalazine.

16. The method of claim 1, wherein the pharmaceutical blocks IgG-mediated activation of CD16⁺ T cells.

17. The method of claim 16, wherein the pharmaceutical is an anti-CD16 antibody or an antibody that cross-competes with the anti-CD16 antibody for binding to the same epitope or an antibody that binds to the same epitope as the anti-CD16 antibody.

18. The method of claim 17, wherein the pharmaceutical is the 3G8 antibody, the B73.1 antibody or the CB16 antibody, or an antibody that cross-competes with the 3G8 antibody, the B73.1 antibody or the CB16 antibody for binding to the same epitope or an antibody that binds to the same epitope as the 3G8 antibody, the B73.1 antibody, or the CB16 antibody.

19. The method of claim 1, wherein the immunotherapy is CMN-001.

20. The method of claim 19, wherein CMN-001 is administered about once every three weeks.

21. The method of claim 1, where the regimen of immunotherapy continues after the initiation of the dose regimen of the pharmaceutical.

22. The method of claim 1, wherein the tumor is renal cell cancer.

23. The method of claim 22, wherein the tumor is a metastatic renal cell cancer.

24. The method of claim 22, wherein the tumor is the clear cell type.

25. The method of claim 23, wherein the patient is a poor risk human patient.

26. The method of claim 25, wherein the poor risk patient exhibits three or more of the following risk factors:
(i) time from diagnosis to the initiation of systemic therapeutic treatment of less than one year,
(ii) low levels of hemoglobin,
(iii) elevated corrected calcium levels,
(iv) diminished patient performance status or physical functioning,
(v) elevated levels of neutrophils, and
(vi) elevated platelet count.

27. The method of claim 1, wherein the tumor is selected from the group consisting of: breast cancer, pancreatic cancer, astrocytoma, glioblastoma multiforme, melanoma, lymphoma, and Waldenstrom macroglobulinaemia.

28. The method of claim 1, wherein the tumor antigen is autologous to the patient.

29. A method of decreasing circulating IgG levels, blocking IgG-mediated activation of CD16⁺ T cells, and/or decreasing the concentration and/or function of B cells in a patient having a tumor, comprising the sequential steps of:
(a) administering a dose regimen of an immunotherapy comprising dendritic cells loaded with RNA encoding a tumor antigen to the patient; and
(b) after administration of at least one dose of the immunotherapy of step (a), administering a dose regimen of a pharmaceutical which can cause one or more of the following: (i) decrease circulating IgG levels, (ii) block IgG-mediated activation of CD16⁺ T cells, (iii) decrease the concentration and/or function of B cells, (iv) reduce the frequency of CD38⁺ TGF-β⁺ B cells, (v) decrease B cell secretion of TGF-β, and (vi) sustain the frequency of CD25⁺CD28⁺ CD4 and/or CD8 T cells.

30. A method of modulating Programmed Cell Death Protein 1 (PD1) expression on CD8⁺ T cells in a patient having a tumor, comprising the sequential steps of:
(a) administering a dose regimen of an immunotherapy comprising dendritic cells loaded with RNA encoding a tumor antigen to the patient; and
(b) after administration of at least one dose of the immunotherapy of step (a), administering a dose regimen of a pharmaceutical which can cause one or more of the following: (i) decrease circulating IgG levels, (ii) block IgG-mediated activation of CD16⁺ T cells, (iii) decrease the concentration and/or function of B cells, (iv) reduce the frequency of CD38⁺ TGF-β⁺ B cells, (v) decrease B cell secretion of TGF-β, and (vi) sustain the frequency of CD25⁺CD28⁺ CD4 and/or CD8 T cells.
